(19) United States
(12) Patent Application Publication
(10) Pub. No.: US 2020/0185615 A1
(43) Pub. Date: Jun. 11, 2020

(54) ORGANIC CHEMICAL COMPOUND, ORGANIC MIXTURE, AND ORGANIC ELECTRONIC COMPONENT

(71) Applicant: GUANGZHOU CHINARAY OPTOELECTRONIC MATERIALS LTD., Guangzhou, Guangdong (CN)

(72) Inventors: Ruifeng HE, Guangzhou, Guangdong (CN); Yini LI, Guangzhou, Guangdong (CN); Junyou PAN, Guangzhou, Guangdong (CN)

(21) Appl. No.: 16/463,632

(22) PCT Filed: Nov. 23, 2017
(86) PCT No.: PCT/CN2017/112714

§ 371 Date: May 23, 2019

(30) Foreign Application Priority Data
Nov. 23, 2016 (CN) 201611047051.5

(51) Int. Cl. H01L 51/00; C07D 401/10; C09K 11/06; C07D 405/10; C07D 213/16

(52) U.S. Cl. CPC H01L 51/0067; C07D 401/10; C09K 11/06; H01L 51/0072; H01L 51/5072; C07D 405/10; H01L 51/0073; C07D 213/16; H01L 51/0056

(57) ABSTRACT

Provided are an organic chemical compound, organic mixture, and organic electronic component; the structure of said organic chemical compound is as shown in general formula (1); the definition of the substituent group in the general formula (1) is the same as in the description.

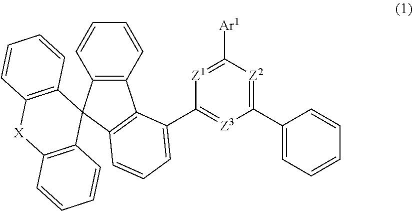

### CROSS-REFERENCE TO RELATED APPLICATIONS

The present application is a national stage for International Application No. PCT/CN2017/112714, filed on Nov. 23, 2017, which claims priority to Chinese Application No. 201611047051.5, filed on Nov. 23, 2016, both of which are incorporated herein by reference in their entireties.

### TECHNICAL FIELD

The present disclosure relates to the technical field of organic opto-electronic materials, particularly to an organic compound, an organic mixture, and an organic electronic device.

### BACKGROUND

Organic semiconductor materials have the characteristics of structural diversity, relatively low manufacturing cost, excellent opto-electronic property, and the like. Therefore, they have great potential for application in opto-electronic devices e.g. organic light-emitting diodes (OLEDs), such as flat panel displays and lighting.

In order to improve the luminescence properties of organic light-emitting diodes and promote the large-scale industrialization process of organic light-emitting diodes, various organic opto-electronic material systems have been widely developed. However, the properties of OLEDs, especially the lifetime of OLEDs, are not high enough.

In view of the molecule, the close packing of organic molecules easily leads to the formation of non-radiative transitions and fluorescence quenching of excitons, structurally, electron-accepting groups, e.g. nitrogen-containing heteroaromatic rings, have relatively good planarity and relatively poor structural stability which greatly affects the processability of opto-electronic materials and the properties and lifetime of opto-electronic devices. Therefore, proper spatial modification and protection of the electron-accepting groups of organic opto-electronic molecules will be beneficial to improve the stability and opto-electronic property of such molecules. Currently, there is still not much research on related technologies. The patent CN104541576A discloses a class of derivatives of triazine or pyrimidine, but the performance and lifetime of the devices obtained remains to be continuously improved.

### SUMMARY

According to various embodiments of the present disclosure, an organic compound, an organic mixture, and an organic electronic device are provided to address one or more of the problems involved in the background.

An organic compound for an organic electronic device has a general formula (1) as following:

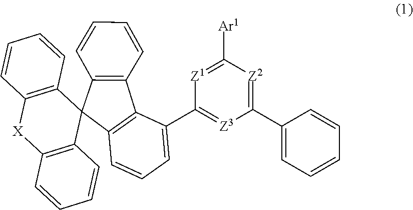

wherein

Z¹, Z², Z³ are independently selected from N or CR¹, and at least one of Z, Z² and Z³ is an N atom;

X is independently selected from the group consisting of a single bond, N(R¹), C(R¹)₂, Si(R¹)₂, O, C═N(R¹), C═C(R)₂, P(R¹), P(═O)R¹, S, S═O, and SO₂;

Ar¹ is selected from an aryl group containing more than 6 ring atoms or a heteroaryl group containing more than 6 ring atoms;

R¹ is selected from the group consisting of H, D, F, CN, carbonyl, sulfonyl, alkoxy, an alkyl group containing 1 to 30 carbon atoms, a cycloalkyl group containing 3 to 30 carbon atoms, and an aryl group containing 5 to 60 ring atoms or a heteroaryl group containing 5 to 60 ring atoms.

A polymer has repeating units at least one of which comprises the foregoing organic compound.

An organic mixture for an organic electronic device comprises at least one organic functional material and the foregoing organic compound, and the organic functional material is selected from the group consisting of a hole injection material, a hole transporting material, a hole blocking material, an electron injection material, an electron transporting material, an electron blocking material, an organic host material and an organic dye or a light-emitting material.

An ink for an organic electronic device comprises an organic solvent, and the foregoing organic compound or the foregoing polymer.

An organic electronic device comprises a functional layer including the foregoing organic compound, or the foregoing organic mixture or the foregoing polymer, or is prepared from the foregoing ink.

Details of one or more embodiments of the present disclosure are set forth in the accompanying drawings and description below. Other features, objects, and advantages of the present disclosure will be apparent from the description, the accompanying drawings and the claims.

### DETAILED DESCRIPTION OF EMBODIMENTS

In order to make the objects, technical solutions and advantages of the present disclosure more clearly, the present disclosure will be further described in detail below with reference to the accompanying drawings and examples. It is understood that the specific examples described herein are merely for illustration of the disclosure and are not intended to limit the disclosure.

In the present disclosure, the formulation, the printing ink and the ink have the same meaning and are interchangeable. The host material and the matrix material have the same meaning and are interchangeable. The metal organic clathrate, the metal organic complex, and organometallic complex have the same meaning and are interchangeable.

In view of the molecule, the close packing of organic molecules easily leads to the formation of non-radiative transitions and fluorescence quenching of excitons. In view of the structure, electron-accepting groups, e.g. nitrogen-containing heteroaromatic rings, have relatively good planarity and relatively poor structural stability which greatly affects the processability of opto-electronic materials and the properties and lifetime of opto-electronic devices. Therefore, proper spatial modification and protection of the electron-accepting groups of organic opto-electronic molecules will be beneficial to improve the stability and opto-electronic property of such molecules.

An organic compound of one embodiment has a general formula (1) as following:

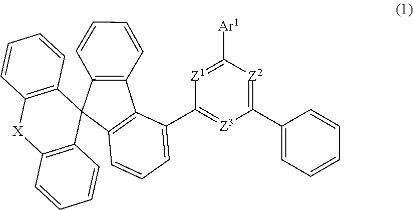

wherein

Z¹, Z², Z³ are independently selected from N or CR¹, and at least one of Z¹, Z² and Z³ is an N atom;

X is independently selected from the group consisting of a single bond, N(R¹), C(R¹)₂, Si(R¹)₂, O, C═N(R¹), C═C(R¹)₂, P(R¹), P(═O)R¹, S, S═O, and SO₂;

Ar¹ is selected from an aryl group containing more than 6 ring atoms or a heteroaryl group containing more than 6 ring atoms;

R¹ is selected from the group consisting of H, D, F, CN, carbonyl, sulfonyl, alkoxy, an alkyl group containing 1 to 30 carbon atoms, a cycloalkyl group containing 3 to 30 carbon atoms, and an aryl group containing 5 to 60 ring atoms or a heteroaryl group containing 5 to 60 ring atoms.

The foregoing organic compound can be used in organic electronic devices, particularly as a light-emitting layer material in organic electronic devices. The nitrogen-containing heteroaromatic ring has relatively good planarity and strong electron-accepting property, which easily leads to the generation of close packing and strong interaction between molecules, so that excitons are prone to non-radiative transition and fluorescence quenching. The foregoing organic compound directly connects the nitrogen-containing heteroaromatic ring to the spirocyclic group having great steric hindrance, effectively preventing close packing between molecules and simultaneously dispersing the electron-accepting effect of the nitrogen-containing heteroaromatic ring, thereby improving the stability of the material and the device and further improving the lifetime of the organic electronic device.

In one embodiment, Ar¹ is selected from an aryl group containing 7 to 60 ring atoms or a heteroaryl group containing 7 to 60 ring atoms. Further, Ar¹ is selected from an aryl group containing 7 to 50 ring atoms or a heteroaryl group containing 7 to 50 ring atoms. Still further, Ar¹ is selected from an aryl group containing 7 to 40 ring atoms or a heteroaryl containing 7 to 40 ring atoms. Even further, Ar¹ is selected from an aryl group containing 7 to 30 ring atoms or a heteroaryl group containing 7 to 30 ring atoms.

An aryl group refers to a hydrocarbyl containing at least one aromatic ring. An aryl group may also be an aromatic ring system which refers to a ring system including monocyclic groups and polycyclic groups. Heteroaryl groups refer to hydrocarbyl groups containing at least one heteroaromatic ring (containing heteroatoms). Among them, the heteroatom is selected from one or more of Si, N, P, O, S, and Ge. Further, the heteroatom is selected from one or more of Si, N, P, O, and S. A heteroaromatic group may also be an aromatic ring system which refers to a ring system including a monocyclic group and a polycyclic group. These polycyclic ring species may have two or more rings where two carbon atoms are shared by two adjacent rings, i.e., a fused ring. At least one ring of these polycyclic ring species is aromatic or heteroaromatic. In the present embodiment, the aromatic or heteroaromatic ring system includes not only a system of an aryl group or a heteroaryl group. The aromatic or heteroaromatic ring systems may also include a plurality of aryl or heteroaryl groups which are interrupted by short non-aromatic units (<10% of non-H atoms, further less than 5% of non-H atoms, such as C, N or O atoms). Therefore, systems such as 9,9′-spirobifluorene, 9,9-diarylfluorene, triarylamine, diaryl ether and the like can be considered as aromatic ring systems.

In one embodiment, the aryl group is selected from the group consisting of benzene, naphthalene, anthracene, phenanthrene, perylene, tetracene, pyrene, benzopyrene, triphenylene, acenaphthene or fluorene, or derivatives thereof.

The heteroaryl group is selected from the group consisting of furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, primidine, quinazoline, quinazolinone, or derivatives thereof.

In another embodiment, at least two of Z¹, Z² and Z³ shown in the general formula (1) are N atoms. Further, all of Z¹, Z² and Z³ are N atoms.

In one embodiment, X shown in the general formula (1) is selected from a single bond, N(R¹), C(R¹)₂, O or S.

In one embodiment, R¹ shown in the general formula (1) is selected from H, D, an alkyl group containing 1 to 20 carbon atoms or a cycloalkyl group containing 3 to 20 carbon atoms, an aryl group containing 5 to 40 ring atoms or a heteroaryl group containing 5 to 40 ring atoms. Further, R¹ is selected from H, D, an alkyl group containing 1 to 10 carbon atoms or a cycloalkyl group containing 3 to 10 carbon atoms, an aryl group containing 5 to 30 ring atoms or a heteroaryl group containing 5 to 30 ring atoms. Still further, R¹ is selected from H, D, an alkyl group containing 1 to 4 carbon atoms or a cycloalkyl group containing 3 to 6 carbon atoms, an aryl group containing 5 to 18 ring atoms or a heteroaryl group containing 5 to 18 ring atoms.

In one embodiment, Ar¹ includes one or more of the following groups:

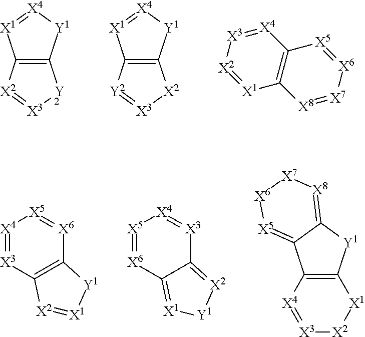

wherein

X¹, X², X³, X⁴, X⁵, X⁶, X⁷ and X⁸ are independently selected from CR² or N;

Y¹ and Y² are independently selected from CR²R³, SiR²R³, NR², C(═O), S or O;

R² and R³ are one or more independently selected from H, D, a linear alkyl group containing 1 to 20 C atoms, a linear alkoxy group containing 1 to 20 C atoms, a linear thioalkoxy group containing 1 to 20 C atoms, a branched alkyl group containing 3 to 20 C atoms or a cyclic alkyl group containing 3 to 20 C atoms, a branched alkoxy group containing 3 to 20 C atoms or a cyclic alkoxy group containing 3 to 20 C atoms, a branched thioalkoxy group containing 3 to 20 C atoms or a cyclic thioalkoxy group containing 3 to 20 C atoms, a branched silyl group containing 3 to 20 C atoms or a cyclic silyl group containing 3 to 20 C atoms, a substituted keto group containing 1 to 20 C atoms, an alkoxycarbonyl group containing 2 to 20 C atoms, an aryloxycarbonyl group containing 7 to 20 C atoms, a cyano group (—CN), a carbamoyl group (—C(═O)NH₂), a haloformyl group (—C(═O)—X, wherein X is selected from halogen atoms), a formyl group (—C(═O)—H), an isocyano group, an isocyanate group, a thiocyanate group, an isothiocyanate group, a hydroxyl group, a nitryl group, a CF₃ group, Cl, Br, F, a crosslinkable group, a substituted or non-substituted aromatic or heteroaromatic ring system containing 5 to 40 ring atoms, and an aryloxy or a heteroaryloxy group containing 5 to 40 ring atoms, wherein at least one of R² and R³ forms a monocyclic or polycyclic aliphatic or aromatic with a ring bonded to the group, or R² and R³ form a monocyclic or polycyclic aliphatic or aromatic ring with each other. It should be noted that Ar¹ may be selected from one of the foregoing groups.

Further, in one embodiment, Ar¹ includes one of the following structural groups:

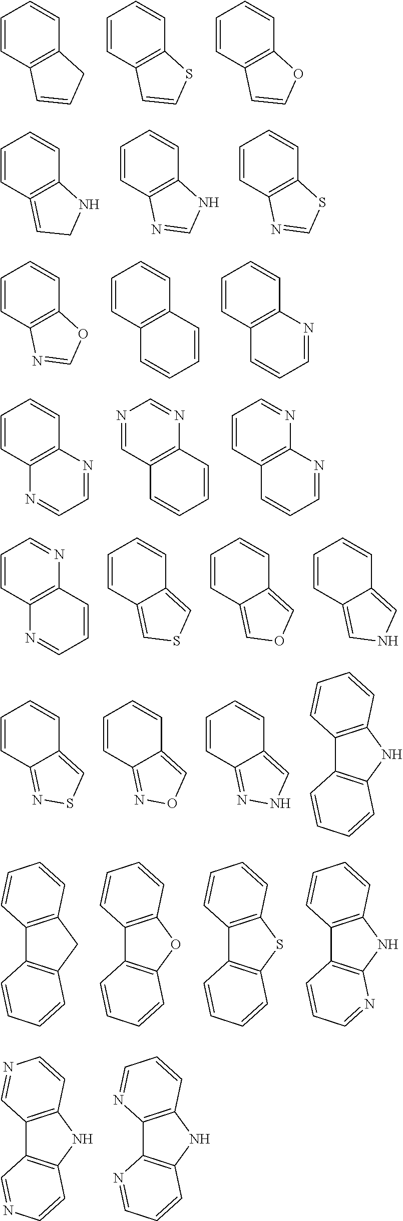

wherein H of any ring of the foregoing groups may be optionally substituted. It should be noted that Ar¹ is one selected from the foregoing groups.

Still further, Ar¹ may be selected from the group consisting of phenylbenzene, naphthalene, anthracene, phenanthrene, pyrene, pyridine, pyrimidine, triazine, fluorene, silafluorene, carbazole, dibenzothiophene, dibenzofuran, triphenylamine, triphenylphosphanoxid, tetraphenylsilane, spirofluorene or spirosilafluorene.

In one embodiment, Ar¹ is one selected from the following structural formulas:

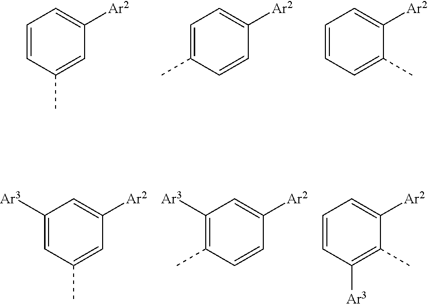

wherein Ar² and Ar³ are independently selected from an aryl group containing 5 to 60 ring atoms or a heteroaryl group containing 5 to 60 ring atoms. It should be noted that the intermediate benzene rings in Ar² and Ar³ may be partially or completely deuterated.

In one embodiment, Ar² and Ar³ independently include one or more of the following chemical formulas:

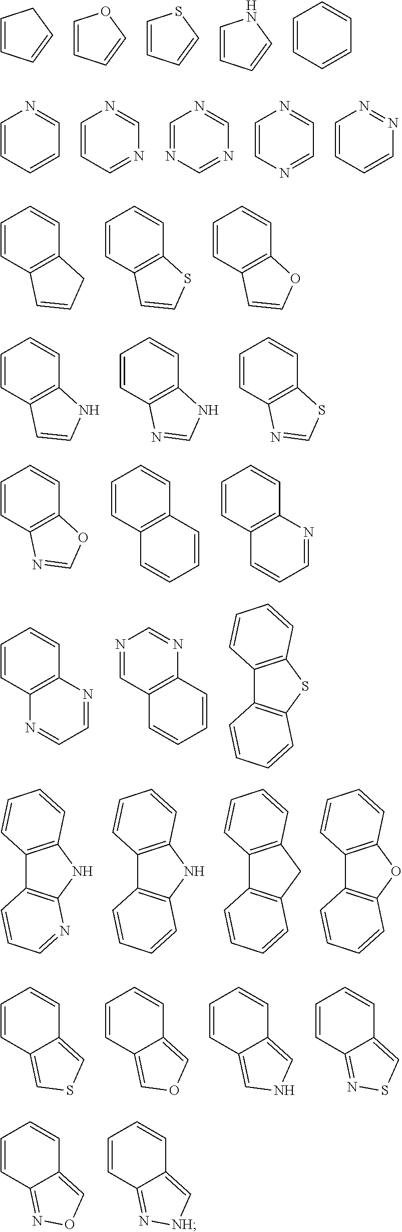

wherein H in any one of the foregoing chemical formulas may be optionally substituted. Further, Ar² and Ar³ may be independently selected from the foregoing groups.

Further, Ar² and Ar³ may independently include one or more of the following chemical formulas:

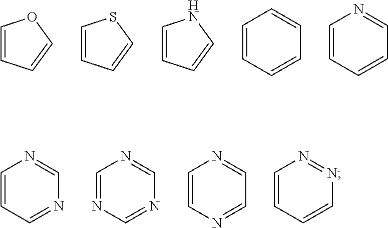

wherein H in any of the foregoing chemical formulas may be optionally substituted. Ar² and Ar³ may be independently selected from the foregoing groups.

Further, Ar² and Ar³ may be independently selected from benzene or derivatives thereof.

In one embodiment, the organic compound is selected from one of the structures represented by the following general formulas (2) to (8):

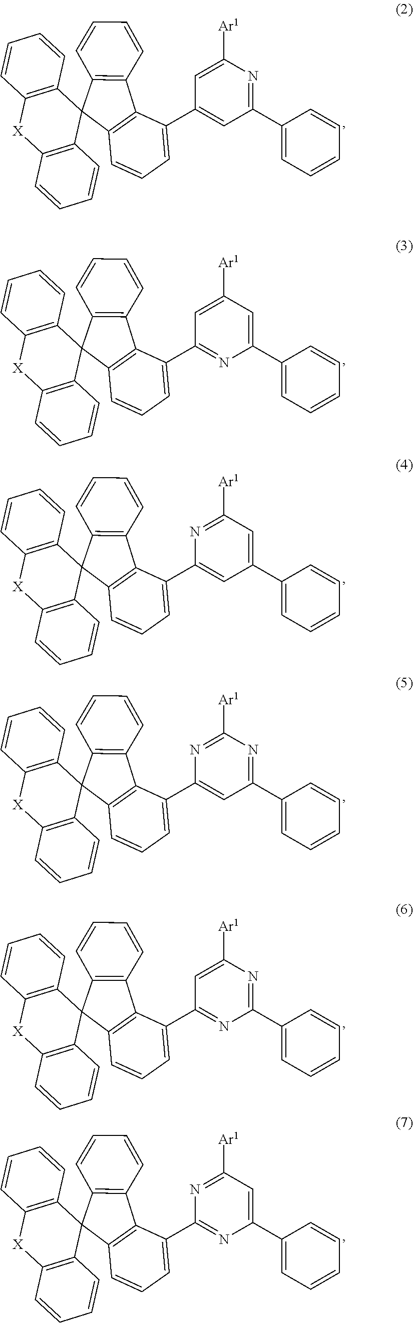

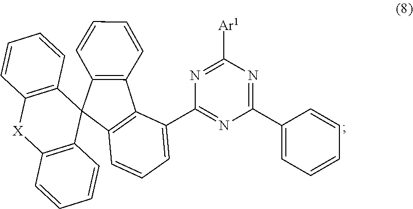

wherein X is independently selected from the group consisting of a single bond, N(R¹), C(R¹)₂, Si(R¹)₂, O, C═N(R¹), C═C(R¹)₂, P(R¹), P(═O)R¹, S, S═O or SO₂; Ar¹ is selected from an aryl group containing more than 6 ring atoms or a heteroaryl group containing more than 6 ring atoms; R¹ is selected from the group consisting of H, D, F, CN, carbonyl, sulfonyl, alkoxy, an alkyl group containing 1 to 30 carbon atoms or a cycloalkyl group containing 3 to 30 carbon atoms or an aryl group containing 5 to 60 ring atoms or a heteroaryl group containing 5 to 60 ring atoms.

In one embodiment, X shown in the general formulas (2)-(8) is selected from a single bond, N(R¹), C(R¹)₂, O or S.

In one embodiment, R¹ shown in the general formulas (2)-(8) is selected from the group consisting of H, D, an alkyl group containing 1 to 20 carbon atoms or a cycloalkyl group containing 3 to 20 carbon atoms, an aryl group containing 5 to 40 ring atoms or a heteroaryl group containing 5 to 40 ring atoms. Further, R¹ is selected from the group consisting of H, D, an alkyl group containing 1 to 10 carbon atoms or a cycloalkyl group containing 3 to 10 carbon atoms, an aryl group containing 5 to 30 ring atoms or a heteroaryl group containing 5 to 30 ring atoms. Still further, R¹ is selected from the group consisting of H, D, an alkyl group containing 1 to 4 carbon atoms or a cycloalkyl group containing 3 to 6 carbon atoms, an aryl group containing 5 to 18 ring atoms or a heteroaryl group containing 5 to 18 ring atoms.

In one embodiment, at least one of Ar¹, Ar² and Ar³ includes an electron-donating group. The electron-donating group may be selected from the group consisting of the following groups.

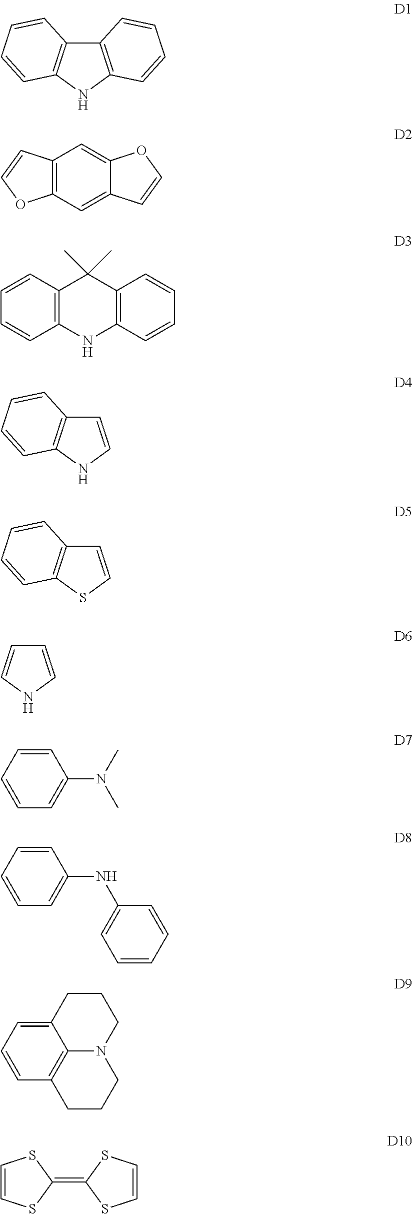

In another embodiment, at least one of Ar¹, Ar² and Ar³ includes an electron-accepting group. The electron-accepting group may be selected from F, a cyano group or a structure containing the following groups.

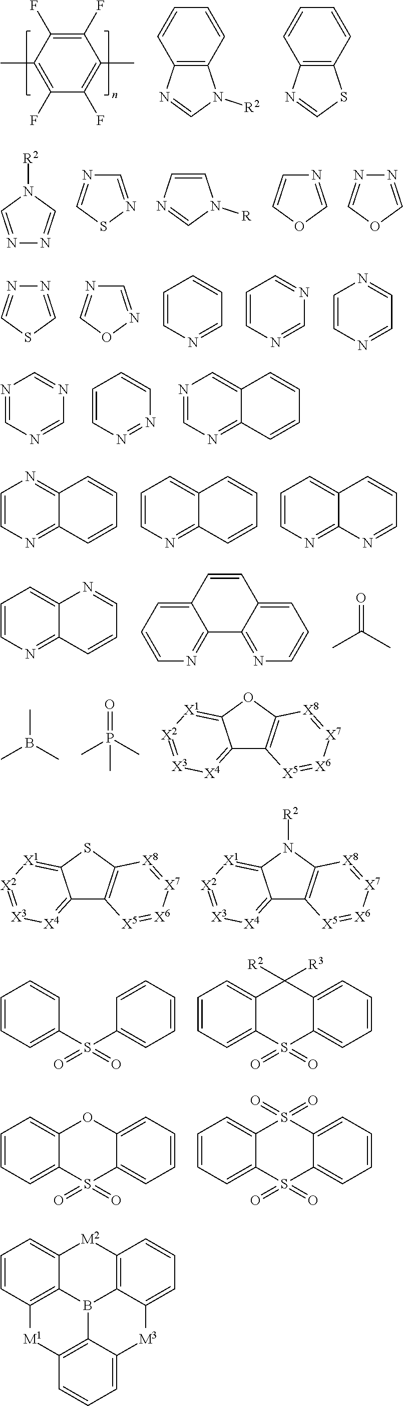

wherein n is selected from 1, 2 or 3; X¹-X⁸ are independently selected from CR or N, and at least one of X¹-X⁸ is selected from N; M¹, M² and/or M³ is not present, or M¹, M², and M³ are independently selected from N(R), C(R)₂, Si(R)₂, O, C═N(R), C═C(R)₂, P(R), P(═O)R, S, S═O or SO₂; wherein R² and R³ are one or more independently selected from the group consisting of H, D, a linear alkyl group containing 1 to 20 C atoms, a linear alkoxy group containing 1 to 20 C atoms, a linear thioalkoxy group containing 1 to 20 C atoms, a branched alkyl group containing 3 to 20 C atoms or a cyclic alkyl group containing 3 to 20 C atoms, a branched alkoxy containing 3 to 20 C atoms or a cyclic alkoxy containing 3 to 20 C atoms, a branched thioalkoxy group containing 3 to 20 C atoms or a cyclic thioalkoxy group containing 3 to 20 C atoms, a branched silyl group containing 3 to 20 C atoms or a cyclic silyl group containing 3 to 20 C atoms, a substituted keto group containing 1 to 20 C atoms, an alkoxycarbonyl group containing 2 to 20 C atoms, an aryloxycarbonyl group containing 7 to 20 C atoms, a cyano group, a carbamoyl group, a haloformyl group (—C(═O)—X, wherein X is selected from halogen atoms), a formyl group (—C(═O)—H), an isocyano group, an isocyanate group, a thiocyanate group, an isothiocyanate group, a hydroxyl group, a nitryl group, a CF₃ group, Cl, Br, F, a crosslinkable group, a substituted or non-substituted aromatic or heteroaromatic ring system containing 5 to 40 ring atoms and an aryloxy group containing 5 to 40 ring atoms or a heteroaryloxy group containing 5 to 40 ring atoms, wherein at least one of R² and R¹ forms a monocyclic or polycyclic aliphatic or aromatic ring with a ring bonded to the groups, or R² and R³ form a monocyclic or polycyclic aliphatic or aromatic ring with each other.

It should be noted that the electron-accepting group may be selected from F, a cyano group or any of the foregoing groups. Further, the absence of M¹, M² and/or M³ refers to that the adjacent two benzene rings are not connected by a bond.

In other embodiments, at least one of Ar¹, Ar² and Ar¹ includes an electron-donating group, and at least one of Ar¹, Ar² and Ar³ includes an electron-accepting group.

The foregoing organic compound can be used as an organic functional material for an organic electronic device. Organic functional materials are classified as a hole injection material (HIM), a hole transporting material (HTM), an electron transporting material (ETM), an electron injection material (EIM), an electron blocking material (EBM), a hole blocking material (HBM), an emitter and a host material. The organic compound can be used as a host material, an electron transporting material or a hole transporting material. Further, the organic compound can be used as a phosphorescent host material.

When the organic compound is used as a phosphorescent host material, the organic compound must have a proper triplet energy level. In one embodiment, the organic compound has a T₁ greater than or equal to 2.2 eV; wherein T₁ represents the first triplet excited state of the organic compound. Further, the T₁ of the organic compound is greater than or equal to 2.2 eV, in some embodiments, the T₁ of the organic compound is greater than or equal to 2.4 eV, in some embodiments, the T₁ of the organic compound is greater than or equal to 2.5 eV, in another embodiment, the T₁ of the organic compound is greater than or equal to 2.6 eV, and in yet another embodiment, the T₁ of the organic compound is greater than or equal to 2.7 eV.

When the organic compound is used as a phosphorescent host material, it is required to have high thermal stability. In one embodiment, the organic compound has a glass transition temperature T_{g} greater than or equal to 100° C. Further, T_{g} is greater than or equal to 120° C. Still further, T_{g} is greater than or equal to 140° C. Even further, T_{g} is greater than or equal to 160° C. Still further, T_{g} is greater than or equal to 180° C.

In one embodiment, the organic compound facilitates the property of thermally excited delayed fluorescence (TADF). According to the principle of TADF material (please refer to Adachi et al., Nature Vol 492, 234, (2012)), when the (S₁-T₁) of an organic compound is small enough, the triplet excitons of the organic compound can be converted to singlet excitons by internal reversion, thereby achieving efficient illumination. In general, TADF materials are obtained by connecting electron-donating groups (Donors) to electron-deficient or electron-accepting groups (acceptors), i.e., having a distinct D-A structure. Among them, (S₁-T₁) represents an energy level difference between the first triplet excited state T₁ of the organic compound and the first singlet excited state S₁ of the organic compound.

In one embodiment, the organic compound has (S₁-T₁) less than or equal to 0.30 eV, further less than or equal to 0.25 eV, still further less than or equal to 0.20 eV, even further less than or equal to 0.15 eV, and still further less than or equal to 0.10 eV.

Specific examples of the organic compound are listed below, but are not limited thereto:

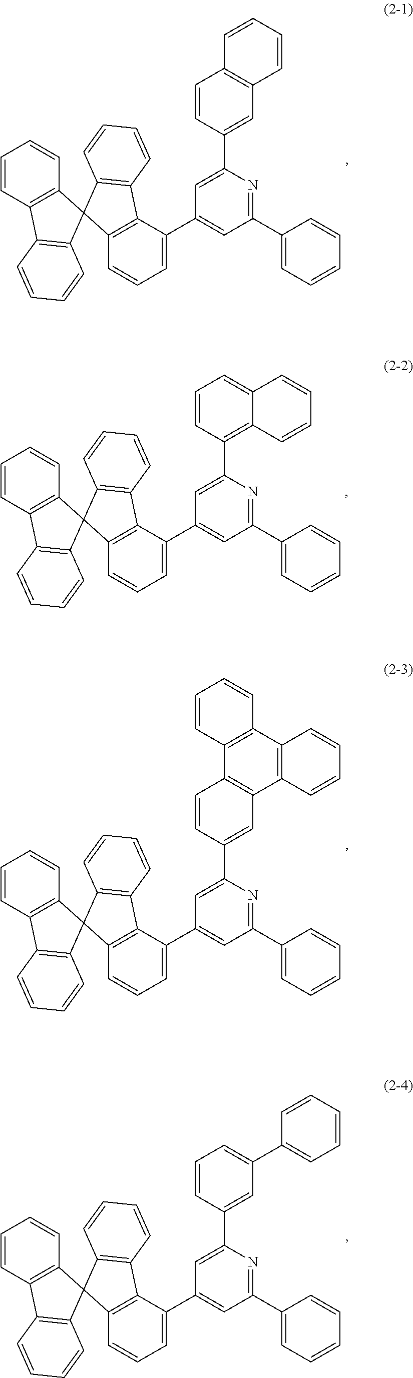 [SMILES: c1ccc(-c2cc(-c3cccc4c3-c3ccccc3C43c4ccccc4-c4ccccc43)cc(-c3ccc4ccccc4c3)n2)cc1 ; c1ccc(-c2cc(-c3cccc4c3-c3ccccc3C43c4ccccc4-c4ccccc43)cc(-c3cccc4ccccc34)n2)cc1 ; c1ccc(-c2cc(-c3cccc4c3-c3ccccc3C43c4ccccc4-c4ccccc43)cc(-c3ccc4c5ccccc5c5ccccc5c4c3)n2)cc1 ; c1ccc(-c2cccc(-c3cc(-c4cccc5c4-c4ccccc4C54c5ccccc5-c5ccccc54)cc(-c4ccccc4)n3)c2)cc1]

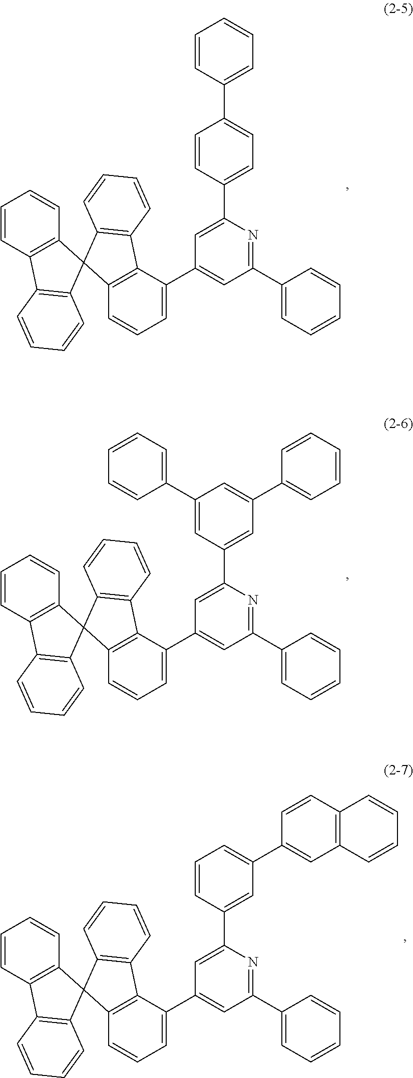 [SMILES: c1ccc(-c2ccc(-c3cc(-c4cccc5c4-c4ccccc4C54c5ccccc5-c5ccccc54)cc(-c4ccccc4)n3)cc2)cc1 ; c1ccc(-c2cc(-c3ccccc3)cc(-c3cc(-c4cccc5c4-c4ccccc4C54c5ccccc5-c5ccccc54)cc(-c4ccccc4)n3)c2)cc1 ; c1ccc(-c2cc(-c3cccc4c3-c3ccccc3C43c4ccccc4-c4ccccc43)cc(-c3cccc(-c4ccc5ccccc5c4)c3)n2)cc1]

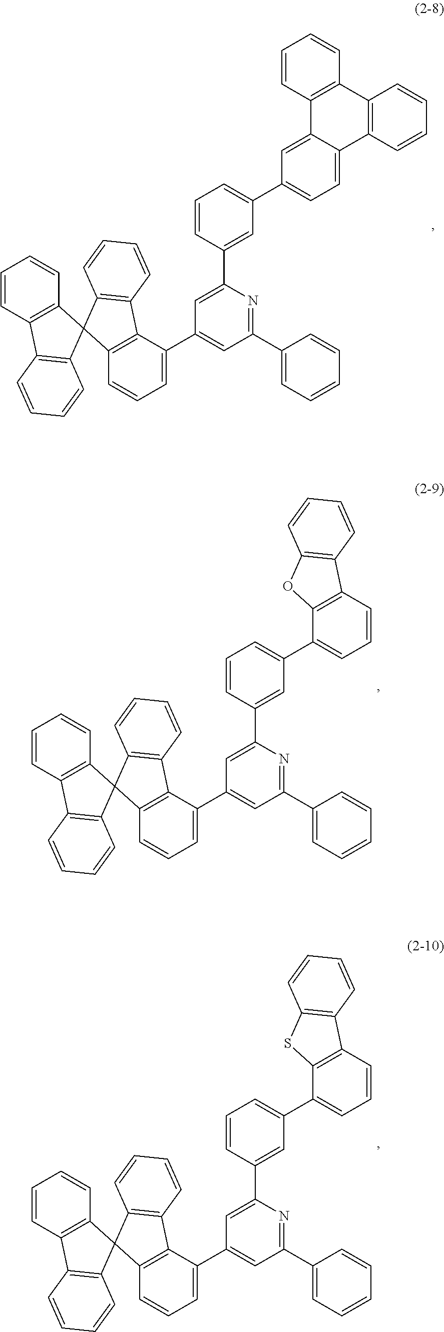 [SMILES: c1ccc(-c2cc(-c3cccc4c3-c3ccccc3C43c4ccccc4-c4ccccc43)cc(-c3cccc(-c4ccc5c6ccccc6c6ccccc6c5c4)c3)n2)cc1 ; c1ccc(-c2cc(-c3cccc4c3-c3ccccc3C43c4ccccc4-c4ccccc43)cc(-c3cccc(-c4cccc5c4oc4ccccc45)c3)n2)cc1 ; c1ccc(-c2cc(-c3cccc4c3-c3ccccc3C43c4ccccc4-c4ccccc43)cc(-c3cccc(-c4cccc5c4sc4ccccc45)c3)n2)cc1]

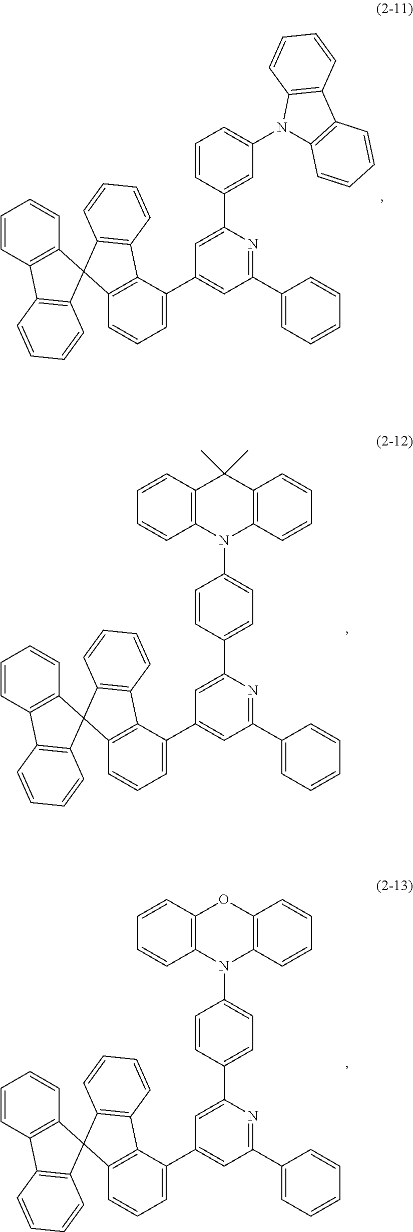 [SMILES: c1ccc(-c2cc(-c3cccc4c3-c3ccccc3C43c4ccccc4-c4ccccc43)cc(-c3cccc(-n4c5ccccc5c5ccccc54)c3)n2)cc1 ; CC1(C)c2ccccc2N(c2ccc(-c3cc(-c4cccc5c4-c4ccccc4C54c5ccccc5-c5ccccc54)cc(-c4ccccc4)n3)cc2)c2ccccc21 ; c1ccc(-c2cc(-c3cccc4c3-c3ccccc3C43c4ccccc4-c4ccccc43)cc(-c3ccc(N4c5ccccc5Oc5ccccc54)cc3)n2)cc1]

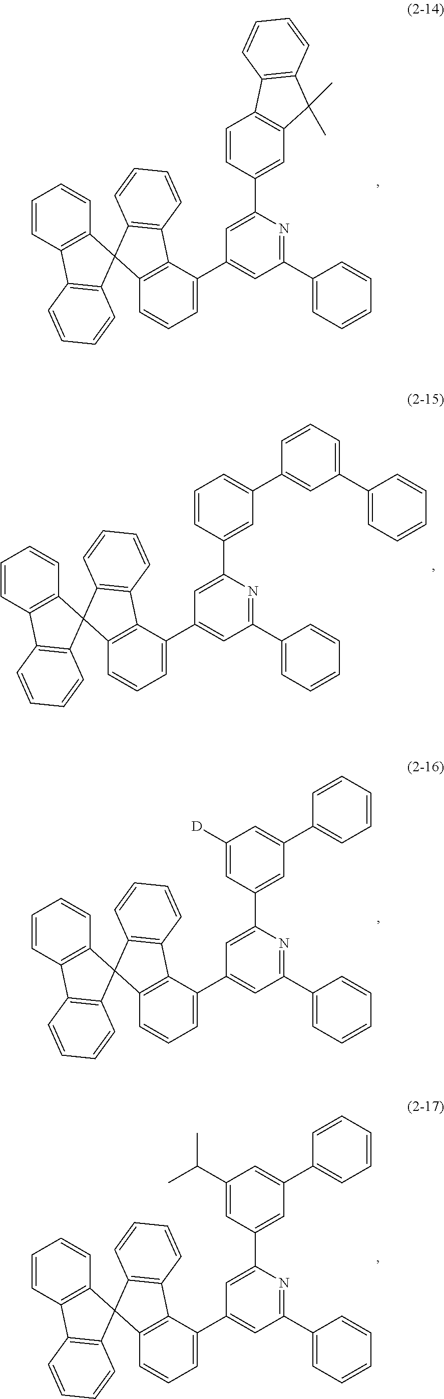 [SMILES: CC1(C)c2ccccc2-c2ccc(-c3cc(-c4cccc5c4-c4ccccc4C54c5ccccc5-c5ccccc54)cc(-c4ccccc4)n3)cc21 ; c1ccc(-c2cccc(-c3cccc(-c4cc(-c5cccc6c5-c5ccccc5C65c6ccccc6-c6ccccc65)cc(-c5ccccc5)n4)c3)c2)cc1 ; [2H]c1cc(-c2ccccc2)cc(-c2cc(-c3cccc4c3-c3ccccc3C43c4ccccc4-c4ccccc43)cc(-c3ccccc3)n2)c1 ; CC(C)c1cc(-c2ccccc2)cc(-c2cc(-c3cccc4c3-c3ccccc3C43c4ccccc4-c4ccccc43)cc(-c3ccccc3)n2)c1]

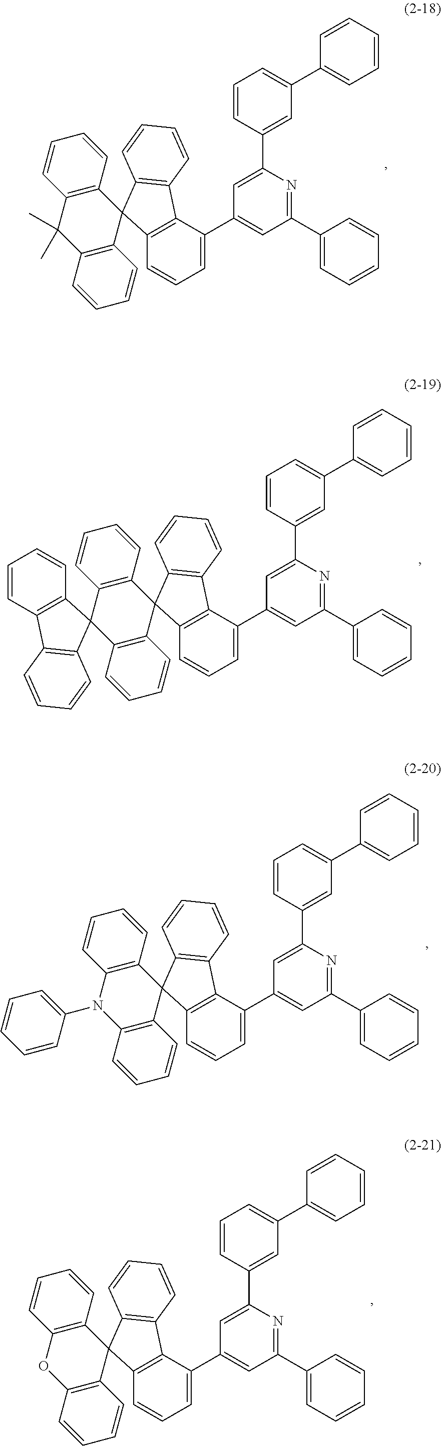 [SMILES: CC1(C)c2ccccc2C2(c3ccccc3-c3c(-c4cc(-c5ccccc5)nc(-c5cccc(-c6ccccc6)c5)c4)cccc32)c2ccccc21 ; c1ccc(-c2cccc(-c3cc(-c4cccc5c4-c4ccccc4C54c5ccccc5C5(c6ccccc6-c6ccccc65)c5ccccc54)cc(-c4ccccc4)n3)c2)cc1 ; c1ccc(-c2cccc(-c3cc(-c4cccc5c4-c4ccccc4C54c5ccccc5N(c5ccccc5)c5ccccc54)cc(-c4ccccc4)n3)c2)cc1 ; c1ccc(-c2cccc(-c3cc(-c4cccc5c4-c4ccccc4C54c5ccccc5Oc5ccccc54)cc(-c4ccccc4)n3)c2)cc1]

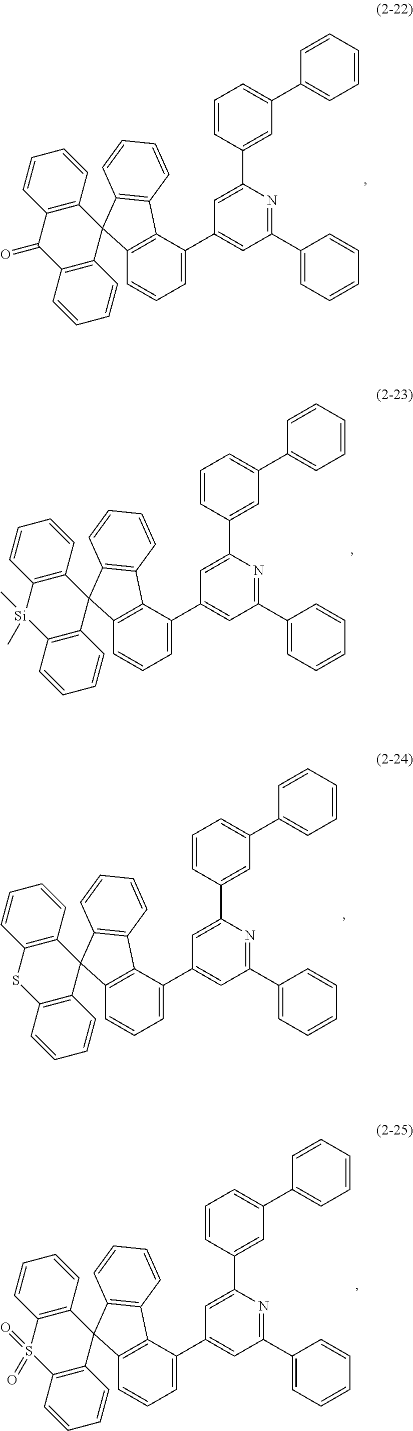 [SMILES: O=C1c2ccccc2C2(c3ccccc31)c1ccccc1-c1c(-c3cc(-c4ccccc4)nc(-c4cccc(-c5ccccc5)c4)c3)cccc12 ; C[Si]1(C)c2ccccc2C2(c3ccccc3-c3c(-c4cc(-c5ccccc5)nc(-c5cccc(-c6ccccc6)c5)c4)cccc32)c2ccccc21 ; c1ccc(-c2cccc(-c3cc(-c4cccc5c4-c4ccccc4C54c5ccccc5Sc5ccccc54)cc(-c4ccccc4)n3)c2)cc1 ; O=S1(=O)c2ccccc2C2(c3ccccc3-c3c(-c4cc(-c5ccccc5)nc(-c5cccc(-c6ccccc6)c5)c4)cccc32)c2ccccc21]

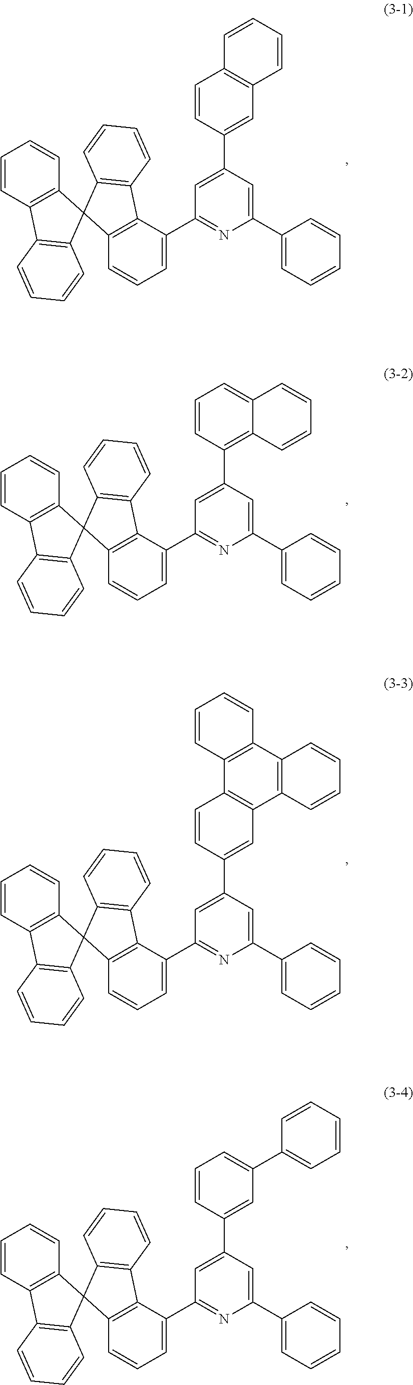 [SMILES: c1ccc(-c2cc(-c3ccc4ccccc4c3)cc(-c3cccc4c3-c3ccccc3C43c4ccccc4-c4ccccc43)n2)cc1 ; c1ccc(-c2cc(-c3cccc4ccccc34)cc(-c3cccc4c3-c3ccccc3C43c4ccccc4-c4ccccc43)n2)cc1 ; c1ccc(-c2cc(-c3ccc4c5ccccc5c5ccccc5c4c3)cc(-c3cccc4c3-c3ccccc3C43c4ccccc4-c4ccccc43)n2)cc1 ; c1ccc(-c2cccc(-c3cc(-c4ccccc4)nc(-c4cccc5c4-c4ccccc4C54c5ccccc5-c5ccccc54)c3)c2)cc1]

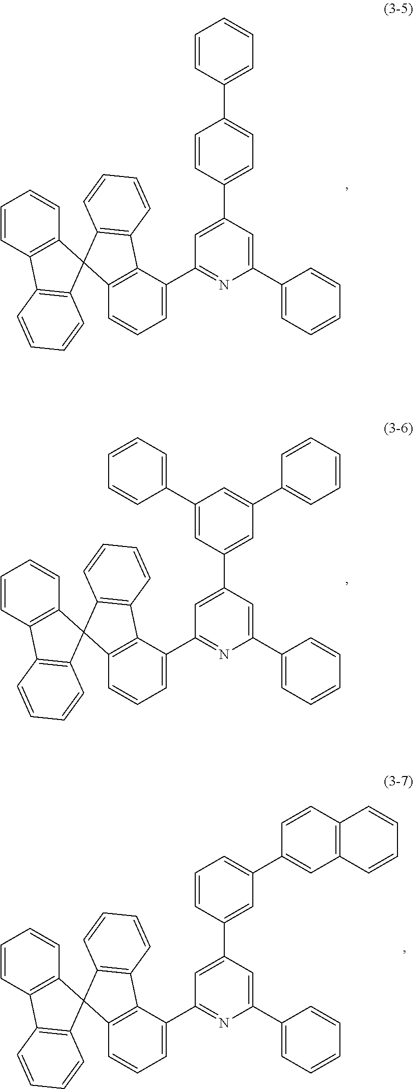 [SMILES: c1ccc(-c2ccc(-c3cc(-c4ccccc4)nc(-c4cccc5c4-c4ccccc4C54c5ccccc5-c5ccccc54)c3)cc2)cc1 ; c1ccc(-c2cc(-c3ccccc3)cc(-c3cc(-c4ccccc4)nc(-c4cccc5c4-c4ccccc4C54c5ccccc5-c5ccccc54)c3)c2)cc1 ; c1ccc(-c2cc(-c3cccc(-c4ccc5ccccc5c4)c3)cc(-c3cccc4c3-c3ccccc3C43c4ccccc4-c4ccccc43)n2)cc1]

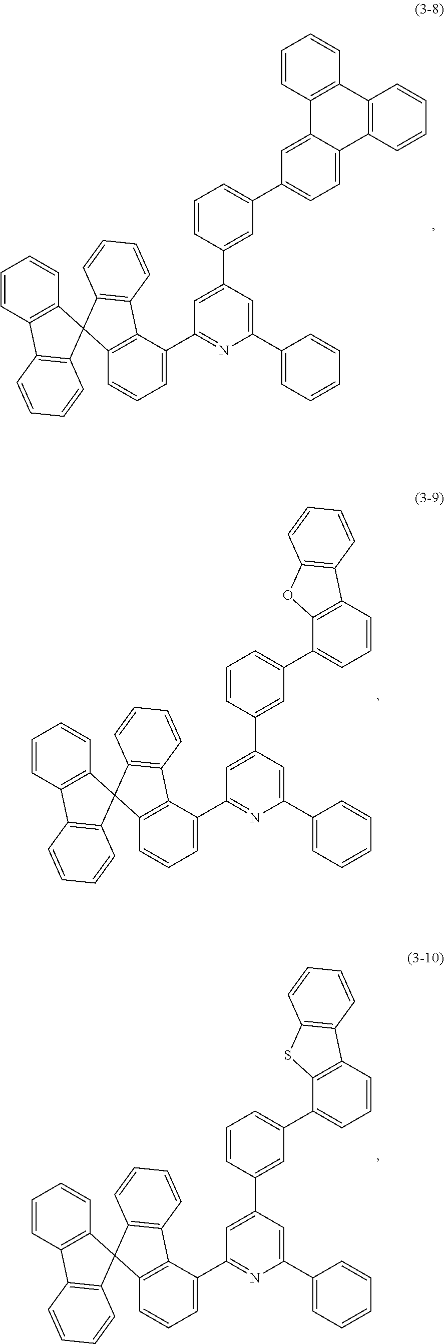 [SMILES: c1ccc(-c2cc(-c3cccc(-c4ccc5c6ccccc6c6ccccc6c5c4)c3)cc(-c3cccc4c3-c3ccccc3C43c4ccccc4-c4ccccc43)n2)cc1 ; c1ccc(-c2cc(-c3cccc(-c4cccc5c4oc4ccccc45)c3)cc(-c3cccc4c3-c3ccccc3C43c4ccccc4-c4ccccc43)n2)cc1 ; c1ccc(-c2cc(-c3cccc(-c4cccc5c4sc4ccccc45)c3)cc(-c3cccc4c3-c3ccccc3C43c4ccccc4-c4ccccc43)n2)cc1]

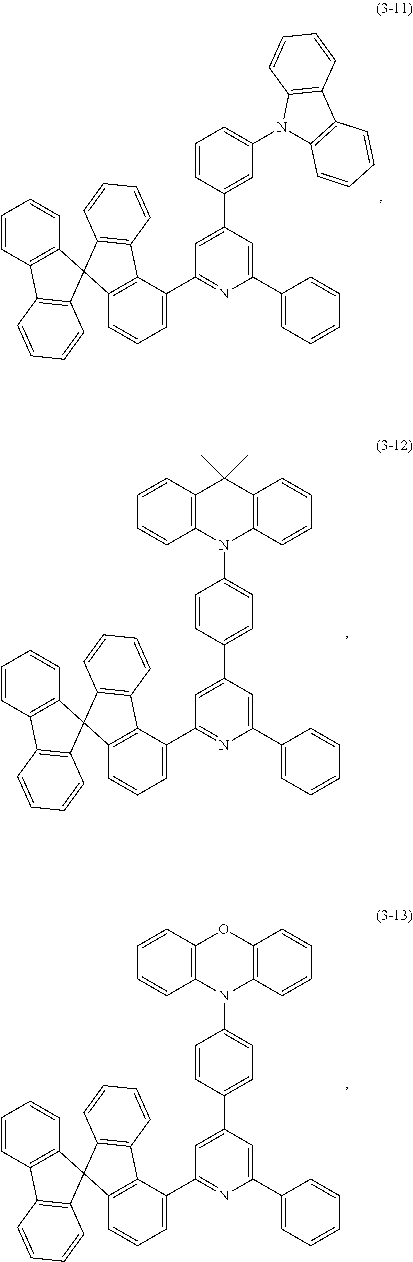 [SMILES: c1ccc(-c2cc(-c3cccc(-n4c5ccccc5c5ccccc54)c3)cc(-c3cccc4c3-c3ccccc3C43c4ccccc4-c4ccccc43)n2)cc1 ; CC1(C)c2ccccc2N(c2ccc(-c3cc(-c4ccccc4)nc(-c4cccc5c4-c4ccccc4C54c5ccccc5-c5ccccc54)c3)cc2)c2ccccc21 ; c1ccc(-c2cc(-c3ccc(N4c5ccccc5Oc5ccccc54)cc3)cc(-c3cccc4c3-c3ccccc3C43c4ccccc4-c4ccccc43)n2)cc1]

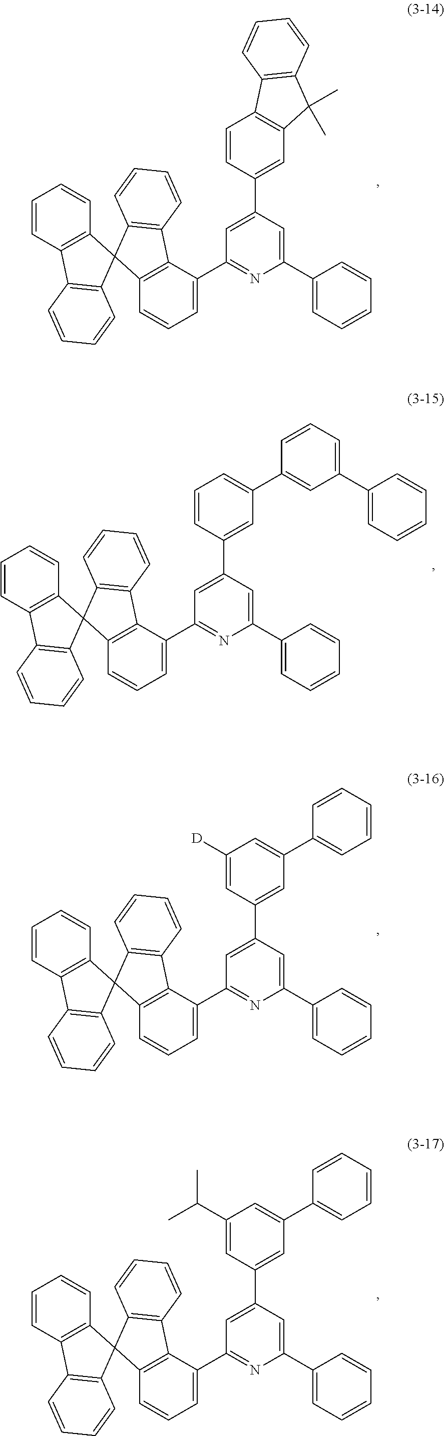 [SMILES: CC1(C)c2ccccc2-c2ccc(-c3cc(-c4ccccc4)nc(-c4cccc5c4-c4ccccc4C54c5ccccc5-c5ccccc54)c3)cc21 ; c1ccc(-c2cccc(-c3cccc(-c4cc(-c5ccccc5)nc(-c5cccc6c5-c5ccccc5C65c6ccccc6-c6ccccc65)c4)c3)c2)cc1 ; [2H]c1cc(-c2ccccc2)cc(-c2cc(-c3ccccc3)nc(-c3cccc4c3-c3ccccc3C43c4ccccc4-c4ccccc43)c2)c1 ; CC(C)c1cc(-c2ccccc2)cc(-c2cc(-c3ccccc3)nc(-c3cccc4c3-c3ccccc3C43c4ccccc4-c4ccccc43)c2)c1]

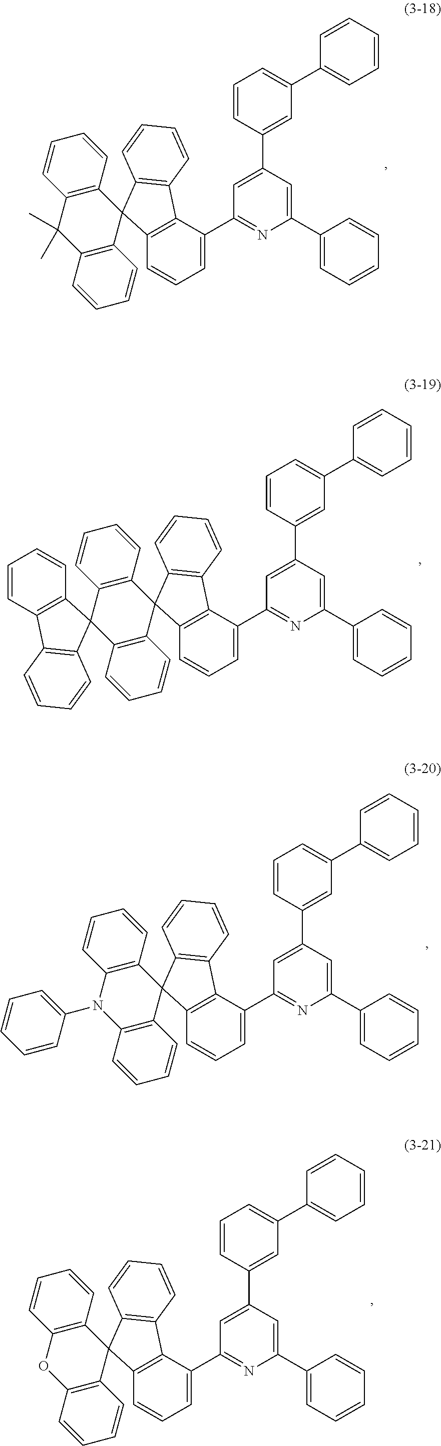 [SMILES: CC1(C)c2ccccc2C2(c3ccccc3-c3c(-c4cc(-c5cccc(-c6ccccc6)c5)cc(-c5ccccc5)n4)cccc32)c2ccccc21 ; c1ccc(-c2cccc(-c3cc(-c4ccccc4)nc(-c4cccc5c4-c4ccccc4C54c5ccccc5C5(c6ccccc6-c6ccccc65)c5ccccc54)c3)c2)cc1 ; c1ccc(-c2cccc(-c3cc(-c4ccccc4)nc(-c4cccc5c4-c4ccccc4C54c5ccccc5N(c5ccccc5)c5ccccc54)c3)c2)cc1 ; c1ccc(-c2cccc(-c3cc(-c4ccccc4)nc(-c4cccc5c4-c4ccccc4C54c5ccccc5Oc5ccccc54)c3)c2)cc1]

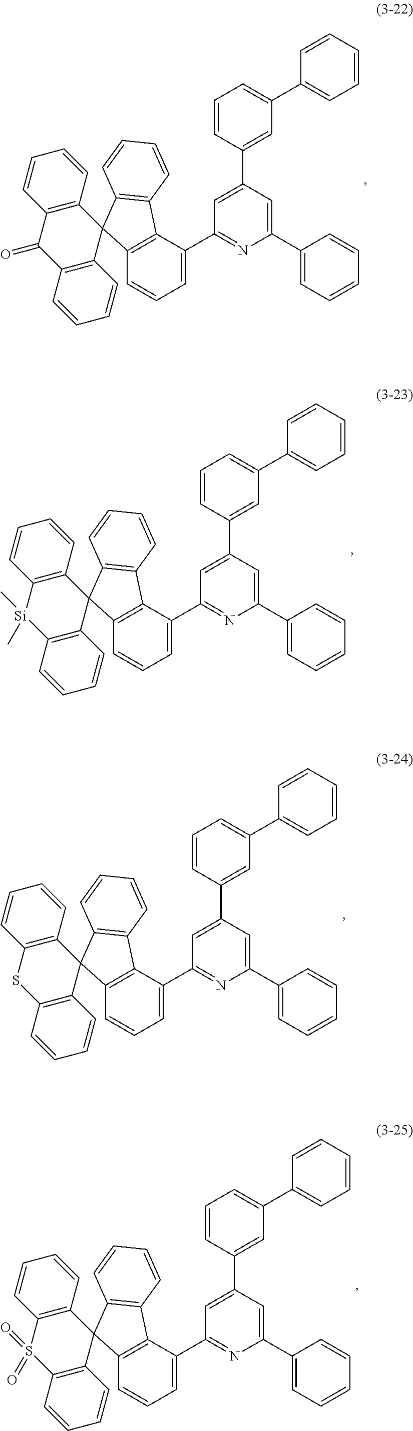 [SMILES: O=C1c2ccccc2C2(c3ccccc31)c1ccccc1-c1c(-c3cc(-c4cccc(-c5ccccc5)c4)cc(-c4ccccc4)n3)cccc12 ; C[Si]1(C)c2ccccc2C2(c3ccccc3-c3c(-c4cc(-c5cccc(-c6ccccc6)c5)cc(-c5ccccc5)n4)cccc32)c2ccccc21 ; c1ccc(-c2cccc(-c3cc(-c4ccccc4)nc(-c4cccc5c4-c4ccccc4C54c5ccccc5Sc5ccccc54)c3)c2)cc1 ; O=S1(=O)c2ccccc2C2(c3ccccc3-c3c(-c4cc(-c5cccc(-c6ccccc6)c5)cc(-c5ccccc5)n4)cccc32)c2ccccc21]

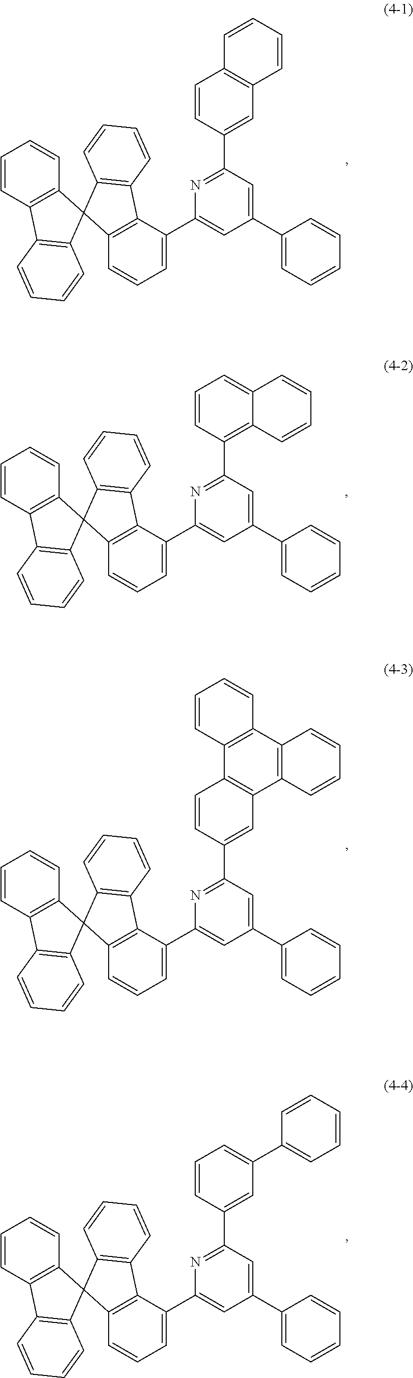 [SMILES: c1ccc(-c2cc(-c3ccc4ccccc4c3)nc(-c3cccc4c3-c3ccccc3C43c4ccccc4-c4ccccc43)c2)cc1 ; c1ccc(-c2cc(-c3cccc4c3-c3ccccc3C43c4ccccc4-c4ccccc43)nc(-c3cccc4ccccc34)c2)cc1 ; c1ccc(-c2cc(-c3ccc4c5ccccc5c5ccccc5c4c3)nc(-c3cccc4c3-c3ccccc3C43c4ccccc4-c4ccccc43)c2)cc1 ; c1ccc(-c2cccc(-c3cc(-c4ccccc4)cc(-c4cccc5c4-c4ccccc4C54c5ccccc5-c5ccccc54)n3)c2)cc1]

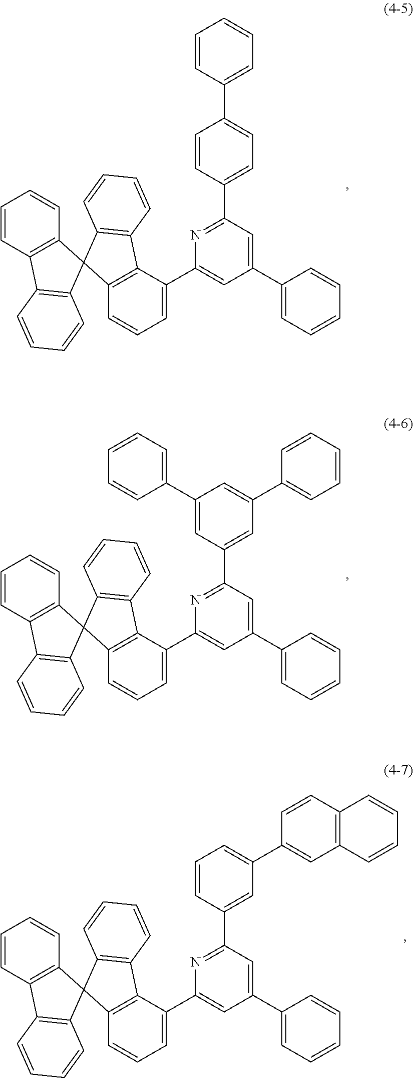 [SMILES: c1ccc(-c2ccc(-c3cc(-c4ccccc4)cc(-c4cccc5c4-c4ccccc4C54c5ccccc5-c5ccccc54)n3)cc2)cc1 ; c1ccc(-c2cc(-c3ccccc3)cc(-c3cc(-c4ccccc4)cc(-c4cccc5c4-c4ccccc4C54c5ccccc5-c5ccccc54)n3)c2)cc1 ; c1ccc(-c2cc(-c3cccc(-c4ccc5ccccc5c4)c3)nc(-c3cccc4c3-c3ccccc3C43c4ccccc4-c4ccccc43)c2)cc1]

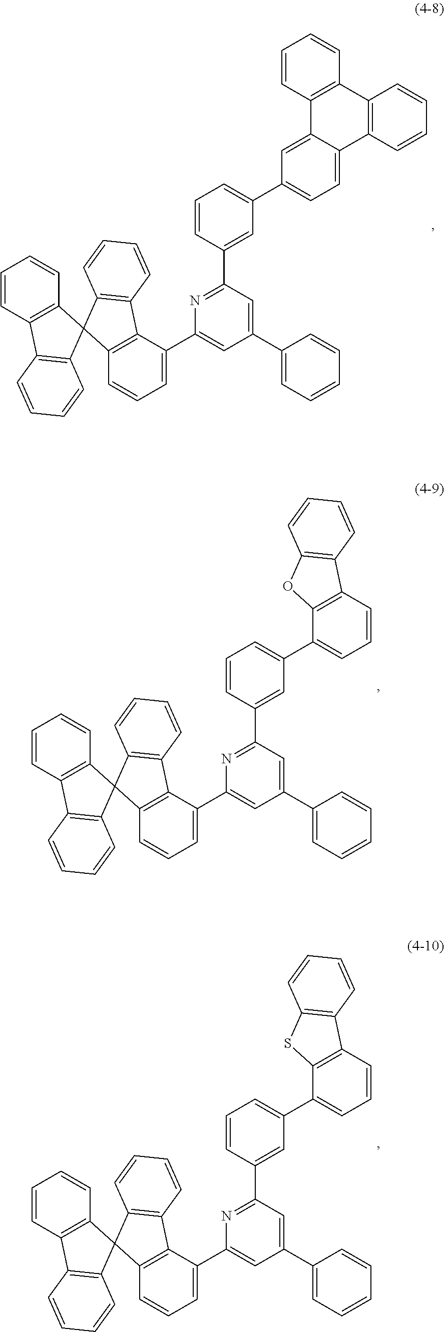 [SMILES: c1ccc(-c2cc(-c3cccc(-c4ccc5c6ccccc6c6ccccc6c5c4)c3)nc(-c3cccc4c3-c3ccccc3C43c4ccccc4-c4ccccc43)c2)cc1 ; c1ccc(-c2cc(-c3cccc(-c4cccc5c4oc4ccccc45)c3)nc(-c3cccc4c3-c3ccccc3C43c4ccccc4-c4ccccc43)c2)cc1 ; c1ccc(-c2cc(-c3cccc(-c4cccc5c4sc4ccccc45)c3)nc(-c3cccc4c3-c3ccccc3C43c4ccccc4-c4ccccc43)c2)cc1]

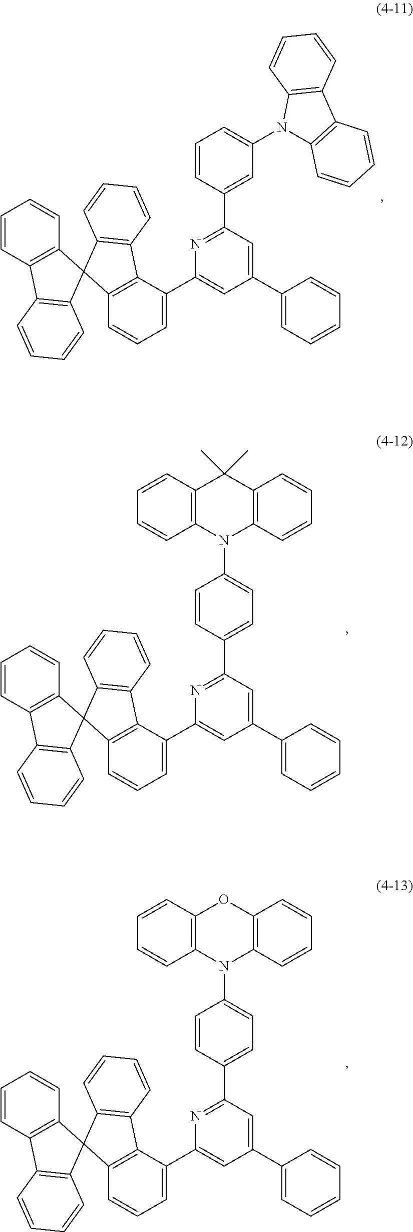 [SMILES: c1ccc(-c2cc(-c3cccc(-n4c5ccccc5c5ccccc54)c3)nc(-c3cccc4c3-c3ccccc3C43c4ccccc4-c4ccccc43)c2)cc1 ; CC1(C)c2ccccc2N(c2ccc(-c3cc(-c4ccccc4)cc(-c4cccc5c4-c4ccccc4C54c5ccccc5-c5ccccc54)n3)cc2)c2ccccc21 ; c1ccc(-c2cc(-c3ccc(N4c5ccccc5Oc5ccccc54)cc3)nc(-c3cccc4c3-c3ccccc3C43c4ccccc4-c4ccccc43)c2)cc1]

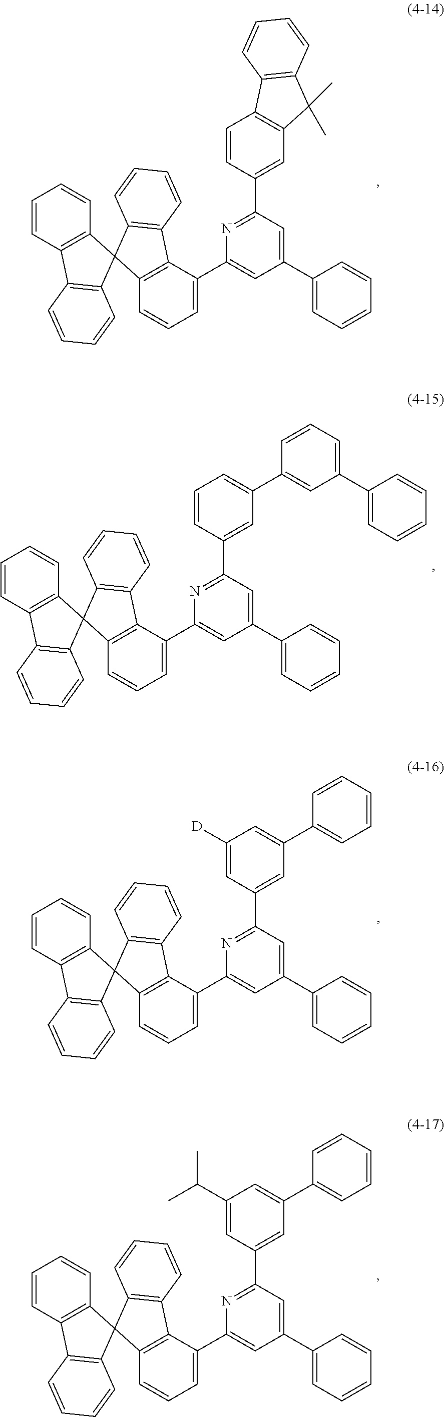 [SMILES: CC1(C)c2ccccc2-c2ccc(-c3cc(-c4ccccc4)cc(-c4cccc5c4-c4ccccc4C54c5ccccc5-c5ccccc54)n3)cc21 ; c1ccc(-c2cccc(-c3cccc(-c4cc(-c5ccccc5)cc(-c5cccc6c5-c5ccccc5C65c6ccccc6-c6ccccc65)n4)c3)c2)cc1 ; [2H]c1cc(-c2ccccc2)cc(-c2cc(-c3ccccc3)cc(-c3cccc4c3-c3ccccc3C43c4ccccc4-c4ccccc43)n2)c1 ; CC(C)c1cc(-c2ccccc2)cc(-c2cc(-c3ccccc3)cc(-c3cccc4c3-c3ccccc3C43c4ccccc4-c4ccccc43)n2)c1]

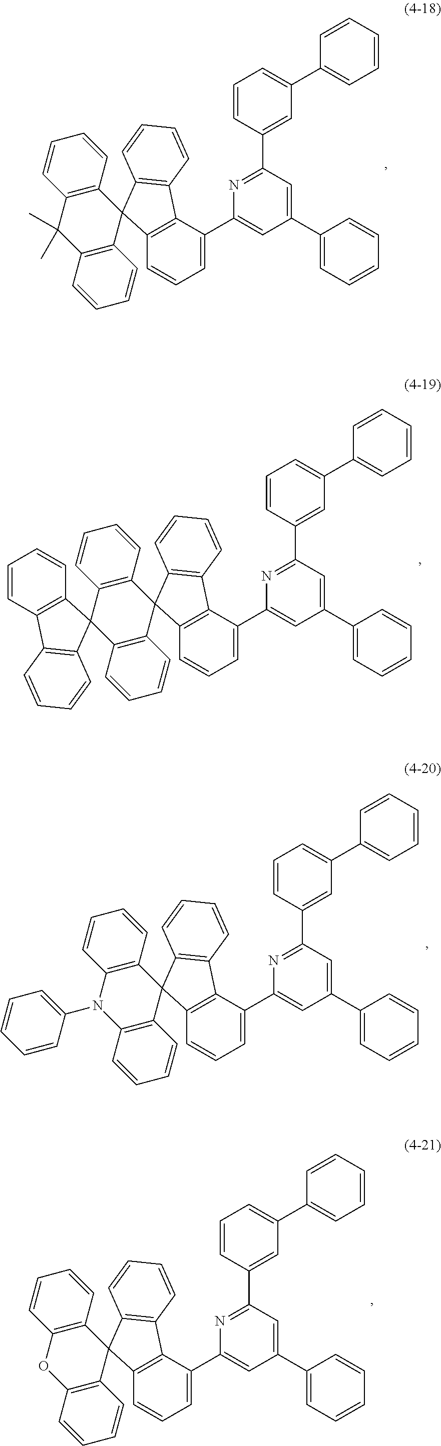 [SMILES: CC1(C)c2ccccc2C2(c3ccccc3-c3c(-c4cc(-c5ccccc5)cc(-c5cccc(-c6ccccc6)c5)n4)cccc32)c2ccccc21 ; c1ccc(-c2cccc(-c3cc(-c4ccccc4)cc(-c4cccc5c4-c4ccccc4C54c5ccccc5C5(c6ccccc6-c6ccccc65)c5ccccc54)n3)c2)cc1 ; c1ccc(-c2cccc(-c3cc(-c4ccccc4)cc(-c4cccc5c4-c4ccccc4C54c5ccccc5N(c5ccccc5)c5ccccc54)n3)c2)cc1 ; c1ccc(-c2cccc(-c3cc(-c4ccccc4)cc(-c4cccc5c4-c4ccccc4C54c5ccccc5Oc5ccccc54)n3)c2)cc1]

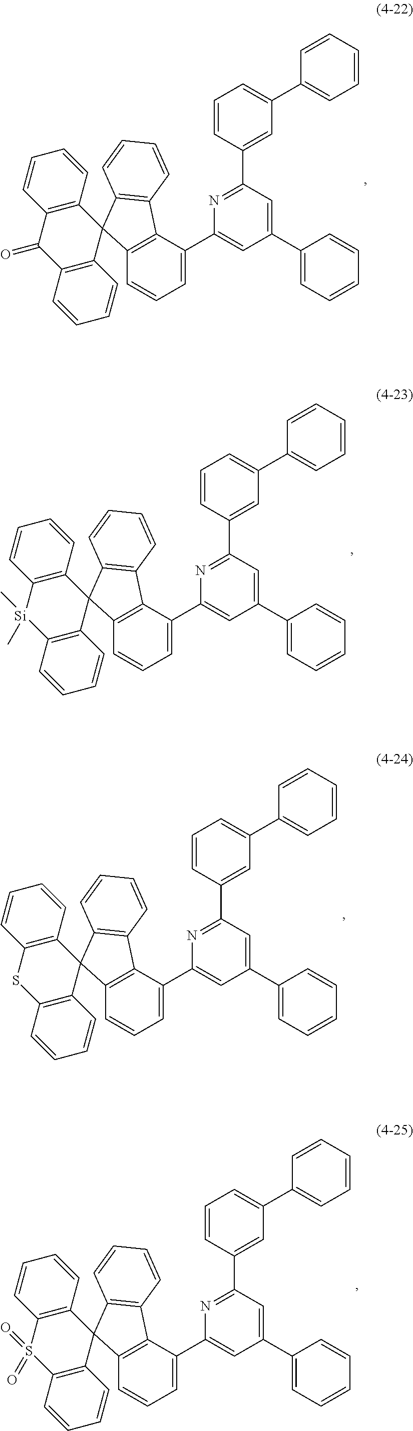 [SMILES: O=C1c2ccccc2C2(c3ccccc31)c1ccccc1-c1c(-c3cc(-c4ccccc4)cc(-c4cccc(-c5ccccc5)c4)n3)cccc12 ; C[Si]1(C)c2ccccc2C2(c3ccccc3-c3c(-c4cc(-c5ccccc5)cc(-c5cccc(-c6ccccc6)c5)n4)cccc32)c2ccccc21 ; c1ccc(-c2cccc(-c3cc(-c4ccccc4)cc(-c4cccc5c4-c4ccccc4C54c5ccccc5Sc5ccccc54)n3)c2)cc1 ; O=S1(=O)c2ccccc2C2(c3ccccc3-c3c(-c4cc(-c5ccccc5)cc(-c5cccc(-c6ccccc6)c5)n4)cccc32)c2ccccc21]

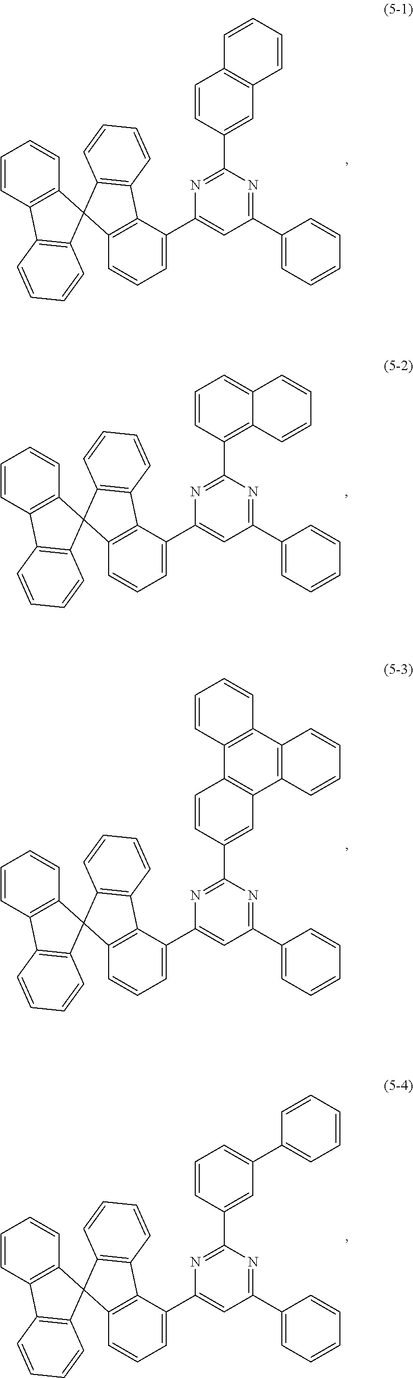 [SMILES: c1ccc(-c2cc(-c3cccc4c3-c3ccccc3C43c4ccccc4-c4ccccc43)nc(-c3ccc4ccccc4c3)n2)cc1 ; c1ccc(-c2cc(-c3cccc4c3-c3ccccc3C43c4ccccc4-c4ccccc43)nc(-c3cccc4ccccc34)n2)cc1 ; c1ccc(-c2cc(-c3cccc4c3-c3ccccc3C43c4ccccc4-c4ccccc43)nc(-c3ccc4c5ccccc5c5ccccc5c4c3)n2)cc1 ; c1ccc(-c2cccc(-c3nc(-c4ccccc4)cc(-c4cccc5c4-c4ccccc4C54c5ccccc5-c5ccccc54)n3)c2)cc1]

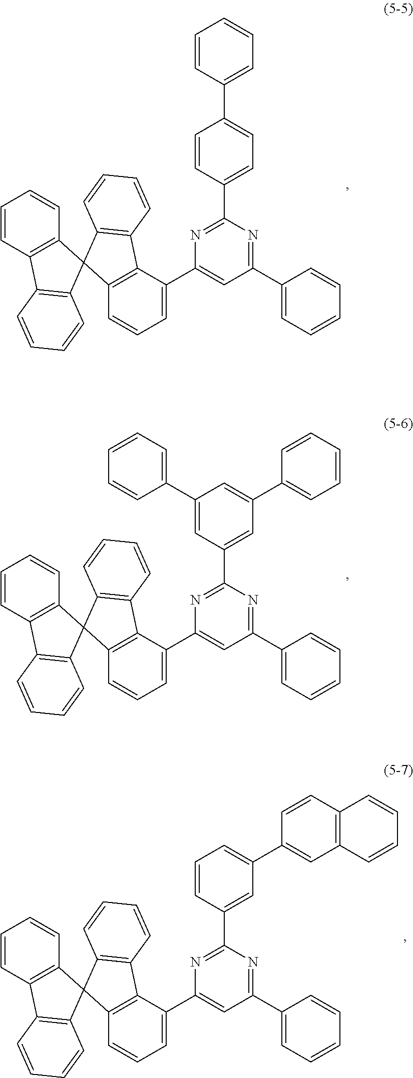 [SMILES: c1ccc(-c2ccc(-c3nc(-c4ccccc4)cc(-c4cccc5c4-c4ccccc4C54c5ccccc5-c5ccccc54)n3)cc2)cc1 ; c1ccc(-c2cc(-c3ccccc3)cc(-c3nc(-c4ccccc4)cc(-c4cccc5c4-c4ccccc4C54c5ccccc5-c5ccccc54)n3)c2)cc1 ; c1ccc(-c2cc(-c3cccc4c3-c3ccccc3C43c4ccccc4-c4ccccc43)nc(-c3cccc(-c4ccc5ccccc5c4)c3)n2)cc1]

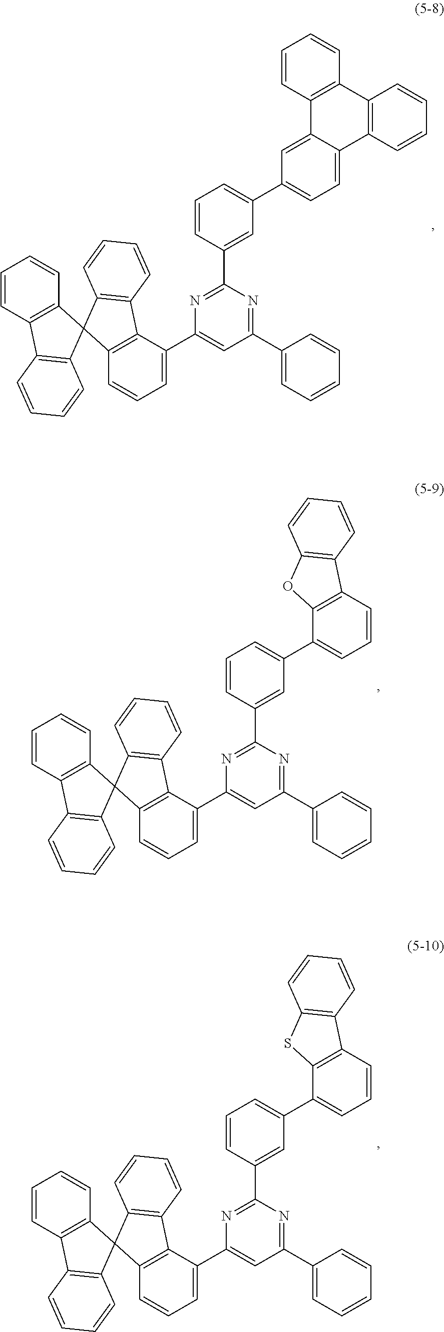 [SMILES: c1ccc(-c2cc(-c3cccc4c3-c3ccccc3C43c4ccccc4-c4ccccc43)nc(-c3cccc(-c4ccc5c6ccccc6c6ccccc6c5c4)c3)n2)cc1 ; c1ccc(-c2cc(-c3cccc4c3-c3ccccc3C43c4ccccc4-c4ccccc43)nc(-c3cccc(-c4cccc5c4oc4ccccc45)c3)n2)cc1 ; c1ccc(-c2cc(-c3cccc4c3-c3ccccc3C43c4ccccc4-c4ccccc43)nc(-c3cccc(-c4cccc5c4sc4ccccc45)c3)n2)cc1]

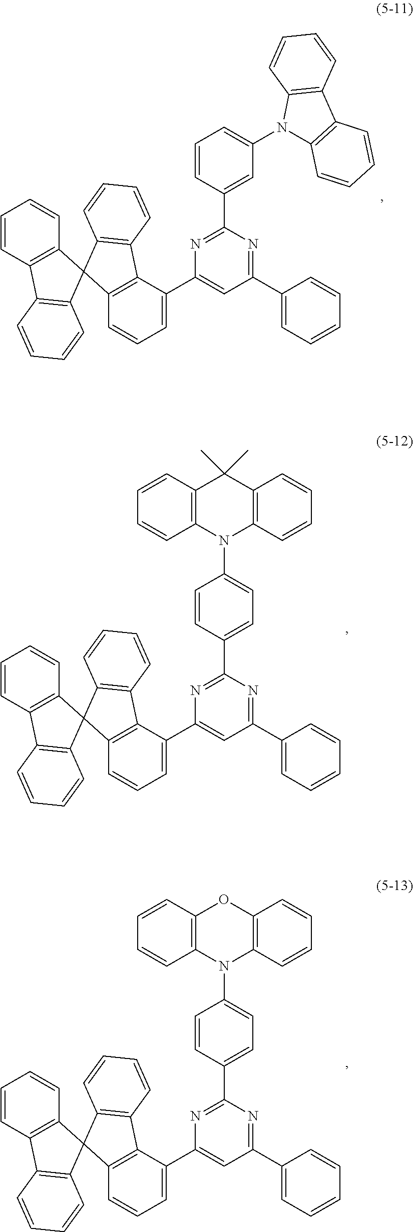 [SMILES: c1ccc(-c2cc(-c3cccc4c3-c3ccccc3C43c4ccccc4-c4ccccc43)nc(-c3cccc(-n4c5ccccc5c5ccccc54)c3)n2)cc1 ; CC1(C)c2ccccc2N(c2ccc(-c3nc(-c4ccccc4)cc(-c4cccc5c4-c4ccccc4C54c5ccccc5-c5ccccc54)n3)cc2)c2ccccc21 ; c1ccc(-c2cc(-c3cccc4c3-c3ccccc3C43c4ccccc4-c4ccccc43)nc(-c3ccc(N4c5ccccc5Oc5ccccc54)cc3)n2)cc1]

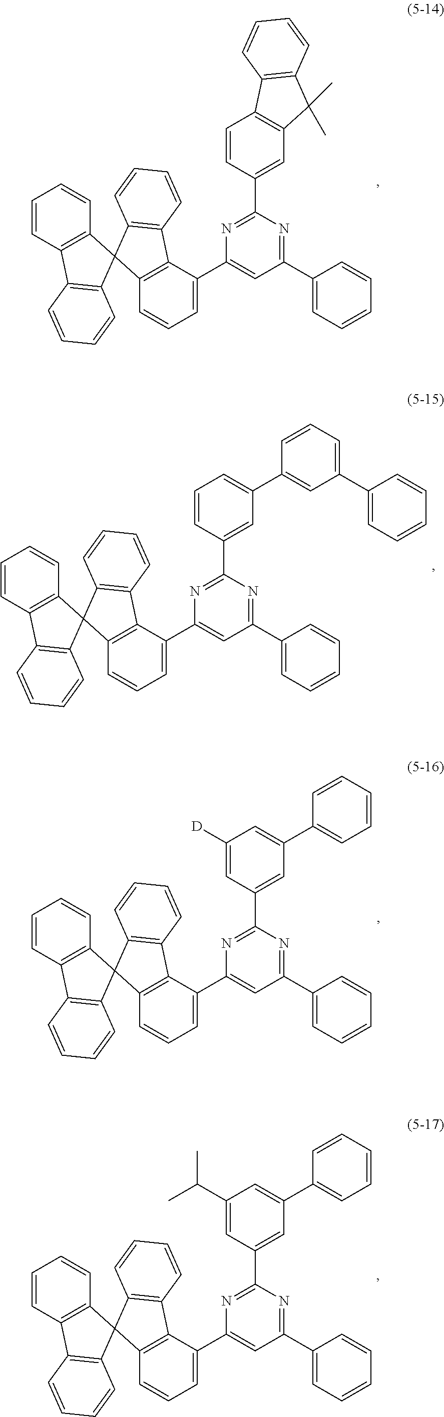 [SMILES: CC1(C)c2ccccc2-c2ccc(-c3nc(-c4ccccc4)cc(-c4cccc5c4-c4ccccc4C54c5ccccc5-c5ccccc54)n3)cc21 ; c1ccc(-c2cccc(-c3cccc(-c4nc(-c5ccccc5)cc(-c5cccc6c5-c5ccccc5C65c6ccccc6-c6ccccc65)n4)c3)c2)cc1 ; [2H]c1cc(-c2ccccc2)cc(-c2nc(-c3ccccc3)cc(-c3cccc4c3-c3ccccc3C43c4ccccc4-c4ccccc43)n2)c1 ; CC(C)c1cc(-c2ccccc2)cc(-c2nc(-c3ccccc3)cc(-c3cccc4c3-c3ccccc3C43c4ccccc4-c4ccccc43)n2)c1]

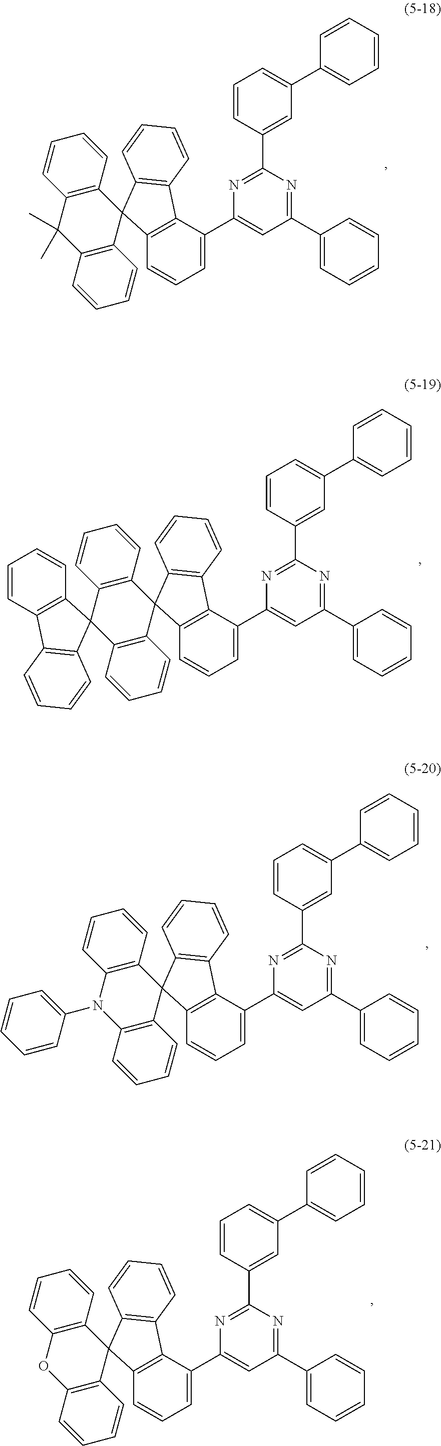 [SMILES: CC1(C)c2ccccc2C2(c3ccccc3-c3c(-c4cc(-c5ccccc5)nc(-c5cccc(-c6ccccc6)c5)n4)cccc32)c2ccccc21 ; c1ccc(-c2cccc(-c3nc(-c4ccccc4)cc(-c4cccc5c4-c4ccccc4C54c5ccccc5C5(c6ccccc6-c6ccccc65)c5ccccc54)n3)c2)cc1 ; c1ccc(-c2cccc(-c3nc(-c4ccccc4)cc(-c4cccc5c4-c4ccccc4C54c5ccccc5N(c5ccccc5)c5ccccc54)n3)c2)cc1 ; c1ccc(-c2cccc(-c3nc(-c4ccccc4)cc(-c4cccc5c4-c4ccccc4C54c5ccccc5Oc5ccccc54)n3)c2)cc1]

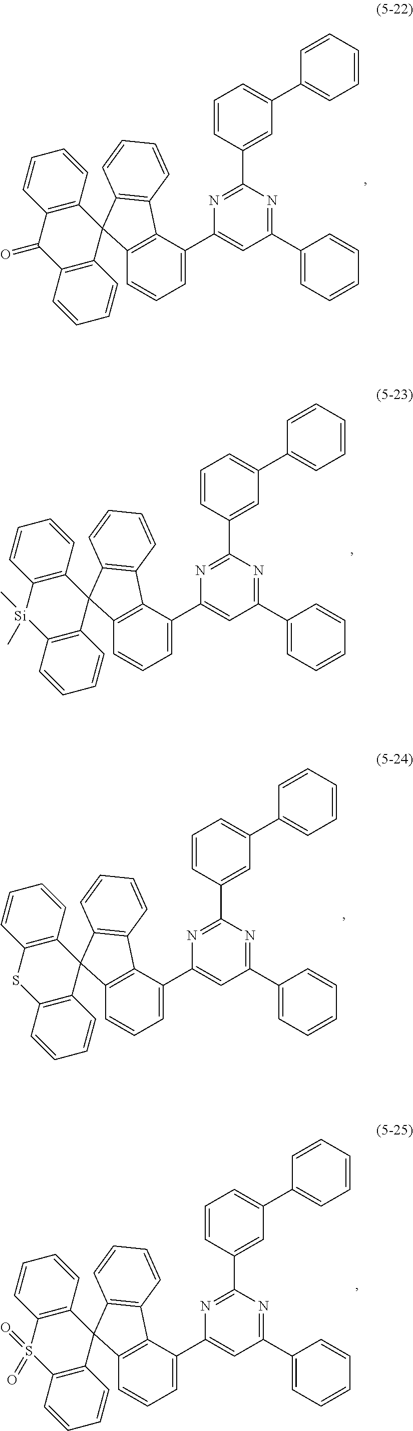 [SMILES: O=C1c2ccccc2C2(c3ccccc31)c1ccccc1-c1c(-c3cc(-c4ccccc4)nc(-c4cccc(-c5ccccc5)c4)n3)cccc12 ; C[Si]1(C)c2ccccc2C2(c3ccccc3-c3c(-c4cc(-c5ccccc5)nc(-c5cccc(-c6ccccc6)c5)n4)cccc32)c2ccccc21 ; c1ccc(-c2cccc(-c3nc(-c4ccccc4)cc(-c4cccc5c4-c4ccccc4C54c5ccccc5Sc5ccccc54)n3)c2)cc1 ; O=S1(=O)c2ccccc2C2(c3ccccc3-c3c(-c4cc(-c5ccccc5)nc(-c5cccc(-c6ccccc6)c5)n4)cccc32)c2ccccc21]

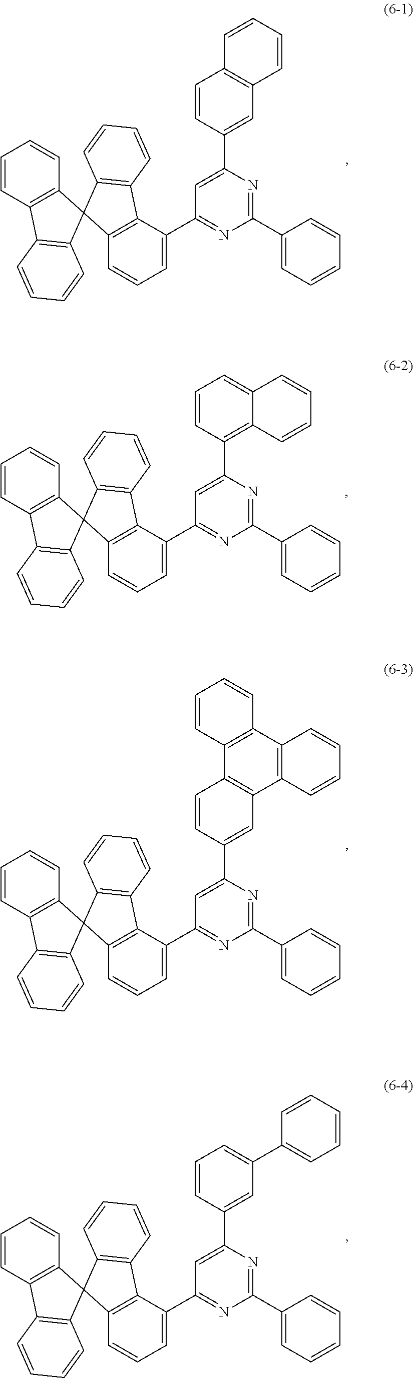 [SMILES: c1ccc(-c2nc(-c3ccc4ccccc4c3)cc(-c3cccc4c3-c3ccccc3C43c4ccccc4-c4ccccc43)n2)cc1 ; c1ccc(-c2nc(-c3cccc4c3-c3ccccc3C43c4ccccc4-c4ccccc43)cc(-c3cccc4ccccc34)n2)cc1 ; c1ccc(-c2nc(-c3ccc4c5ccccc5c5ccccc5c4c3)cc(-c3cccc4c3-c3ccccc3C43c4ccccc4-c4ccccc43)n2)cc1 ; c1ccc(-c2cccc(-c3cc(-c4cccc5c4-c4ccccc4C54c5ccccc5-c5ccccc54)nc(-c4ccccc4)n3)c2)cc1]

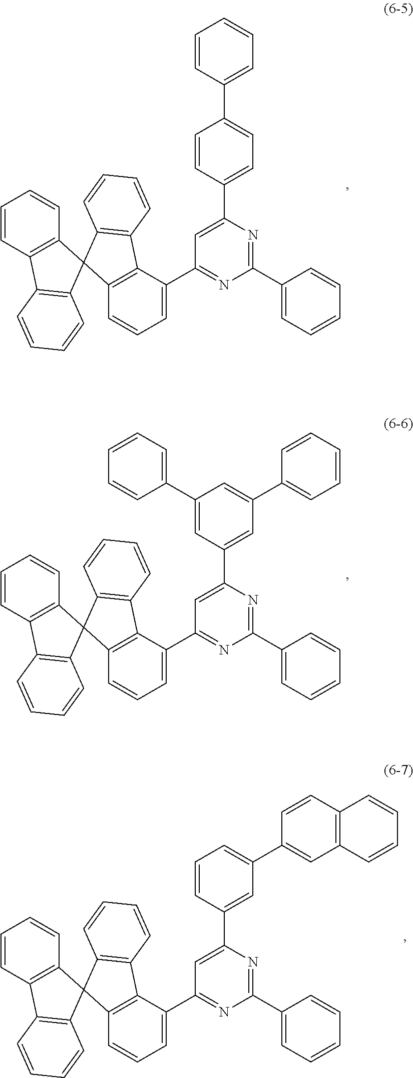 [SMILES: c1ccc(-c2ccc(-c3cc(-c4cccc5c4-c4ccccc4C54c5ccccc5-c5ccccc54)nc(-c4ccccc4)n3)cc2)cc1 ; c1ccc(-c2cc(-c3ccccc3)cc(-c3cc(-c4cccc5c4-c4ccccc4C54c5ccccc5-c5ccccc54)nc(-c4ccccc4)n3)c2)cc1 ; c1ccc(-c2nc(-c3cccc(-c4ccc5ccccc5c4)c3)cc(-c3cccc4c3-c3ccccc3C43c4ccccc4-c4ccccc43)n2)cc1]

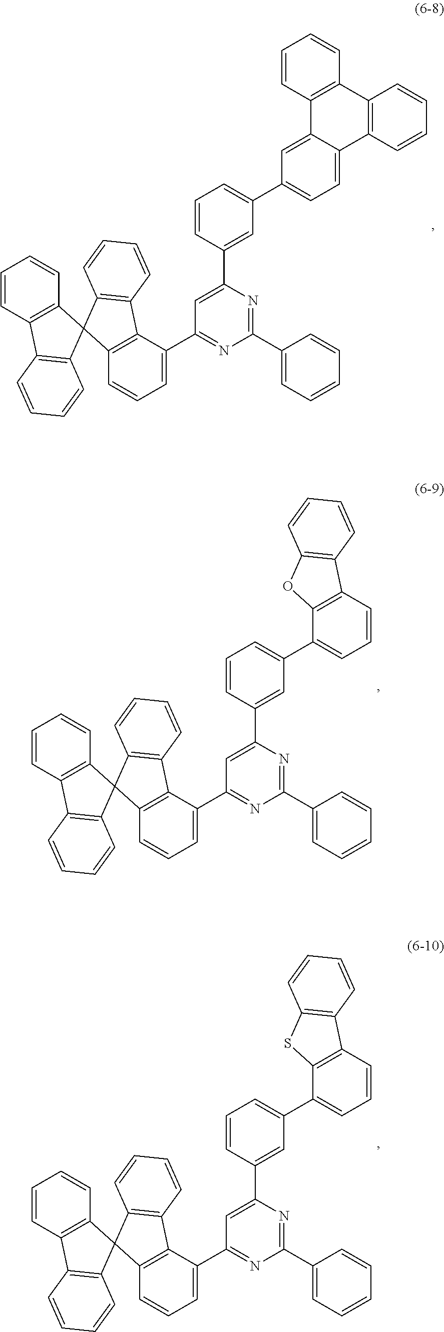 [SMILES: c1ccc(-c2nc(-c3cccc(-c4ccc5c6ccccc6c6ccccc6c5c4)c3)cc(-c3cccc4c3-c3ccccc3C43c4ccccc4-c4ccccc43)n2)cc1 ; c1ccc(-c2nc(-c3cccc(-c4cccc5c4oc4ccccc45)c3)cc(-c3cccc4c3-c3ccccc3C43c4ccccc4-c4ccccc43)n2)cc1 ; c1ccc(-c2nc(-c3cccc(-c4cccc5c4sc4ccccc45)c3)cc(-c3cccc4c3-c3ccccc3C43c4ccccc4-c4ccccc43)n2)cc1]

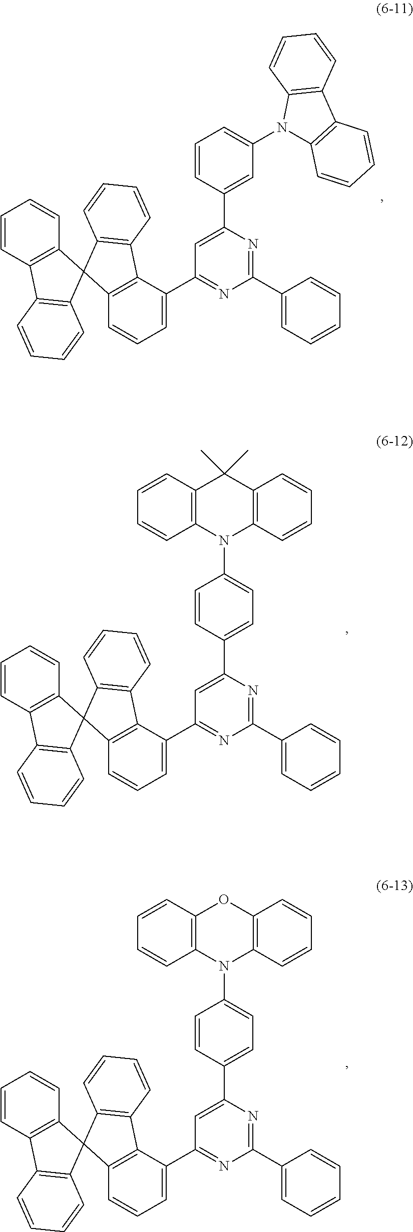 [SMILES: c1ccc(-c2nc(-c3cccc(-n4c5ccccc5c5ccccc54)c3)cc(-c3cccc4c3-c3ccccc3C43c4ccccc4-c4ccccc43)n2)cc1 ; CC1(C)c2ccccc2N(c2ccc(-c3cc(-c4cccc5c4-c4ccccc4C54c5ccccc5-c5ccccc54)nc(-c4ccccc4)n3)cc2)c2ccccc21 ; c1ccc(-c2nc(-c3ccc(N4c5ccccc5Oc5ccccc54)cc3)cc(-c3cccc4c3-c3ccccc3C43c4ccccc4-c4ccccc43)n2)cc1]

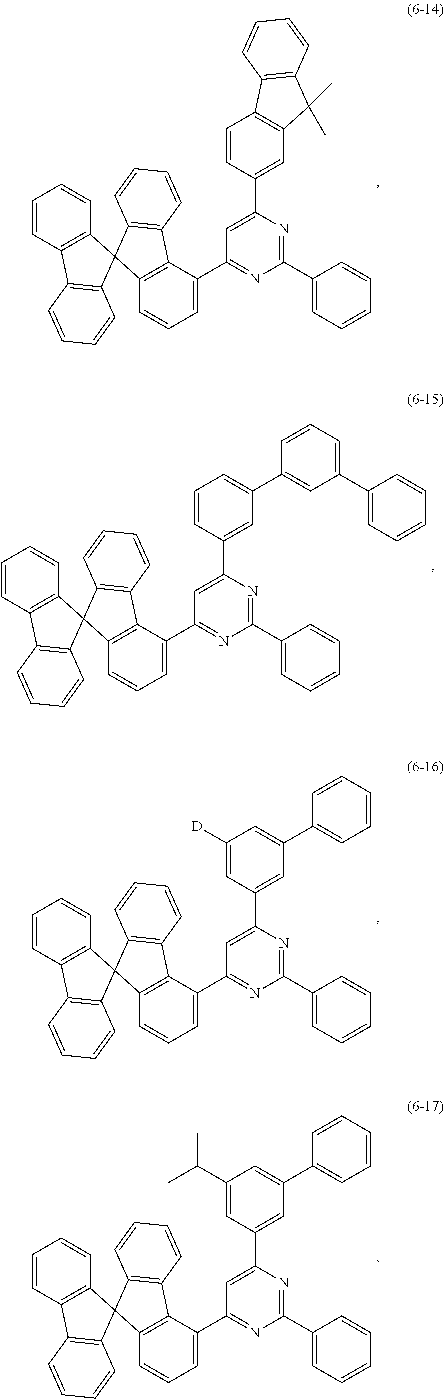 [SMILES: CC1(C)c2ccccc2-c2ccc(-c3cc(-c4cccc5c4-c4ccccc4C54c5ccccc5-c5ccccc54)nc(-c4ccccc4)n3)cc21 ; c1ccc(-c2cccc(-c3cccc(-c4cc(-c5cccc6c5-c5ccccc5C65c6ccccc6-c6ccccc65)nc(-c5ccccc5)n4)c3)c2)cc1 ; [2H]c1cc(-c2ccccc2)cc(-c2cc(-c3cccc4c3-c3ccccc3C43c4ccccc4-c4ccccc43)nc(-c3ccccc3)n2)c1 ; CC(C)c1cc(-c2ccccc2)cc(-c2cc(-c3cccc4c3-c3ccccc3C43c4ccccc4-c4ccccc43)nc(-c3ccccc3)n2)c1]

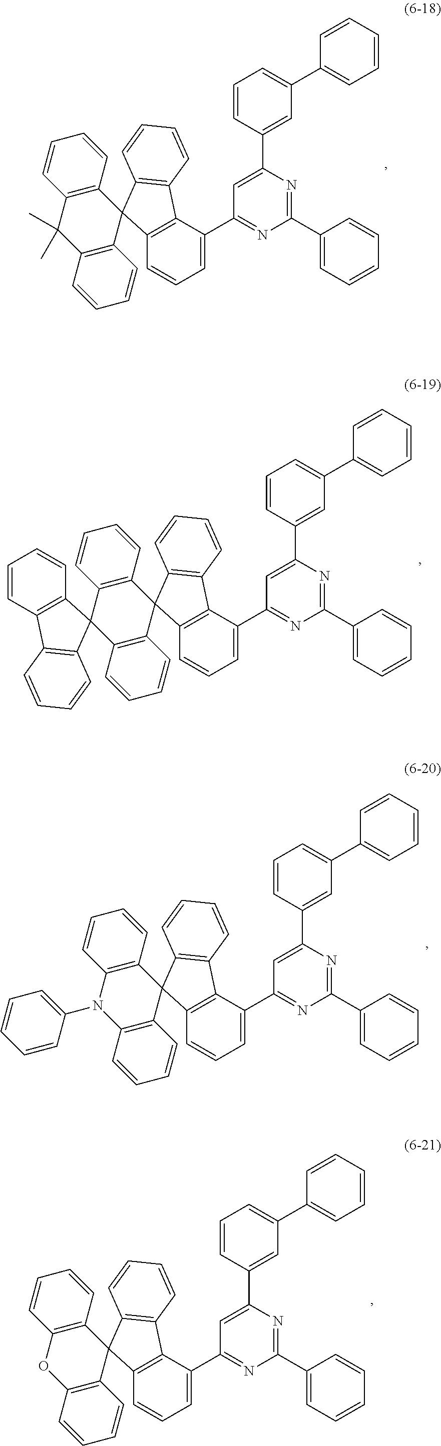 [SMILES: CC1(C)c2ccccc2C2(c3ccccc3-c3c(-c4cc(-c5cccc(-c6ccccc6)c5)nc(-c5ccccc5)n4)cccc32)c2ccccc21 ; c1ccc(-c2cccc(-c3cc(-c4cccc5c4-c4ccccc4C54c5ccccc5C5(c6ccccc6-c6ccccc65)c5ccccc54)nc(-c4ccccc4)n3)c2)cc1 ; c1ccc(-c2cccc(-c3cc(-c4cccc5c4-c4ccccc4C54c5ccccc5N(c5ccccc5)c5ccccc54)nc(-c4ccccc4)n3)c2)cc1 ; c1ccc(-c2cccc(-c3cc(-c4cccc5c4-c4ccccc4C54c5ccccc5Oc5ccccc54)nc(-c4ccccc4)n3)c2)cc1]

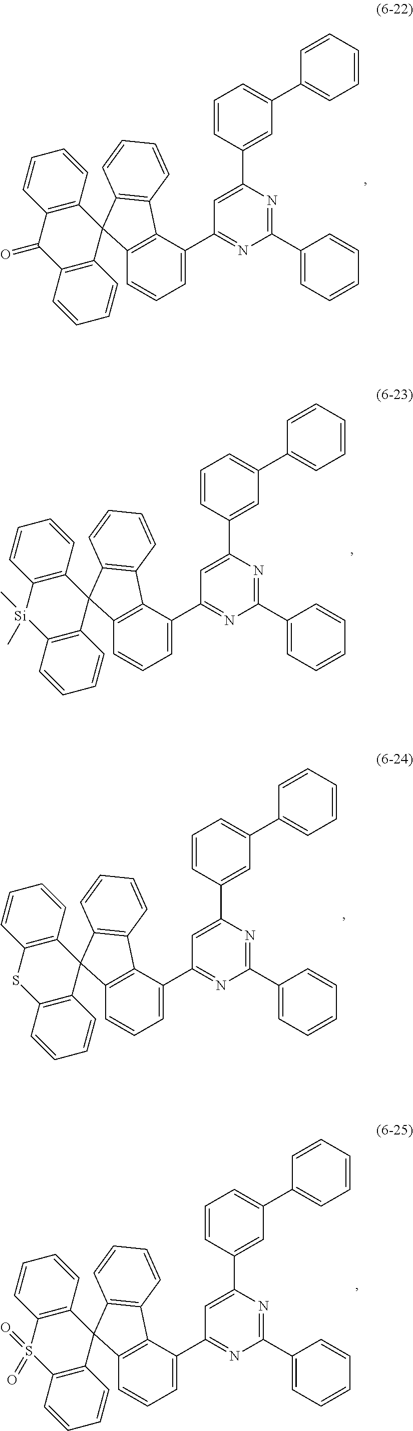 [SMILES: O=C1c2ccccc2C2(c3ccccc31)c1ccccc1-c1c(-c3cc(-c4cccc(-c5ccccc5)c4)nc(-c4ccccc4)n3)cccc12 ; C[Si]1(C)c2ccccc2C2(c3ccccc3-c3c(-c4cc(-c5cccc(-c6ccccc6)c5)nc(-c5ccccc5)n4)cccc32)c2ccccc21 ; c1ccc(-c2cccc(-c3cc(-c4cccc5c4-c4ccccc4C54c5ccccc5Sc5ccccc54)nc(-c4ccccc4)n3)c2)cc1 ; O=S1(=O)c2ccccc2C2(c3ccccc3-c3c(-c4cc(-c5cccc(-c6ccccc6)c5)nc(-c5ccccc5)n4)cccc32)c2ccccc21]

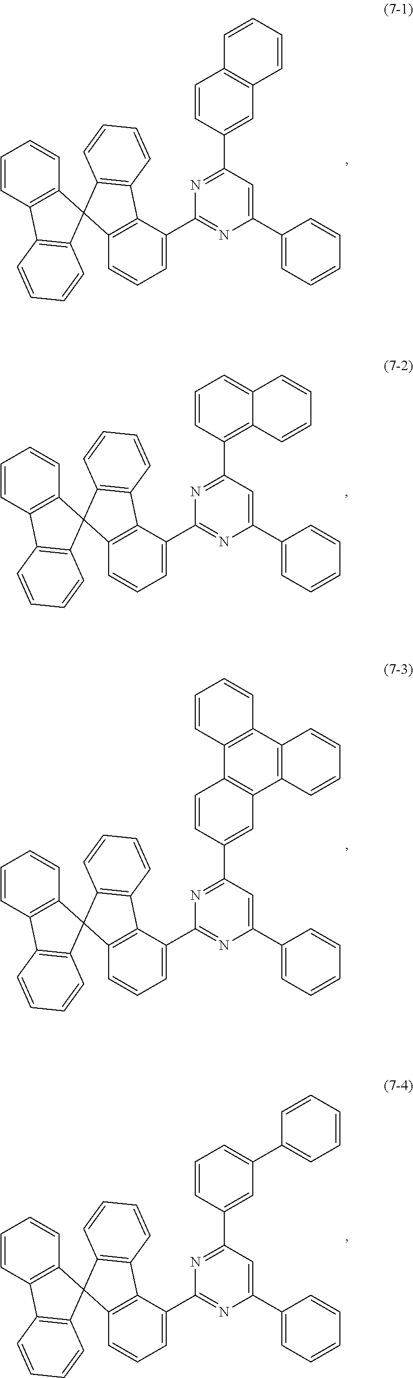 [SMILES: c1ccc(-c2cc(-c3ccc4ccccc4c3)nc(-c3cccc4c3-c3ccccc3C43c4ccccc4-c4ccccc43)n2)cc1 ; c1ccc(-c2cc(-c3cccc4ccccc34)nc(-c3cccc4c3-c3ccccc3C43c4ccccc4-c4ccccc43)n2)cc1 ; c1ccc(-c2cc(-c3ccc4c5ccccc5c5ccccc5c4c3)nc(-c3cccc4c3-c3ccccc3C43c4ccccc4-c4ccccc43)n2)cc1 ; c1ccc(-c2cccc(-c3cc(-c4ccccc4)nc(-c4cccc5c4-c4ccccc4C54c5ccccc5-c5ccccc54)n3)c2)cc1]

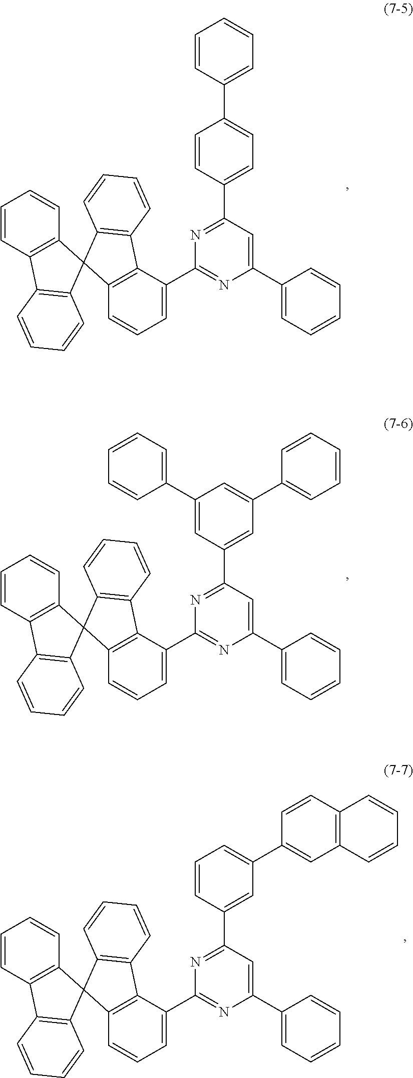 [SMILES: c1ccc(-c2ccc(-c3cc(-c4ccccc4)nc(-c4cccc5c4-c4ccccc4C54c5ccccc5-c5ccccc54)n3)cc2)cc1 ; c1ccc(-c2cc(-c3ccccc3)cc(-c3cc(-c4ccccc4)nc(-c4cccc5c4-c4ccccc4C54c5ccccc5-c5ccccc54)n3)c2)cc1 ; c1ccc(-c2cc(-c3cccc(-c4ccc5ccccc5c4)c3)nc(-c3cccc4c3-c3ccccc3C43c4ccccc4-c4ccccc43)n2)cc1]

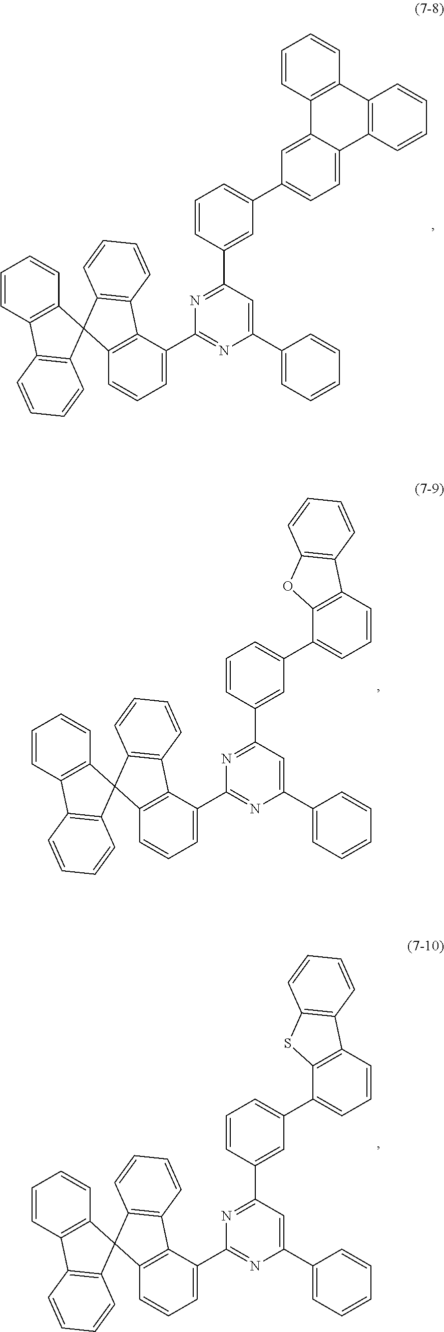 [SMILES: c1ccc(-c2cc(-c3cccc(-c4ccc5c6ccccc6c6ccccc6c5c4)c3)nc(-c3cccc4c3-c3ccccc3C43c4ccccc4-c4ccccc43)n2)cc1 ; c1ccc(-c2cc(-c3cccc(-c4cccc5c4oc4ccccc45)c3)nc(-c3cccc4c3-c3ccccc3C43c4ccccc4-c4ccccc43)n2)cc1 ; c1ccc(-c2cc(-c3cccc(-c4cccc5c4sc4ccccc45)c3)nc(-c3cccc4c3-c3ccccc3C43c4ccccc4-c4ccccc43)n2)cc1]

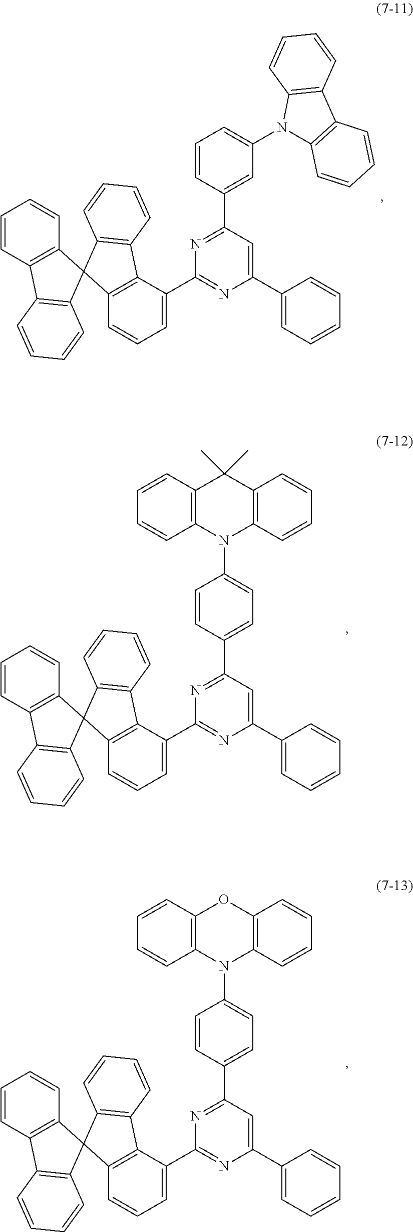 [SMILES: c1ccc(-c2cc(-c3cccc(-n4c5ccccc5c5ccccc54)c3)nc(-c3cccc4c3-c3ccccc3C43c4ccccc4-c4ccccc43)n2)cc1 ; CC1(C)c2ccccc2N(c2ccc(-c3cc(-c4ccccc4)nc(-c4cccc5c4-c4ccccc4C54c5ccccc5-c5ccccc54)n3)cc2)c2ccccc21 ; c1ccc(-c2cc(-c3ccc(N4c5ccccc5Oc5ccccc54)cc3)nc(-c3cccc4c3-c3ccccc3C43c4ccccc4-c4ccccc43)n2)cc1]

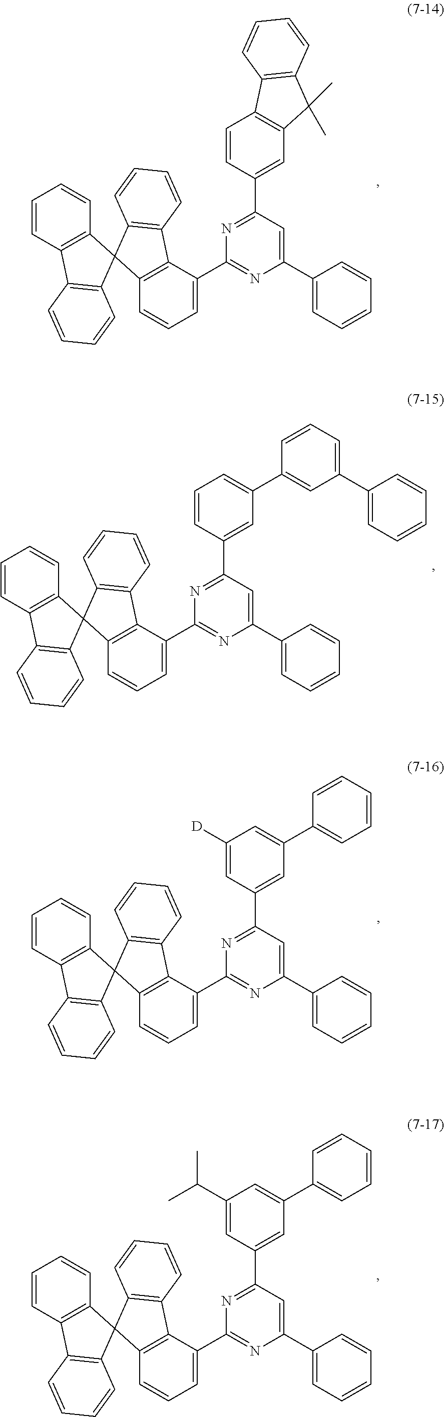 [SMILES: CC1(C)c2ccccc2-c2ccc(-c3cc(-c4ccccc4)nc(-c4cccc5c4-c4ccccc4C54c5ccccc5-c5ccccc54)n3)cc21 ; c1ccc(-c2cccc(-c3cccc(-c4cc(-c5ccccc5)nc(-c5cccc6c5-c5ccccc5C65c6ccccc6-c6ccccc65)n4)c3)c2)cc1 ; [2H]c1cc(-c2ccccc2)cc(-c2cc(-c3ccccc3)nc(-c3cccc4c3-c3ccccc3C43c4ccccc4-c4ccccc43)n2)c1 ; CC(C)c1cc(-c2ccccc2)cc(-c2cc(-c3ccccc3)nc(-c3cccc4c3-c3ccccc3C43c4ccccc4-c4ccccc43)n2)c1]

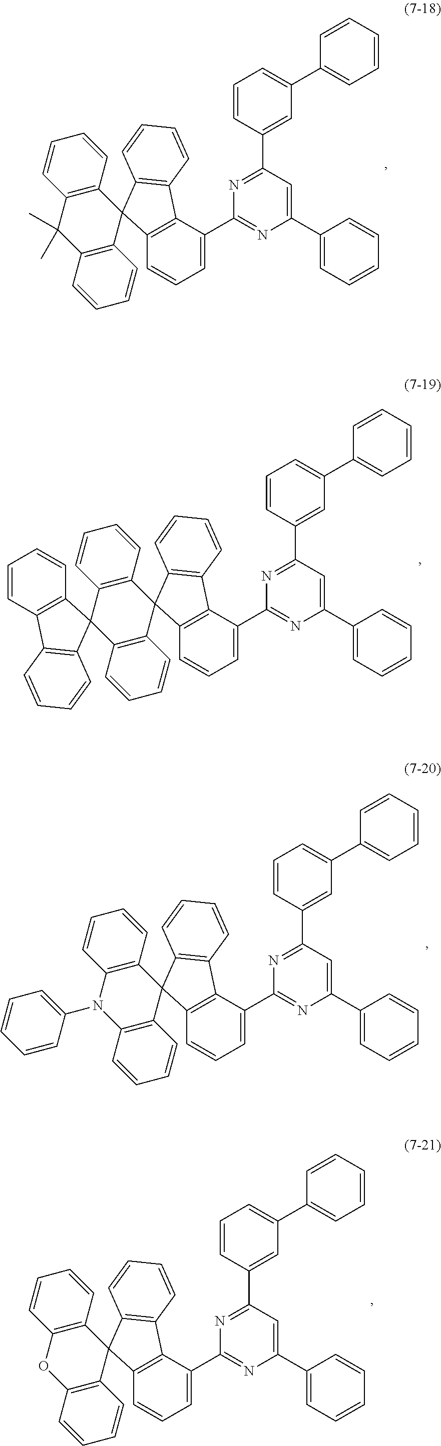 [SMILES: CC1(C)c2ccccc2C2(c3ccccc3-c3c(-c4nc(-c5ccccc5)cc(-c5cccc(-c6ccccc6)c5)n4)cccc32)c2ccccc21 ; c1ccc(-c2cccc(-c3cc(-c4ccccc4)nc(-c4cccc5c4-c4ccccc4C54c5ccccc5C5(c6ccccc6-c6ccccc65)c5ccccc54)n3)c2)cc1 ; c1ccc(-c2cccc(-c3cc(-c4ccccc4)nc(-c4cccc5c4-c4ccccc4C54c5ccccc5N(c5ccccc5)c5ccccc54)n3)c2)cc1 ; c1ccc(-c2cccc(-c3cc(-c4ccccc4)nc(-c4cccc5c4-c4ccccc4C54c5ccccc5Oc5ccccc54)n3)c2)cc1]

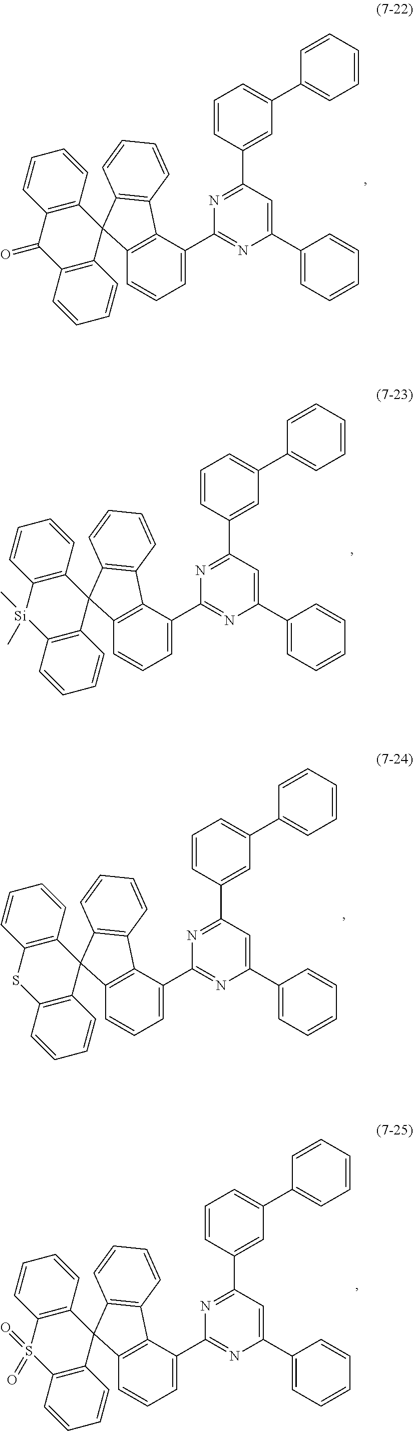 [SMILES: O=C1c2ccccc2C2(c3ccccc31)c1ccccc1-c1c(-c3nc(-c4ccccc4)cc(-c4cccc(-c5ccccc5)c4)n3)cccc12 ; C[Si]1(C)c2ccccc2C2(c3ccccc3-c3c(-c4nc(-c5ccccc5)cc(-c5cccc(-c6ccccc6)c5)n4)cccc32)c2ccccc21 ; c1ccc(-c2cccc(-c3cc(-c4ccccc4)nc(-c4cccc5c4-c4ccccc4C54c5ccccc5Sc5ccccc54)n3)c2)cc1 ; O=S1(=O)c2ccccc2C2(c3ccccc3-c3c(-c4nc(-c5ccccc5)cc(-c5cccc(-c6ccccc6)c5)n4)cccc32)c2ccccc21]

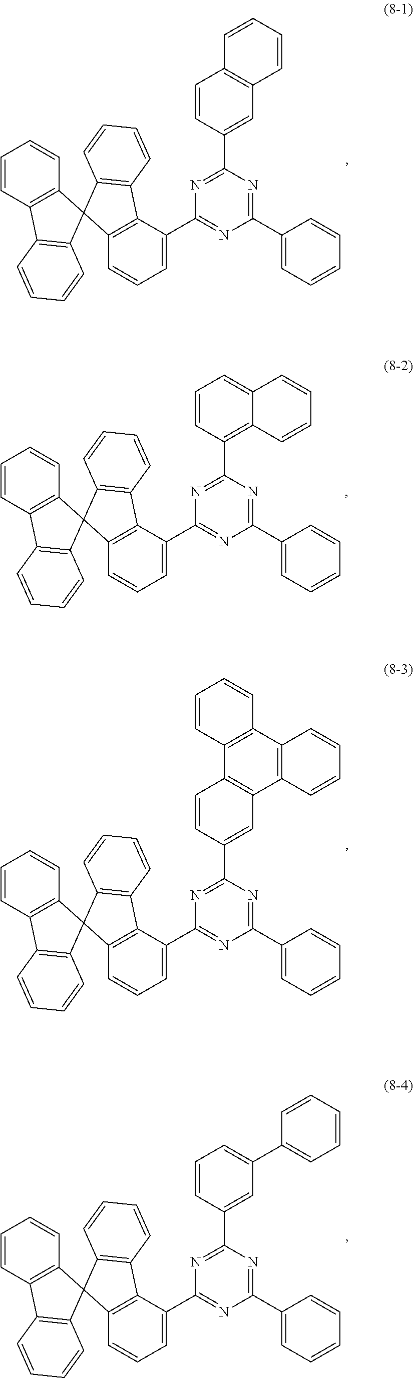 [SMILES: c1ccc(-c2nc(-c3ccc4ccccc4c3)nc(-c3cccc4c3-c3ccccc3C43c4ccccc4-c4ccccc43)n2)cc1 ; c1ccc(-c2nc(-c3cccc4c3-c3ccccc3C43c4ccccc4-c4ccccc43)nc(-c3cccc4ccccc34)n2)cc1 ; c1ccc(-c2nc(-c3ccc4c5ccccc5c5ccccc5c4c3)nc(-c3cccc4c3-c3ccccc3C43c4ccccc4-c4ccccc43)n2)cc1 ; c1ccc(-c2cccc(-c3nc(-c4ccccc4)nc(-c4cccc5c4-c4ccccc4C54c5ccccc5-c5ccccc54)n3)c2)cc1]

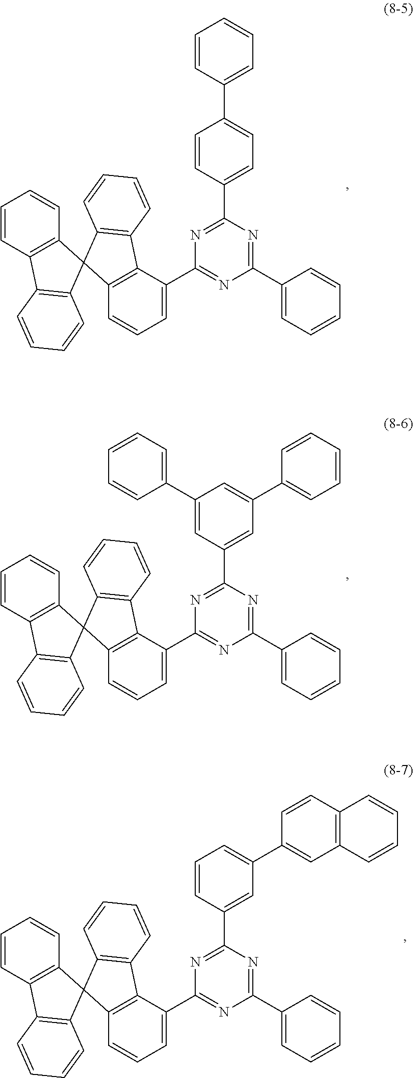 [SMILES: c1ccc(-c2ccc(-c3nc(-c4ccccc4)nc(-c4cccc5c4-c4ccccc4C54c5ccccc5-c5ccccc54)n3)cc2)cc1 ; c1ccc(-c2cc(-c3ccccc3)cc(-c3nc(-c4ccccc4)nc(-c4cccc5c4-c4ccccc4C54c5ccccc5-c5ccccc54)n3)c2)cc1 ; c1ccc(-c2nc(-c3cccc(-c4ccc5ccccc5c4)c3)nc(-c3cccc4c3-c3ccccc3C43c4ccccc4-c4ccccc43)n2)cc1]

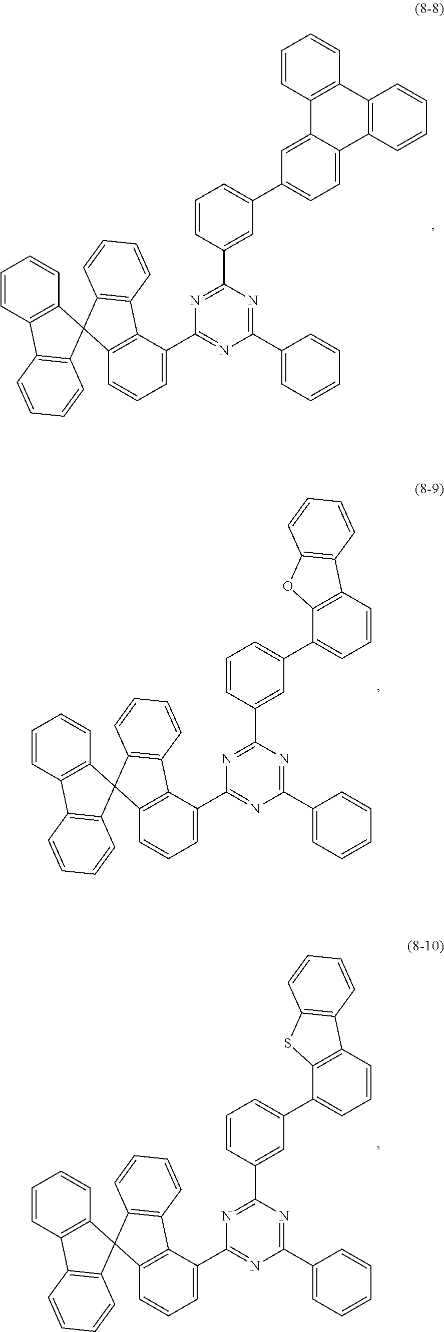 [SMILES: c1ccc(-c2nc(-c3cccc(-c4ccc5c6ccccc6c6ccccc6c5c4)c3)nc(-c3cccc4c3-c3ccccc3C43c4ccccc4-c4ccccc43)n2)cc1 ; c1ccc(-c2nc(-c3cccc(-c4cccc5c4oc4ccccc45)c3)nc(-c3cccc4c3-c3ccccc3C43c4ccccc4-c4ccccc43)n2)cc1 ; c1ccc(-c2nc(-c3cccc(-c4cccc5c4sc4ccccc45)c3)nc(-c3cccc4c3-c3ccccc3C43c4ccccc4-c4ccccc43)n2)cc1]

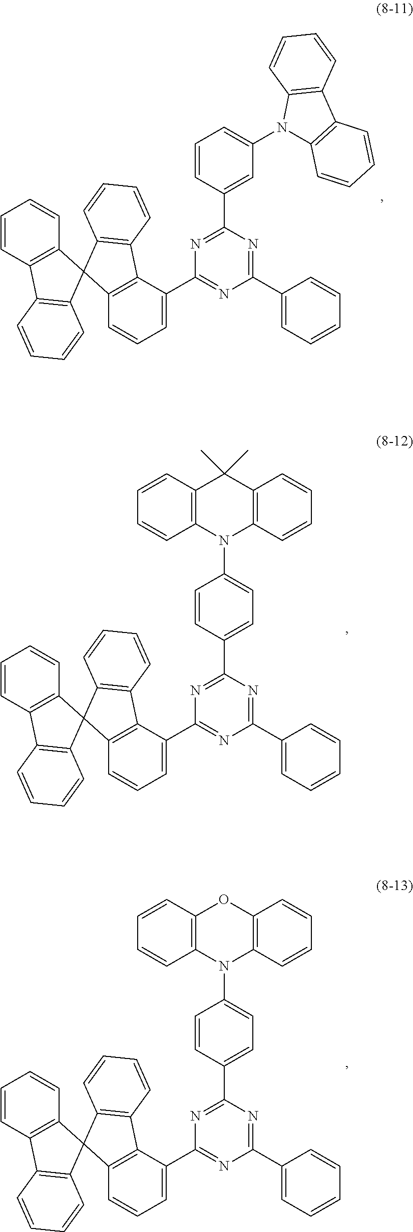 [SMILES: c1ccc(-c2nc(-c3cccc(-n4c5ccccc5c5ccccc54)c3)nc(-c3cccc4c3-c3ccccc3C43c4ccccc4-c4ccccc43)n2)cc1 ; CC1(C)c2ccccc2N(c2ccc(-c3nc(-c4ccccc4)nc(-c4cccc5c4-c4ccccc4C54c5ccccc5-c5ccccc54)n3)cc2)c2ccccc21 ; c1ccc(-c2nc(-c3ccc(N4c5ccccc5Oc5ccccc54)cc3)nc(-c3cccc4c3-c3ccccc3C43c4ccccc4-c4ccccc43)n2)cc1]

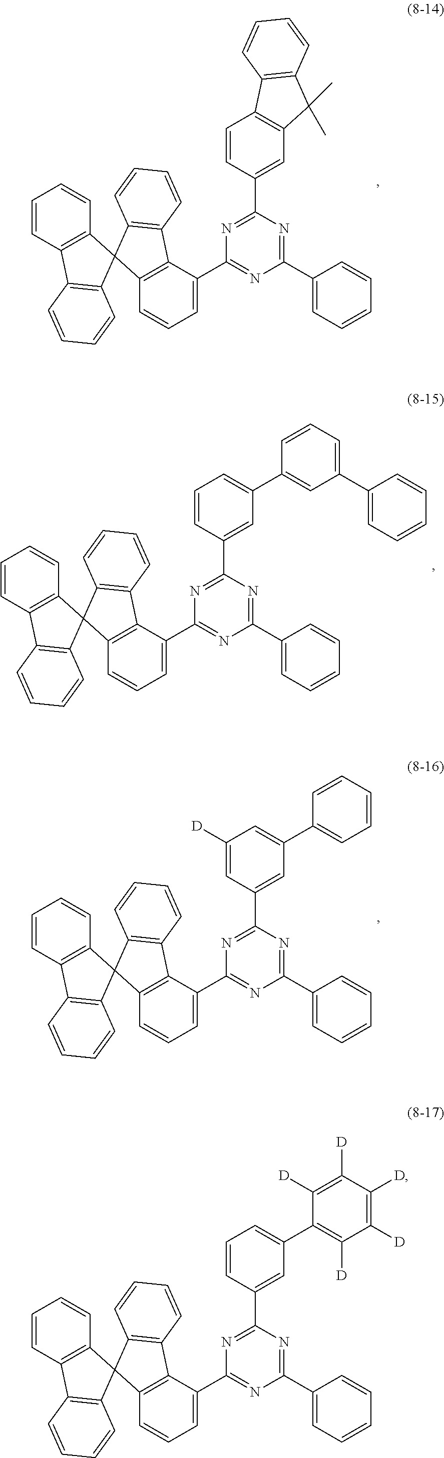

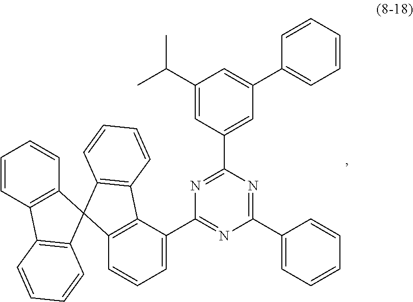 [SMILES: CC(C)c1cc(-c2ccccc2)cc(-c2nc(-c3ccccc3)nc(-c3cccc4c3-c3ccccc3C43c4ccccc4-c4ccccc43)n2)c1]

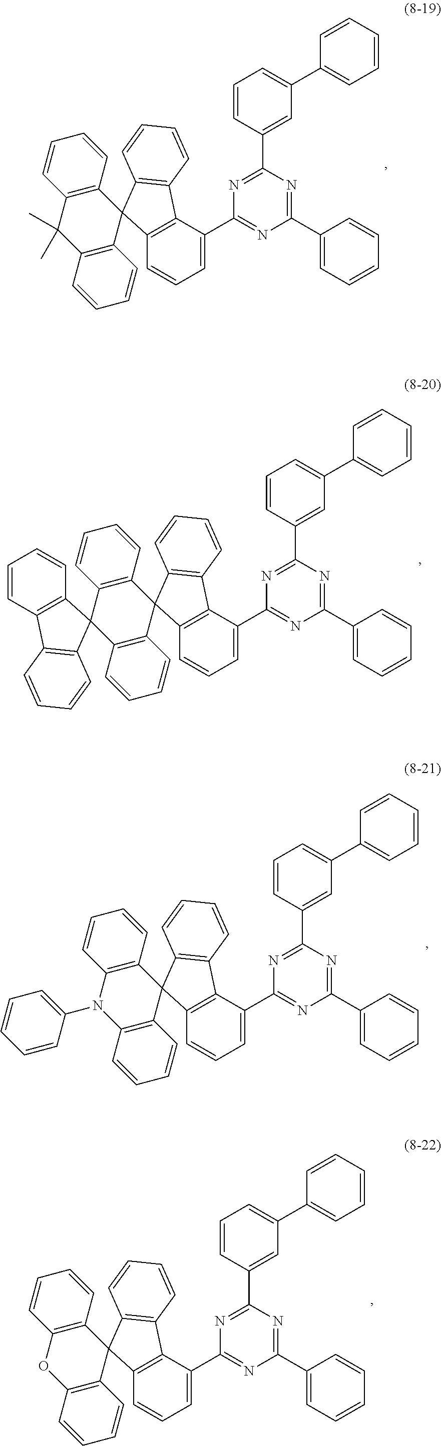 [SMILES: CC1(C)c2ccccc2C2(c3ccccc3-c3c(-c4nc(-c5ccccc5)nc(-c5cccc(-c6ccccc6)c5)n4)cccc32)c2ccccc21 ; c1ccc(-c2cccc(-c3nc(-c4ccccc4)nc(-c4cccc5c4-c4ccccc4C54c5ccccc5C5(c6ccccc6-c6ccccc65)c5ccccc54)n3)c2)cc1 ; c1ccc(-c2cccc(-c3nc(-c4ccccc4)nc(-c4cccc5c4-c4ccccc4C54c5ccccc5N(c5ccccc5)c5ccccc54)n3)c2)cc1 ; c1ccc(-c2cccc(-c3nc(-c4ccccc4)nc(-c4cccc5c4-c4ccccc4C54c5ccccc5Oc5ccccc54)n3)c2)cc1]

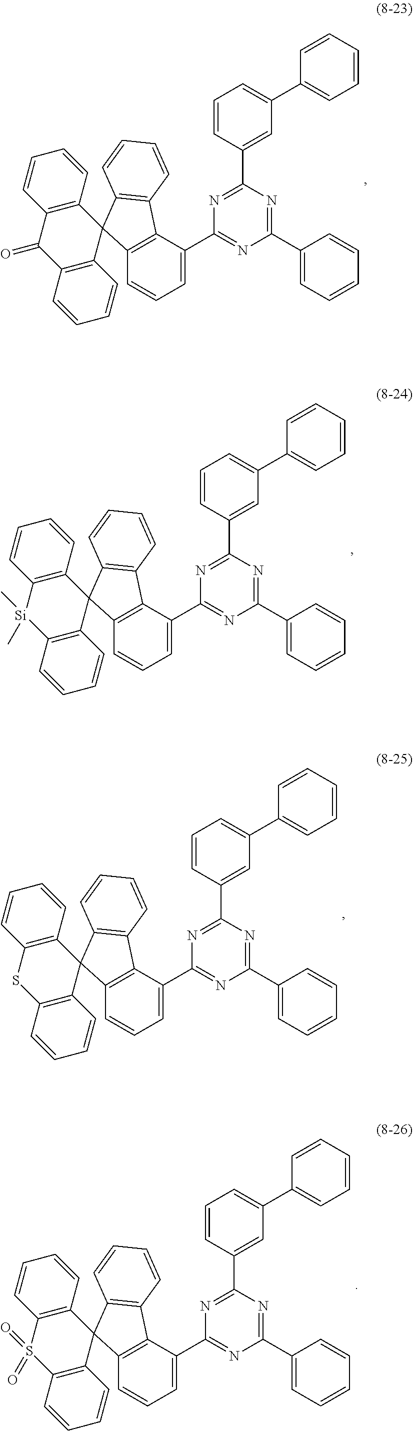 [SMILES: O=C1c2ccccc2C2(c3ccccc31)c1ccccc1-c1c(-c3nc(-c4ccccc4)nc(-c4cccc(-c5ccccc5)c4)n3)cccc12 ; C[Si]1(C)c2ccccc2C2(c3ccccc3-c3c(-c4nc(-c5ccccc5)nc(-c5cccc(-c6ccccc6)c5)n4)cccc32)c2ccccc21 ; c1ccc(-c2cccc(-c3nc(-c4ccccc4)nc(-c4cccc5c4-c4ccccc4C54c5ccccc5Sc5ccccc54)n3)c2)cc1 ; O=S1(=O)c2ccccc2C2(c3ccccc3-c3c(-c4nc(-c5ccccc5)nc(-c5cccc(-c6ccccc6)c5)n4)cccc32)c2ccccc21]

In one embodiment, the organic compound is a small molecule material. Therefore, the organic compound can be used for an evaporation OLED. In one embodiment, the organic compound has a molecular weight less than or equal to 1000 g/mol. Further, the organic compound has a molecular weight less than or equal to 900 g/mol. Still further, the organic compound has a molecular weight less than or equal to 850 g/mol. Even further, the organic compound has a molecular weight less than or equal to 800 g/mol. Even further, the organic compound has a molecular weight less than or equal to 700 g/mol.

It is noted that as used herein, the term ‘small molecule’ refers to a molecule that is not a polymer, oligomer, dendrimer, or blend. In particular, there is no repetitive structure in small molecules. The small molecule has a molecular weight less than or equal to 3000 g/mole, further, the small molecule has a molecular weight less than or equal to 2000 g/mole, and still further, the small molecule has a molecular weight less than or equal to 1500 g/mole.

In one embodiment, the organic compound has a molecular weight greater than or equal to 700 g/mole. Therefore, the organic compound can be used for a printing OLED. Further, the organic compound has a molecular weight greater than or equal to 900 g/mol. Still further, the organic compound has a molecular weight less than or equal to 1000 g/mol. Even further, the organic compound has a molecular weight less than or equal to 1100 g/mol.

In one embodiment, the organic compound has a solubility greater than or equal to 10 mg/ml in toluene at 25° C. Further, the organic compound has a solubility greater than or equal to 15 mg/ml in toluene at 25° C. Still further, the organic compound has a solubility greater than or equal to 20 mg/ml in toluene at 25° C.

The foregoing organic compounds can be used in organic functional materials. The foregoing organic compounds can be used in ink. The foregoing organic compounds can be used in organic electronic devices.

A polymer of one embodiment has at least one repeating unit comprising the foregoing organic compound. The polymer may be a conjugated polymer or a non-conjugated polymer. When the polymer is a non-conjugated high polymer, the foregoing organic compound is on the side chain of the polymer.

The foregoing polymer is used in organic functional materials. The foregoing polymer can also be used in ink. The foregoing polymer can also be used in organic electronic devices.

An organic mixture of one embodiment includes at least one organic functional material and the foregoing organic compound. In one embodiment, the organic functional material is selected from the group consisting of a hole injection material, a hole transporting material, a hole blocking material, an electron injection material, an electron transporting material, an electron blocking material, an organic host material, an organic dye or a light-emitting material. Various organic functional materials are described in detail in, for example, WO2010135519A1, US20090134784A1 and WO2011110277A1, the entire contents of which three patent documents are hereby incorporated by reference. The organic functional material may be a small molecule or a polymer material.

In one embodiment, the light-emitting material may be selected from a fluorescent light emitter, a phosphorescent light emitter, a thermally activated delayed fluorescent material or a light-emitting quantum dot.

In one embodiment, the organic functional material is selected from a phosphorescent emitter, and the organic compound is used as a host material; and based on the weight of the mixture, the organic functional material has a weight percentage of greater than 0 and less than or equal to 30 wt %. Further, the organic functional material has a weight percentage of greater than 0 and less than or equal to 25 wt %. Still further, the organic functional material has a weight percentage of greater than 0 and less than or equal to 20 wt %.

In one embodiment, the organic functional material is selected from a phosphorescent emitter and an organic host material, and the organic host material and the organic compound are used as co-host materials. And based on the weight of the mixture, the organic compound has a weight percentage of greater than 10 wt %. Further, the organic compound has a weight percentage of greater than 20 wt %. Still further, the organic compound has a weight percentage of greater than 30 wt %. Even further, the organic compound has a weight percentage of greater than 40 wt %.

In one embodiment, the organic functional material is selected from a phosphorescent emitter and an organic host material, and the organic compound is an auxiliary light-emitting material; the weight ratio of the organic compound to the phosphorescent emitter is (1:2)-(2:1). Further, the first triplet excited state of the organic compound may be higher than the first triplet excited state of the phosphorescent emitter.

In one embodiment, the organic functional material is selected from a TADF material or an ETM material.

In this embodiment, the excited state of the organic mixture will tend to occupy the lowest composite excited state, or facilitate the transfer of the energy of the triplet excited state on H1 or H2 to the exciplex state, thereby increasing the concentration of the exciplex state.

The HOMO energy level and the LUMO energy level can be measured via photoelectric effect, such as XPS (X-ray photoelectron spectroscopy) and UPS (ultraviolet photoelectron spectroscopy) or cyclic voltammetry (hereinafter referred to as CV). In addition, quantum chemical methods such as density functional theory (hereinafter referred to as DFT) can also be used to calculate the molecular orbital energy level.

The triplet energy level E_{T} of the organic material can be measured by low temperature time resolved photoluminescence spectrum or obtained by quantum simulation calculations (e.g., by Time-dependent DFT), such as by the commercial software Gaussian 03W (Gaussian Inc). Specific simulation methods can be found in WO2011141110 or as described below.

It should be noted that the absolute values of HOMO, LUMO and E_{T} are depended on the measurement method or calculation method used, and even for the same method, different HOMO/LUMO value can be given with different evaluation methods, such as with a starting point or a peak point on the CV curve. Therefore, reasonable and meaningful comparisons should be made using the same measurement method and the same evaluation method. In the embodiments of the present disclosure, the values of HOMO, LUMO and E₁ are simulations based on time-dependent DFT. However, for the application that does not affect other measurement or calculation methods, other measurement or calculation methods can also be used to obtain HOMO, LUMO and E_{T}.

The singlet emitter, the triplet emitter, and the TADF material are described in further detail below (but are not limited thereto).

### 1. Triplet Emitter

Examples of triplet host materials are not particularly limited, and any metal clathrate or organic compound may be used as a host as long as the triplet energy of it is greater than that of a light emitter, especially than that of a triplet emitter or a phosphorescent emitter. Examples of metal clathrates that may be used as triplet hosts may include, but are not limited to, the general structure as follow:

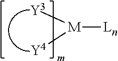

M is a metal; (Y³-Y⁴) is a bidentate ligand which is independently selected from C, N, O, P or S; L is an auxiliary ligand; m is an integer from 1 to the maximum coordination number of the metal; m+n is the maximum coordination number of the metal.

In one embodiment, the metal complex that can be used as a triplet host has the following form:

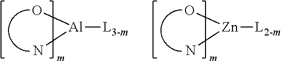

wherein (O—N) is a bidentate ligand; the metal is coordinated to the O and N atoms.

In one embodiment, M can be selected from Ir or Pt.

Examples of organic compounds that can be used as triplet hosts may be selected from compounds containing aromatic cyclic hydrocarbyl groups, such as benzene, biphenyl, triphenyl, benzo, fluorene; compounds containing heteroaromatic ring groups, such as dibenzothiophene, dibenzofuran, dibenzoselenophen, furan, thiophene, benzofuran, benzothiophene, benzoselenophene, carbazole, indolocarbazole, indolopyridine, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazin, oxadiazine, indole, benzimidazole, indoxazine, oxazole, dibenzoxazole, benzoisoxazole, benzothiazole, quinoline, isoquinoline, cinnoline, quinazoline, quinoxaline, naphthalene, phthalein, pteridine, xanthene, acridine, phenazine, phenothiazine, phenoxazine, benzofuropyridine, furopyridine, benzothienopyridine, thienopyridine, benzoselenophenopyridine and selenophenobenzodipyridine or groups comprising 2 to 10 ring structures. Among them, the groups may be the same or different types of cyclic aromatic hydrocarbyl or heteroaromatic ring groups and are linked together directly or by at least one of the following groups, such as an oxygen atom, a nitrogen atom, a sulfur atom, a silicon atom, a phosphorus atom, a boron atom, a chain structure unit and an aliphatic ring group. Among them, each Ar may be further substituted, and the substituent may be hydrogen, alkyl, alkoxy, amino, alkene, alkyne, aralkyl, heteroalkyl, aryl or heteroaryl.

In one embodiment, the triplet host material may be selected from compounds comprising at least one of the following groups:

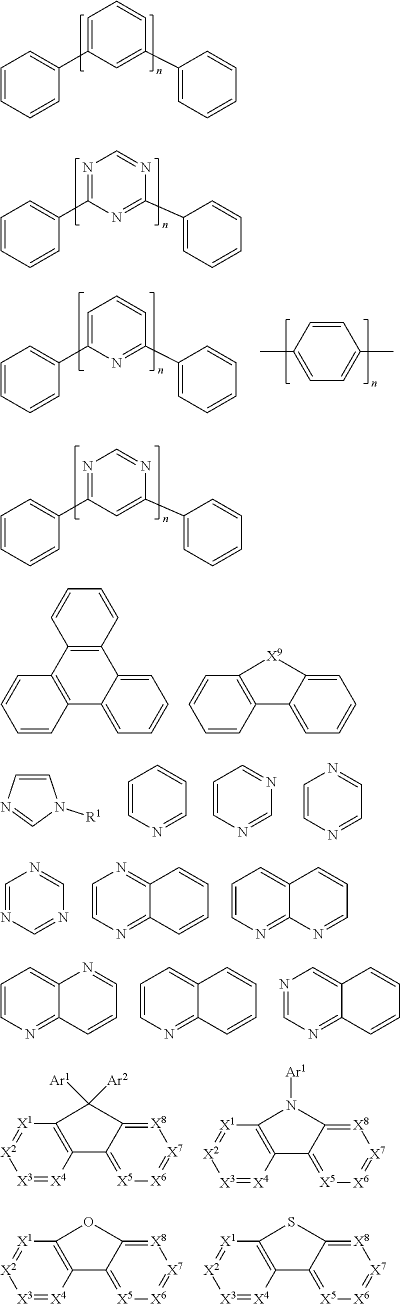

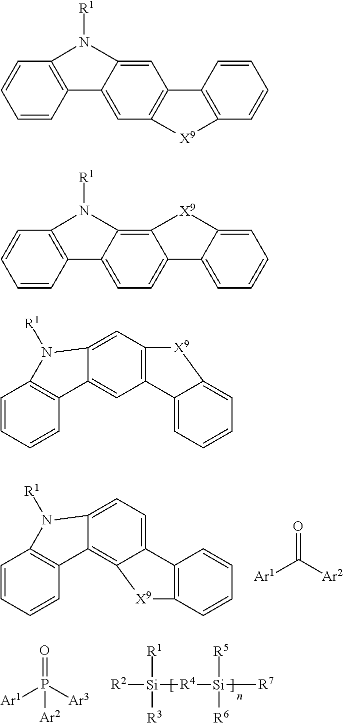

wherein R¹-R⁷ are independently selected from hydrogen, alkyl, alkoxy, amino, alkene, alkyne, aralkyl, heteroalkyl, aryl or heteroaryl; when they are aryl or heteroaryl, they have the same meaning as Ar¹ and Ar² described foregoing; n is an integer selected from 0 to 20; X¹-X⁸ are independently selected from CH or N; and X⁹ is selected from CR¹R² or NR.

Examples of suitable triplet host materials are listed in the table below.

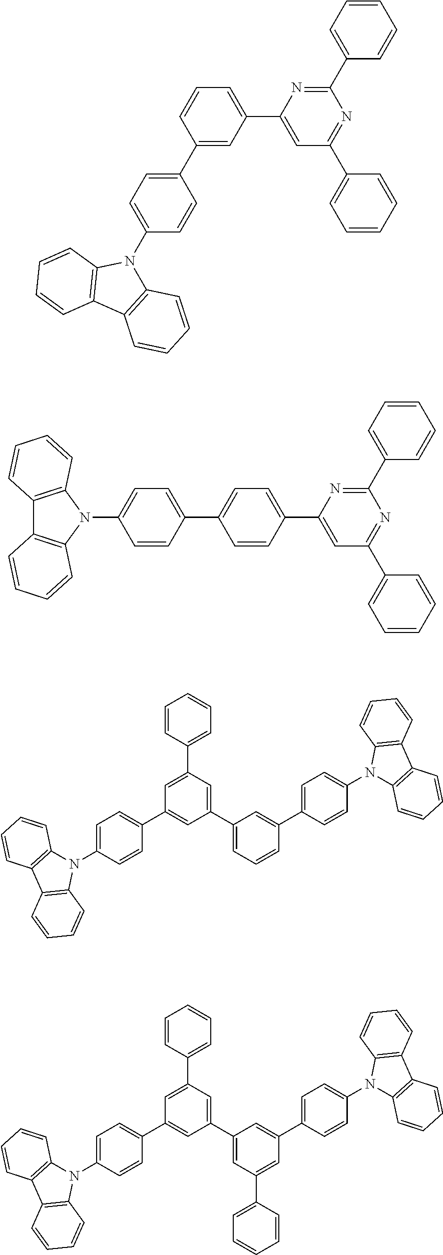 [SMILES: c1ccc(-c2cc(-c3cccc(-c4ccc(-n5c6ccccc6c6ccccc65)cc4)c3)nc(-c3ccccc3)n2)cc1 ; c1ccc(-c2cc(-c3ccc(-c4ccc(-n5c6ccccc6c6ccccc65)cc4)cc3)nc(-c3ccccc3)n2)cc1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1 ; c1ccc(-c2cc(-c3ccc(-n4c5ccccc5c5ccccc54)cc3)cc(-c3cc(-c4ccccc4)cc(-c4ccc(-n5c6ccccc6c6ccccc65)cc4)c3)c2)cc1]

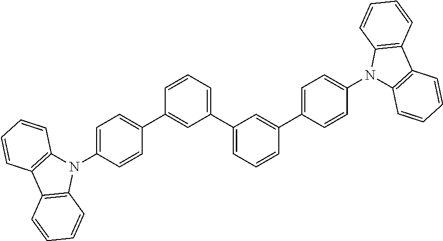 [SMILES: c1cc(-c2ccc(-n3c4ccccc4c4ccccc43)cc2)cc(-c2cccc(-c3ccc(-n4c5ccccc5c5ccccc54)cc3)c2)c1]

### 2. Phosphorescent Material

Phosphorescent materials are also called triplet emitters. The triplet emitter is a metal clathrate having the general formula M(L)n; wherein M is a metal atom; and L is an organic ligand, which may be the same or different each time when it is present, and is bonded or coordinated to the metal atom M by one or more positions. N is an integer greater than one. In one embodiment, n is selected from 1, 2, 3, 4, 5 or 6. In some embodiments, the metal clathrate is coupled to a polymer by one or more positions, especially by an organic ligand.

In one embodiment, the metal atom M is selected from a transition metal element, a lanthanide element or an actinide element. Further, the metal atom M is selected from the group consisting of Ir, Pt, Pd, Au, Rh, Ru, Os, Sm, Eu, Gd, Tb, Dy, Re, Cu or Ag. Still further, the metal atom M is selected from the group consisting of Os, Ir, Ru, Rh, Re, Pd or Pt.

In one embodiment, the triplet emitter includes a chelating ligand, i.e., a ligand, coordinated to a metal by at least two bonding sites, and particularly, the triplet emitter includes two or three identical or different bidentate or multidentate ligands. Chelating ligands help to improve stability of metal clathrates.

The organic ligand may be selected from the group consisting of a phenylpyridine derivative, a 7,8-benzoquinoline derivative, a 2(2-thienyl)pyridine derivative, a 2 (1-naphthyl)pyridine derivative, or a 2-phenylquinoline derivative. All of these organic ligands may be substituted, for example with fluorine containing groups or trifluoromethyl. The auxiliary ligand may be selected from acetone acetate or picric acid.

In one embodiment, the metal complex used as the triplet emitter has the general formula as follow:

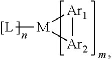

wherein M is a metal and selected from a transition metal element or a lanthanide or a lanthanide;

Ar₁ is a cyclic group which may be the same or different each time when it is present, and Ar₁ contains at least one donor atom, that is, an atom containing a lone pair of electrons, such as nitrogen or phosphorus, through which the cyclic group is connected to a metal; Ar₂ is a cyclic group which may be the same or different each time when it is present, and Ar₂ contains at least one C atom through which the cyclic group is connected to a metal; Ar₁ and Ar₂ are linked by a covalent bond with each carrying one or more substituent groups, and they may further be linked together by a substituent group; L may be the same or different each time it is present, and L is an auxiliary ligand, particularly, L is a bidentate chelating ligand, especially, L is a monoanionic bidentate chelating ligand; m is selected from 1, 2 or 3, further, m is 2 or 3, particularly further, m is 3; n is selected from 0, 1, or 2, further, n is 0 or 1, particularly further, n is 0.

Examples of some triplet emitter materials and their applications can be found in the following patent documents and literature: WO 200070655, WO 200141512, WO 200202714, WO 200215645, EP 1191613, EP 1191612, EP 1191614, WO 2005033244, WO 2005019373, US 2005/0258742, WO 2009146770, WO 2010015307, WO 2010031485, WO 2010054731, WO 2010054728, WO 2010086089, WO 2010099852, WO 2010102709, US 20070087219 A1, US 20010053462 A1, Baldo, Thompson et al. Nature 403, (2000), 750-753, US 20090061681 A1, US 20090061681 A1, J. Kido et al. Appl. Phys. Lett. 65 (1994), 2124, Kido et al. Chem. Lett. 657, 1990, US 2007/0252517 A1, Johnson et al., JACS 105, 1983, 1795, Wrighton, JACS 96, 1974, 998, Ma et al., Synth. Metals 94, 1998, 245, U.S. Pat. Nos. 6,824,895, 7,029,766, 6,835,469, 6,830,828, US 20010053462 A1, WO 2007095118 A1, US 2012004407A1, WO 2012007088A1, WO2012007087A1, WO 2012007086A1, US 2008027220A1, WO 2011157339A1, CN 102282150A, WO 2009118087A1. The entire contents of the foregoing-listed patent documents and literature are hereby incorporated by reference.

### 3. Thermally Activated Delayed Fluorescent Material (TADF):

Traditional organic fluorescent materials can only emit light using 25% singlet excitonic luminescence formed by electrical excitation, and the devices have relatively low internal quantum efficiency (up to 25%). The phosphorescent material enhances the intersystem crossing due to the strong spin-orbit coupling of the heavy atom center, the singlet exciton and the triplet exciton luminescence formed by the electric excitation can be effectively utilized, so that the internal quantum efficiency of the device can reach 100%. However, the phosphorescent materials are expensive, the material stability is poor, and the device efficiency roll-off is a serious problem, which limit its application in OLED. Thermally activated delayed fluorescent materials are the third generation of organic light-emitting materials developed after organic fluorescent materials and organic phosphorescent materials. This type of material generally has a small singlet-triplet energy level difference (ΔEₛₜ), and triplet excitons can be converted to singlet excitons by anti-intersystem crossing. This can make full use of the singlet excitons and triplet excitons formed under electric excitation. The device can achieve 100% internal quantum efficiency.

The TADF material needs to have a small singlet-triplet energy level difference, typically ΔEst<0.3 eV, further ΔEst<0.2 eV, still further ΔEst<0.1 eV, and even further ΔEst<0.05 eV. In one embodiment, TADF has good fluorescence quantum efficiency. Some TADF light-emitting materials can be found in the following patent documents: CN103483332(A), TW201309696(A), TW201309778(A), TW201350558(A), US20120217869(A1), WO2013133359(A1), WO2013154064(A1), Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc, 134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013, 3766, Adachi, et. Al. J. Mater. Chem. C., 1, 2013, 4599, Adachi, et. al. J. Phys. Chem. A., 117, 2013, 5607, the entire contents of the foregoing-listed patent or literature documents are hereby incorporated by reference.

Some examples of suitable TADF light-emitting materials are listed in the following table.

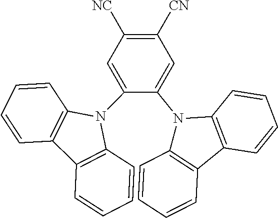 [SMILES: N#Cc1cc(-n2c3ccccc3c3ccccc32)c(-n2c3ccccc3c3ccccc32)cc1C#N]

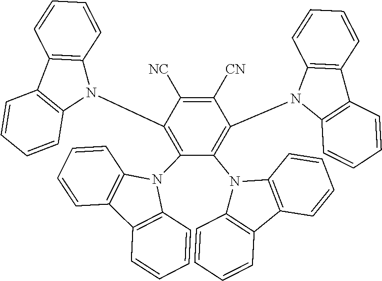 [SMILES: N#Cc1c(C#N)c(-n2c3ccccc3c3ccccc32)c(-n2c3ccccc3c3ccccc32)c(-n2c3ccccc3c3ccccc32)c1-n1c2ccccc2c2ccccc21]

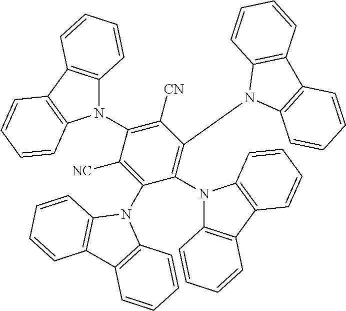 [SMILES: N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

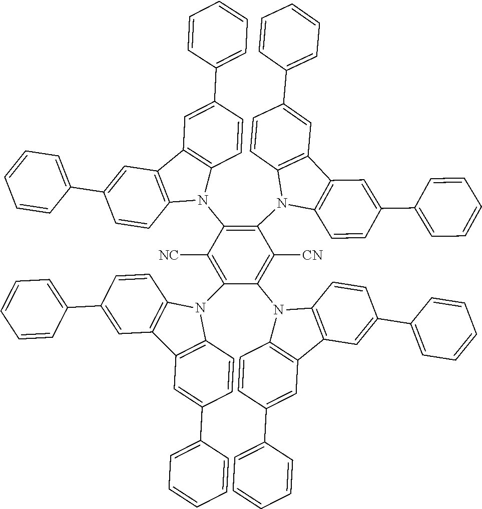 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21]

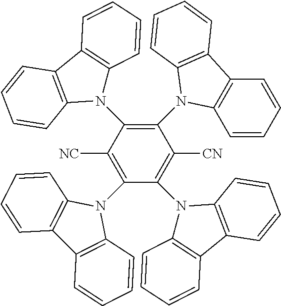 [SMILES: N#Cc1c(-n2c3ccccc3c3ccccc32)c(-n2c3ccccc3c3ccccc32)c(C#N)c(-n2c3ccccc3c3ccccc32)c1-n1c2ccccc2c2ccccc21]

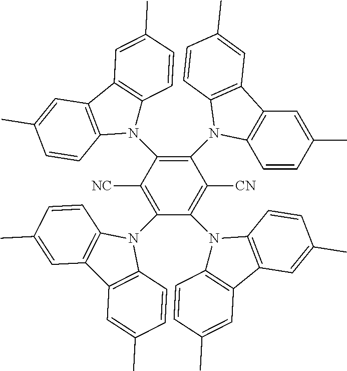

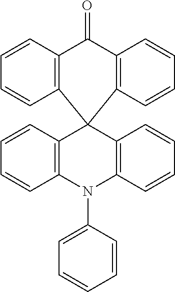 [SMILES: O=C1c2ccccc2C2(c3ccccc31)c1ccccc1N(c1ccccc1)c1ccccc12]

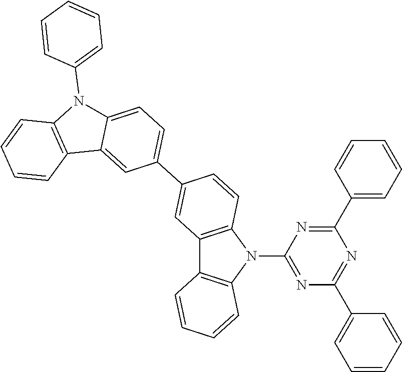 [SMILES: c1ccc(-c2nc(-c3ccccc3)nc(-n3c4ccccc4c4cc(-c5ccc6c(c5)c5ccccc5n6-c5ccccc5)ccc43)n2)cc1]

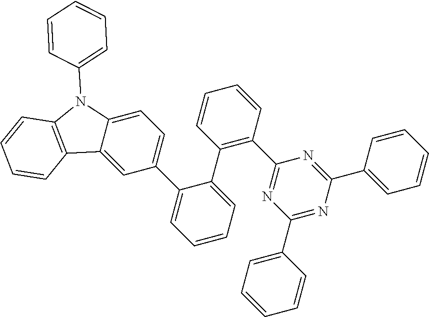 [SMILES: c1ccc(-c2nc(-c3ccccc3)nc(-c3ccccc3-c3ccccc3-c3ccc4c(c3)c3ccccc3n4-c3ccccc3)n2)cc1]

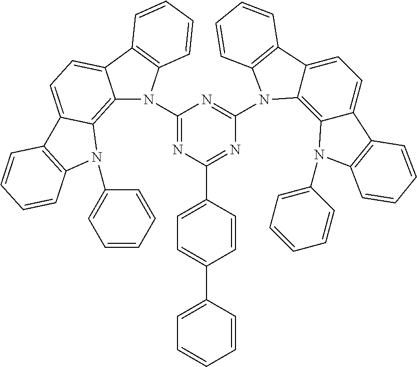 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

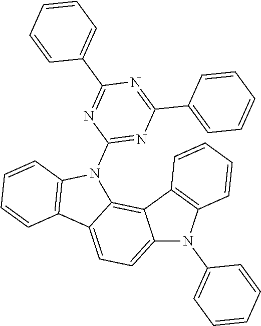 [SMILES: c1ccc(-c2nc(-c3ccccc3)nc(-n3c4ccccc4c4ccc5c(c6ccccc6n5-c5ccccc5)c43)n2)cc1]

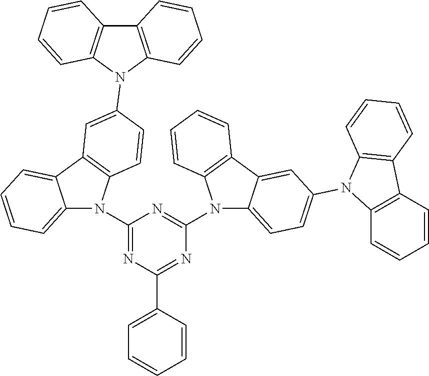 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1]

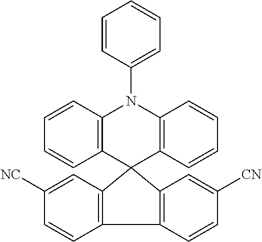 [SMILES: N#Cc1ccc2c(c1)C1(c3cc(C#N)ccc3-2)c2ccccc2N(c2ccccc2)c2ccccc21]

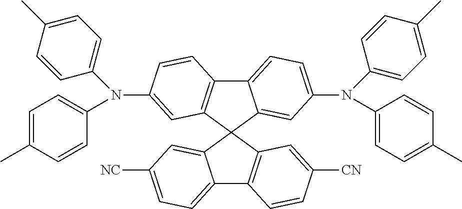

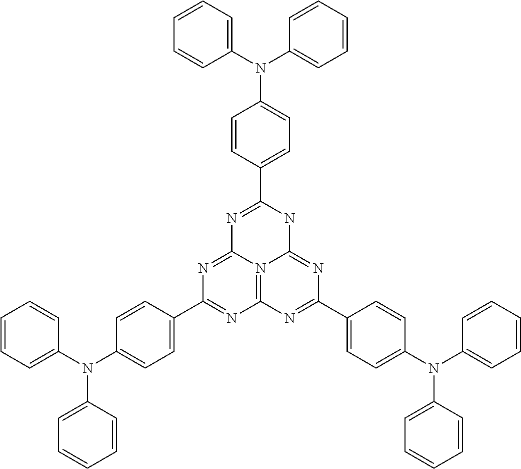

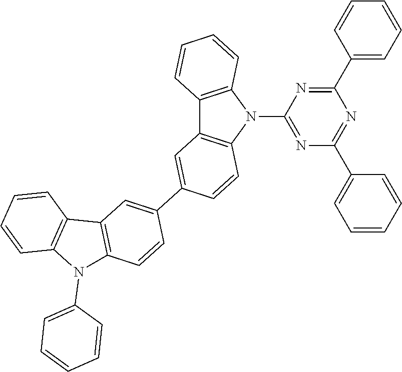 [SMILES: c1ccc(-c2nc(-c3ccccc3)nc(-n3c4ccccc4c4cc(-c5ccc6c(c5)c5ccccc5n6-c5ccccc5)ccc43)n2)cc1]

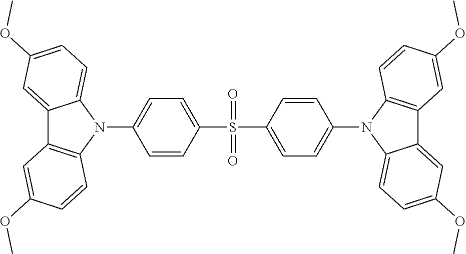 [SMILES: COc1ccc2c(c1)c1cc(OC)ccc1n2-c1ccc(S(=O)(=O)c2ccc(-n3c4ccc(OC)cc4c4cc(OC)ccc43)cc2)cc1]

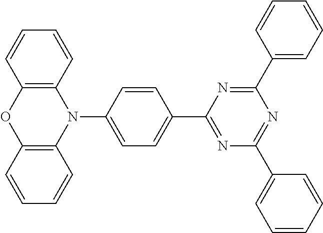 [SMILES: c1ccc(-c2nc(-c3ccccc3)nc(-c3ccc(N4c5ccccc5Oc5ccccc54)cc3)n2)cc1]

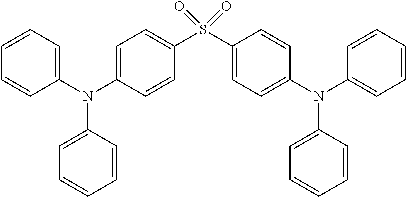 [SMILES: O=S(=O)(c1ccc(N(c2ccccc2)c2ccccc2)cc1)c1ccc(N(c2ccccc2)c2ccccc2)cc1]

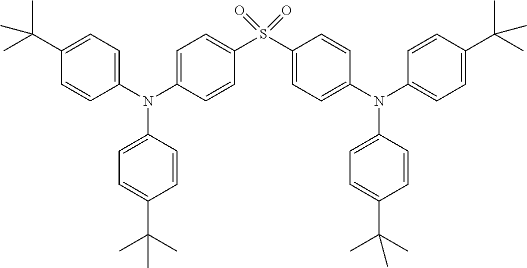 [SMILES: CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1]

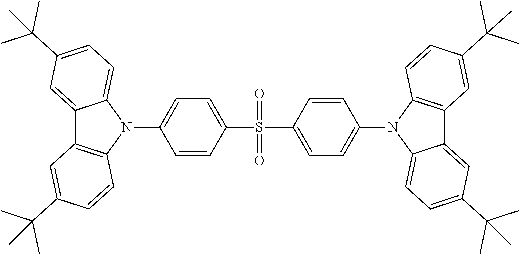 [SMILES: CC(C)(C)c1ccc2c(c1)c1cc(C(C)(C)C)ccc1n2-c1ccc(S(=O)(=O)c2ccc(-n3c4ccc(C(C)(C)C)cc4c4cc(C(C)(C)C)ccc43)cc2)cc1]

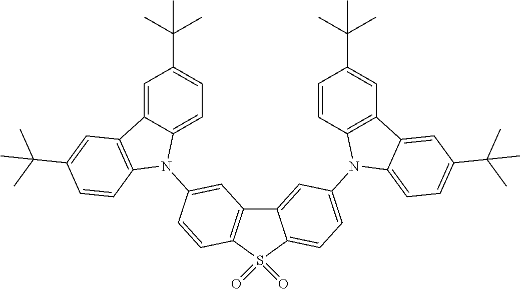 [SMILES: CC(C)(C)c1ccc2c(c1)c1cc(C(C)(C)C)ccc1n2-c1ccc2c(c1)-c1cc(-n3c4ccc(C(C)(C)C)cc4c4cc(C(C)(C)C)ccc43)ccc1S2(=O)=O]

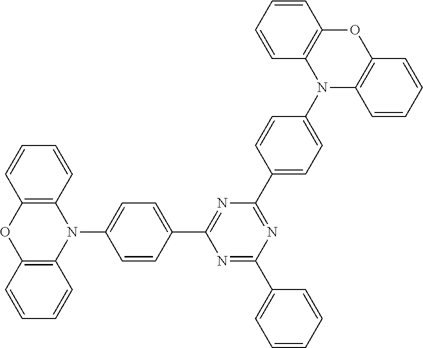 [SMILES: c1ccc(-c2nc(-c3ccc(N4c5ccccc5Oc5ccccc54)cc3)nc(-c3ccc(N4c5ccccc5Oc5ccccc54)cc3)n2)cc1]

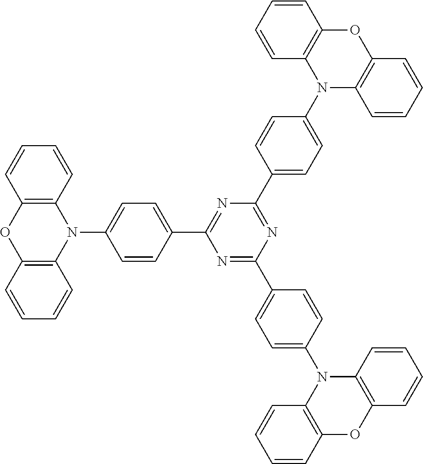 [SMILES: c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1]

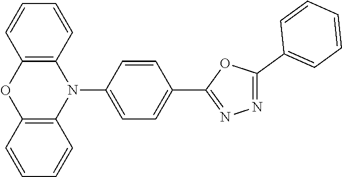 [SMILES: c1ccc(-c2nnc(-c3ccc(N4c5ccccc5Oc5ccccc54)cc3)o2)cc1]

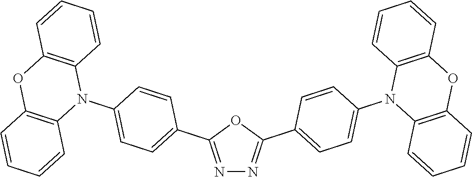 [SMILES: c1ccc2c(c1)Oc1ccccc1N2c1ccc(-c2nnc(-c3ccc(N4c5ccccc5Oc5ccccc54)cc3)o2)cc1]

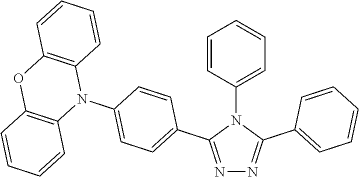 [SMILES: c1ccc(-c2nnc(-c3ccc(N4c5ccccc5Oc5ccccc54)cc3)n2-c2ccccc2)cc1]

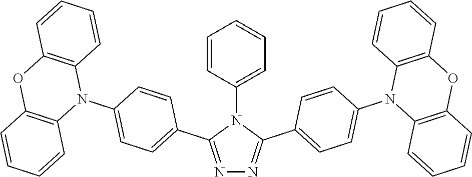 [SMILES: c1ccc(-n2c(-c3ccc(N4c5ccccc5Oc5ccccc54)cc3)nnc2-c2ccc(N3c4ccccc4Oc4ccccc43)cc2)cc1]

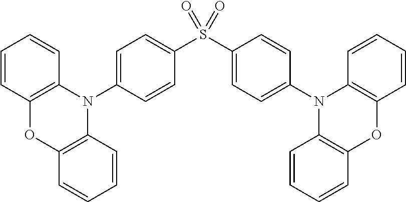 [SMILES: O=S(=O)(c1ccc(N2c3ccccc3Oc3ccccc32)cc1)c1ccc(N2c3ccccc3Oc3ccccc32)cc1]

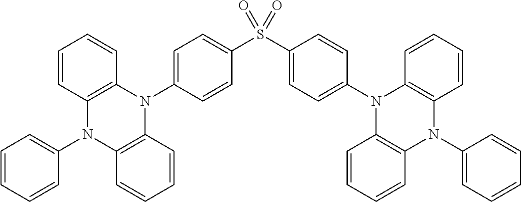 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

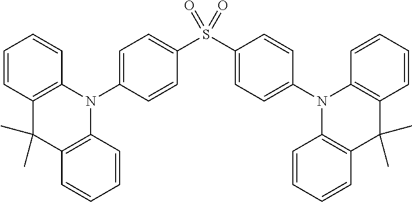

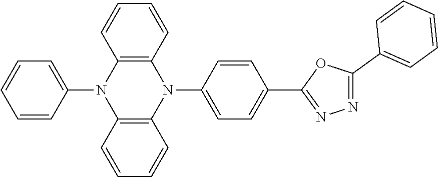 [SMILES: c1ccc(-c2nnc(-c3ccc(N4c5ccccc5N(c5ccccc5)c5ccccc54)cc3)o2)cc1]

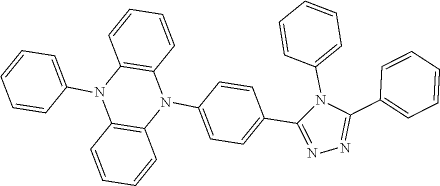 [SMILES: c1ccc(-c2nnc(-c3ccc(N4c5ccccc5N(c5ccccc5)c5ccccc54)cc3)n2-c2ccccc2)cc1]

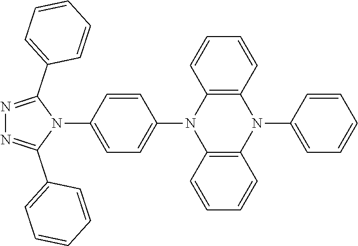 [SMILES: c1ccc(-c2nnc(-c3ccccc3)n2-c2ccc(N3c4ccccc4N(c4ccccc4)c4ccccc43)cc2)cc1]

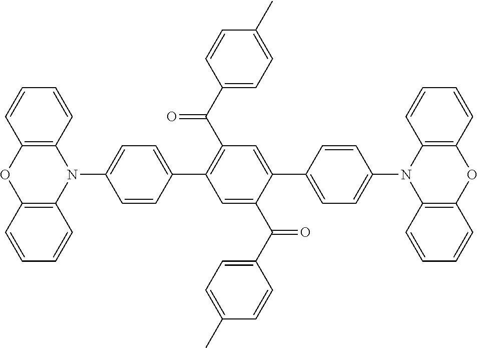 [SMILES: Cc1ccc(C(=O)c2cc(-c3ccc(N4c5ccccc5Oc5ccccc54)cc3)c(C(=O)c3ccc(C)cc3)cc2-c2ccc(N3c4ccccc4Oc4ccccc43)cc2)cc1]

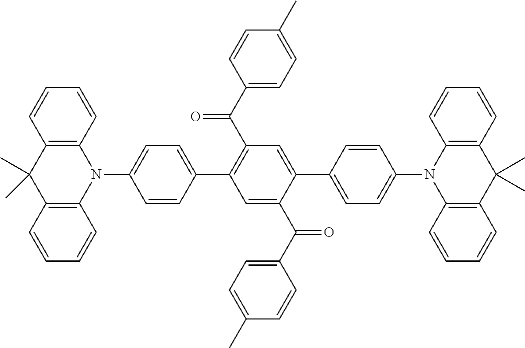

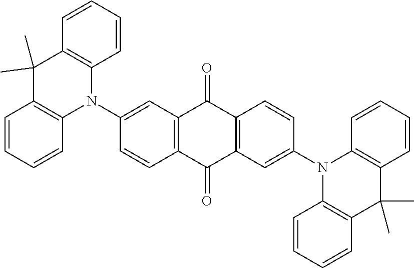

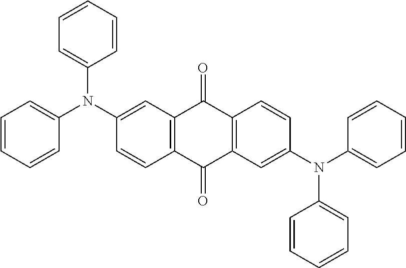 [SMILES: O=C1c2ccc(N(c3ccccc3)c3ccccc3)cc2C(=O)c2ccc(N(c3ccccc3)c3ccccc3)cc21]

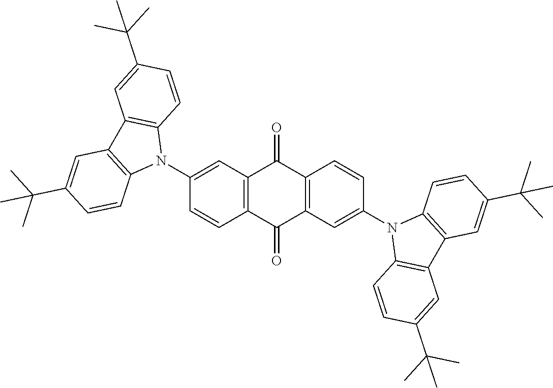 [SMILES: CC(C)(C)c1ccc2c(c1)c1cc(C(C)(C)C)ccc1n2-c1ccc2c(c1)C(=O)c1ccc(-n3c4ccc(C(C)(C)C)cc4c4cc(C(C)(C)C)ccc43)cc1C2=O]

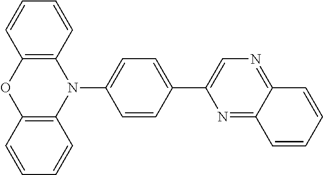 [SMILES: c1ccc2c(c1)Oc1ccccc1N2c1ccc(-c2cnc3ccccc3n2)cc1]

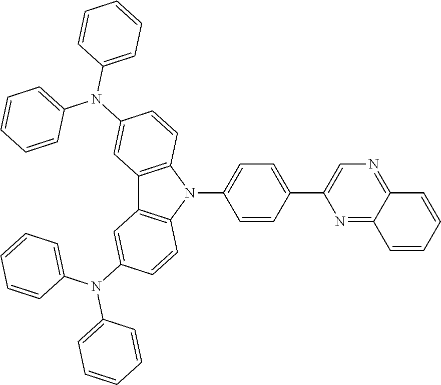 [SMILES: c1ccc(N(c2ccccc2)c2ccc3c(c2)c2cc(N(c4ccccc4)c4ccccc4)ccc2n3-c2ccc(-c3cnc4ccccc4n3)cc2)cc1]

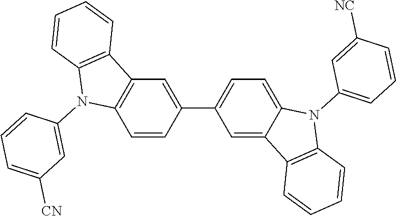 [SMILES: N#Cc1cccc(-n2c3ccccc3c3cc(-c4ccc5c(c4)c4ccccc4n5-c4cccc(C#N)c4)ccc32)c1]

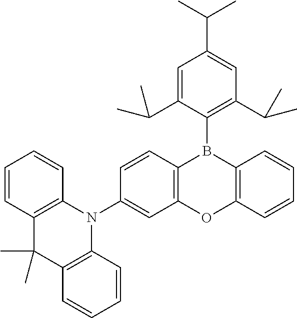 [SMILES: CC(C)c1cc(C(C)C)c(B2c3ccccc3Oc3cc(N4c5ccccc5C(C)(C)c5ccccc54)ccc32)c(C(C)C)c1]

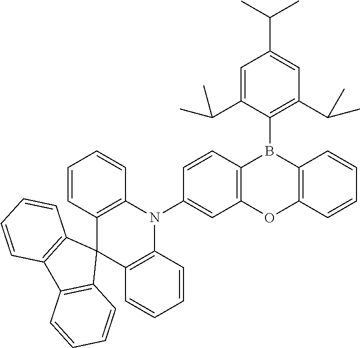 [SMILES: CC(C)c1cc(C(C)C)c(B2c3ccccc3Oc3cc(N4c5ccccc5C5(c6ccccc6-c6ccccc65)c5ccccc54)ccc32)c(C(C)C)c1]

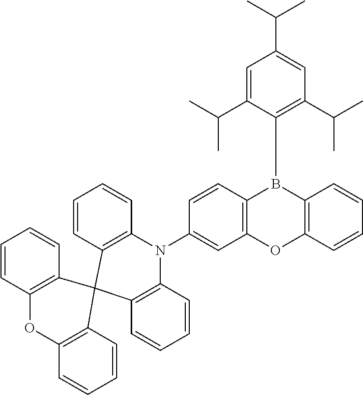 [SMILES: CC(C)c1cc(C(C)C)c(B2c3ccccc3Oc3cc(N4c5ccccc5C5(c6ccccc6Oc6ccccc65)c5ccccc54)ccc32)c(C(C)C)c1]

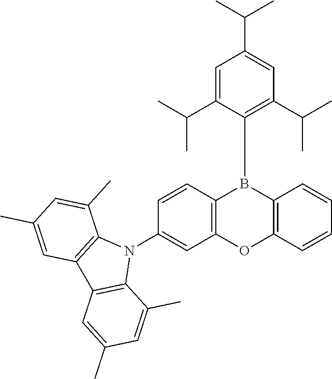 [SMILES: Cc1cc(C)c2c(c1)c1cc(C)cc(C)c1n2-c1ccc2c(c1)Oc1ccccc1B2c1c(C(C)C)cc(C(C)C)cc1C(C)C]

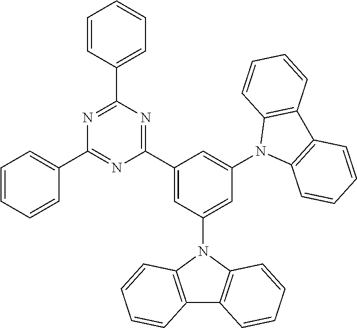 [SMILES: c1ccc(-c2nc(-c3ccccc3)nc(-c3cc(-n4c5ccccc5c5ccccc54)cc(-n4c5ccccc5c5ccccc54)c3)n2)cc1]

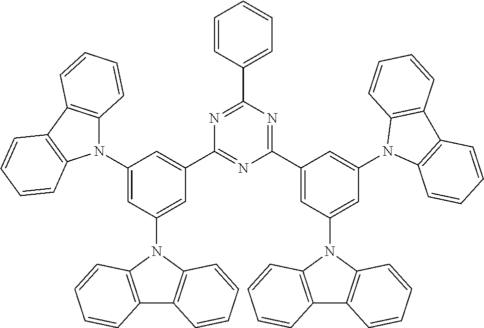 [SMILES: c1ccc(-c2nc(-c3cc(-n4c5ccccc5c5ccccc54)cc(-n4c5ccccc5c5ccccc54)c3)nc(-c3cc(-n4c5ccccc5c5ccccc54)cc(-n4c5ccccc5c5ccccc54)c3)n2)cc1]

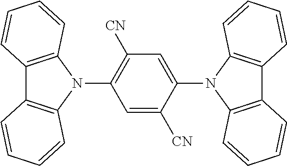 [SMILES: N#Cc1cc(-n2c3ccccc3c3ccccc32)c(C#N)cc1-n1c2ccccc2c2ccccc21]

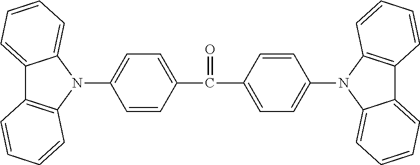 [SMILES: O=C(c1ccc(-n2c3ccccc3c3ccccc32)cc1)c1ccc(-n2c3ccccc3c3ccccc32)cc1]

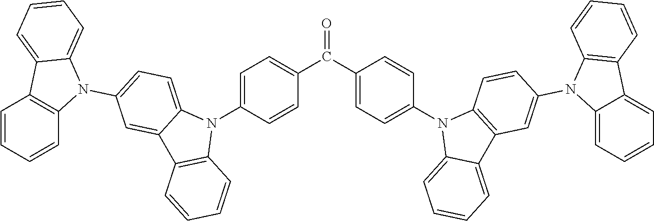 [SMILES: O=C(c1ccc(-n2c3ccccc3c3cc(-n4c5ccccc5c5ccccc54)ccc32)cc1)c1ccc(-n2c3ccccc3c3cc(-n4c5ccccc5c5ccccc54)ccc32)cc1]

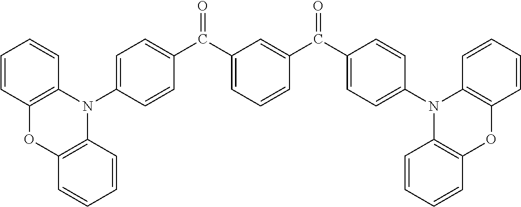 [SMILES: O=C(c1ccc(N2c3ccccc3Oc3ccccc32)cc1)c1cccc(C(=O)c2ccc(N3c4ccccc4Oc4ccccc43)cc2)c1]

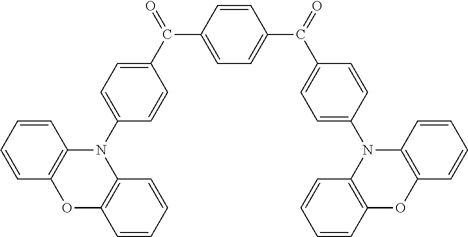 [SMILES: O=C(c1ccc(C(=O)c2ccc(N3c4ccccc4Oc4ccccc43)cc2)cc1)c1ccc(N2c3ccccc3Oc3ccccc32)cc1]

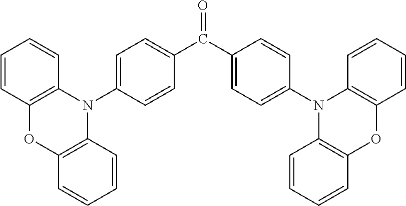 [SMILES: O=C(c1ccc(N2c3ccccc3Oc3ccccc32)cc1)c1ccc(N2c3ccccc3Oc3ccccc32)cc1]

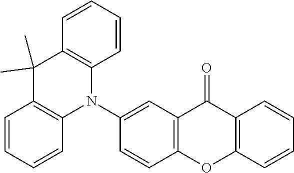 [SMILES: CC1(C)c2ccccc2N(c2ccc3oc4ccccc4c(=O)c3c2)c2ccccc21]

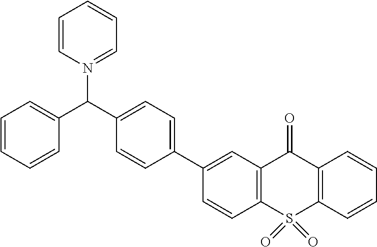

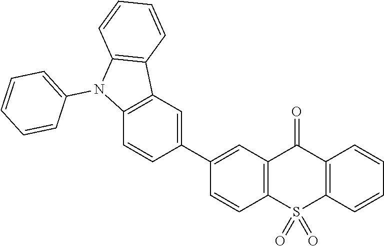 [SMILES: O=C1c2ccccc2S(=O)(=O)c2ccc(-c3ccc4c(c3)c3ccccc3n4-c3ccccc3)cc21]

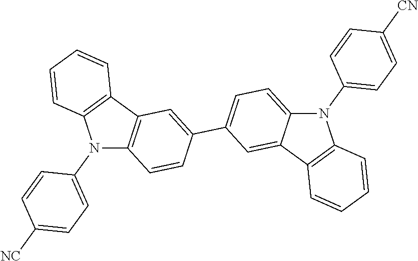 [SMILES: N#Cc1ccc(-n2c3ccccc3c3cc(-c4ccc5c(c4)c4ccccc4n5-c4ccc(C#N)cc4)ccc32)cc1]

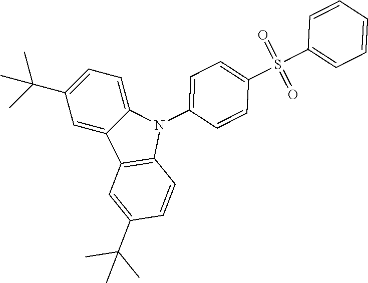 [SMILES: CC(C)(C)c1ccc2c(c1)c1cc(C(C)(C)C)ccc1n2-c1ccc(S(=O)(=O)c2ccccc2)cc1]

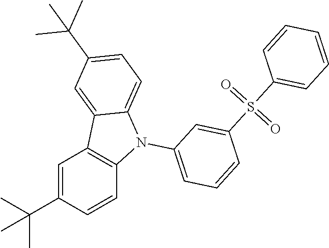 [SMILES: CC(C)(C)c1ccc2c(c1)c1cc(C(C)(C)C)ccc1n2-c1cccc(S(=O)(=O)c2ccccc2)c1]

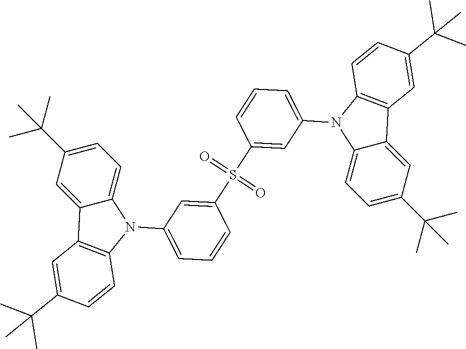 [SMILES: CC(C)(C)c1ccc2c(c1)c1cc(C(C)(C)C)ccc1n2-c1cccc(S(=O)(=O)c2cccc(-n3c4ccc(C(C)(C)C)cc4c4cc(C(C)(C)C)ccc43)c2)c1]

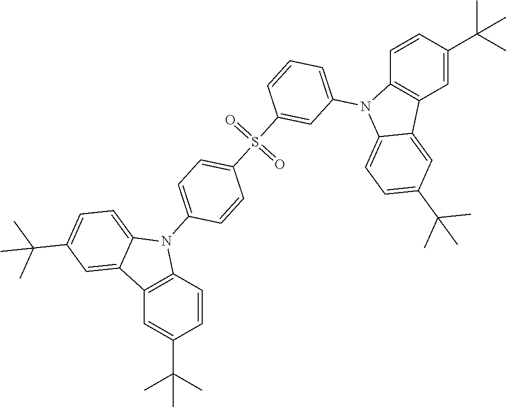 [SMILES: CC(C)(C)c1ccc2c(c1)c1cc(C(C)(C)C)ccc1n2-c1ccc(S(=O)(=O)c2cccc(-n3c4ccc(C(C)(C)C)cc4c4cc(C(C)(C)C)ccc43)c2)cc1]

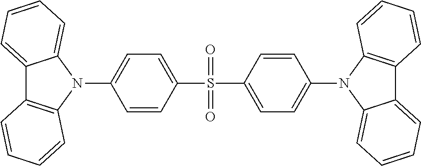 [SMILES: O=S(=O)(c1ccc(-n2c3ccccc3c3ccccc32)cc1)c1ccc(-n2c3ccccc3c3ccccc32)cc1]

The foregoing organic mixture is used in ink.

The foregoing organic mixture is used in organic electronic devices. Therefore, the lifetime of the organic electronic devices is longer.

The organic mixture of one embodiment includes at least one organic functional material and the foregoing polymer. The performance and selection of the organic functional material are as described in the foregoing embodiments, and will not be described herein.

The ink of one embodiment includes an organic solvent and the foregoing organic compound. The ink is a formulation. Therefore, the viscosity and surface tension of the ink are important parameters when the formulation is used in a printing process. Suitable surface tension parameters of the ink are suitable for a particular substrate and a particular printing method.

In one embodiment, the ink has a surface tension at an operating temperature or at 25° C. in the range of about 19 dyne/cm to 50 dyne/cm; in another embodiment, the ink has a surface tension at an operating temperature or at 25° C. in the range of 22 dyne/cm to 35 dyne/cm; and in some embodiments, the ink has a surface tension at an operating temperature or at 25° C. in the range of 25 dyne/cm to 33 dyne/cm.

In one embodiment, the ink has a viscosity at an operating temperature or 25° C. in the range of about 1 cps to 100 cps; further, the ink has a viscosity at an operating temperature or 25° C. in the range of 1 cps to 50 cps; still further, the ink has a viscosity at an operating temperature or 25° C. in the range of 1.5 cps to 20 cps; and even further, the ink has a viscosity at an operating temperature or 25° C. in the range of 4.0 cps to 20 cps. Therefore, it is more convenient for the formulation to be used in inkjet printing.

The viscosity can be adjusted by different methods, such as by selecting a suitable solvent and the concentration of the functional material in the ink. It is convenient to adjust the printing ink in an appropriate range according to the printing method used with an ink containing a metal organic complex or a polymer. Generally, the weight percentage of the organic functional material contained in the formulation is from 0.3 wt % to 30 wt %, further from 0.5 wt % to 20 wt %, still further from 0.5 wt % to 15 wt %, still further from 0.5 wt to 10 wt %, and even further from 1 wt % to 5 wt %.

In one embodiment, the organic solvent includes a first solvent selected from solvents based on aromatics or heteroaromatics. Further, the first solvent may be an aliphatic chain/ring substituted aromatic solvent, or an aromatic ketone solvent, or an aromatic ether solvent.

Examples of the first solvent are, but are not limited to, solvents based on aromatics or heteroaromatics: p-diisopropylbenzene, pentylbenzene, tetrahydronaphthalene, cyclohexyl benzene, chloronaphthalene, 1,4-dimethylnaphthalene, 3-isopropylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isopropylbiphenyl, p-cymene, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine 4-isopropylbiphenyl, α,α-dichlorodiphenylmethane, 4-(3-phenylpropyl)pyridine, benzylbenzoate, 1,1-di(3,4-dimethylphenyl)ethane, 2-isopropylnaphthalene, dibenzylether, and the like; solvents based on ketones: 1-tetralone, 2-tetralone, 2-(phenylepoxy)tetralone, 6-(methoxyl)tetralone, acetophenone, phenylacetone, benzophenone, and derivatives thereof, such as 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-decanone, 2,5-hexanedione, phorone, di-n-amyl ketone; aromatic ether solvents: 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylani sole, trans-p-propenylanisole, 1,2-dimethoxybenzene, 1-methoxynaphthalene, diphenyl ether, 2-phenoxymethyl ether, 2-phenoxytetrahydrofuran, ethyl-2-naphthyl ether, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether; and ester solvents: alkyl octoate, alkyl sebacate, alkyl stearate, alkyl benzoate, alkyl phenylacetate, alkyl cinnamate, alkyl oxalate, alkyl maleate, alkyl lactone, alkyl oleate, and the like.

Further, the first solvent can be one or more selected from aliphatic ketones, such as 2-nonanone, 3-nonanone, 5-nonanone, 2-decanone, 2,5-hexanedione, 2,6,8-trimethyl-4-nonanone, phorone, di-n-pentyl ketone, and the like; or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether.

In one embodiment, the organic solvent may also include a second solvent which is one or more selected from methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin and indene.

In one embodiment, the formulation can be a solution or a suspension. This is determined based on the compatibility between the organic mixture and the organic solvent.

In one embodiment, the organic compound in the formulation has a weight percentage from 0.01 to 20 wt %, further, the organic compound in the formulation has a weight percentage from 0.1 to 15 wt %, still further, the organic compound in the formulation has a weight percentage from 0.2 to 10 wt %, even further, the organic compound in the formulation has a weight percentage from 0.25 to 5 wt %.

In one embodiment, the foregoing formulation is used in the preparation of an organic electronic device. In particular, it is used as a coating or a printing ink in the preparation of an organic electronic device, especially, by a printing or coating preparation method.

The appropriate printing technology or coating technology includes, but is not limited to, inkjet printing, nozzle printing, typography, screen printing, dip coating, spin coating, blade coating, roller printing, twist roller printing, lithography, flexography, rotary printing, spray coating, brush coating or transfer printing or slot die coating, and the like. Particularly is gravure printing, nozzle printing and inkjet printing. The formulation may further include components which is one or more selected from a surfactant compound, a lubricant, a wetting agent, a dispersant, a hydrophobic agent, and a binder, to adjust the viscosity and the film-forming property and to improve the adhesion property. The detailed information relevant to the printing technology and requirements of the printing technology to the solution, such as solvent, concentration, and viscosity, may be referred to Handbook of Print Media: Technologies and Production Methods, Helmut Kipphan, ISBN 3-540-67326-1.

In one embodiment, the foregoing organic mixture is used in an organic electronic device. The organic electronic device may be selected from an organic light-emitting diode (OLED), an organic photovoltaic (OPV), an organic light emitting electrochemical cell (OLEEC), an organic field effect transistor (OFET), an organic light emitting field effector, an organic laser, an organic spintronic device, an organic sensor, and an organic plasmon emitting diode. In one embodiment, the organic electronic device is an OLED. Further, the organic mixture is used for a light-emitting layer for an OLED device.

The ink of another embodiment includes an organic solvent and the foregoing high polymer. The polymer is as described foregoing and will not be described herein.

The organic electronic device of one embodiment includes the foregoing organic compound. Therefore, the organic electronic device has a long lifetime.

The organic electronic device of one embodiment is an organic light-emitting diode comprising a light-emitting layer comprising the organic compound or the polymer, or the organic mixture, or the ink.

The organic electronic device of one embodiment is an organic light-emitting diode comprising an electron transporting layer comprising the organic compound or the polymer, or the organic mixture, or the ink.

In one embodiment, the organic electronic device is an electroluminescent device. The electroluminescent device can include a cathode, an anode, and a light-emitting layer therebetween, and the light-emitting layer includes the foregoing compound or organic mixture. The light-emitting layer may include a light-emitting material. The light-emitting material may be selected from a fluorescent emitter, a phosphorescent emitter and a TADF material. It should be noted that the electroluminescent device may further include a hole transporting layer which is located between the anode and the light emitting layer. The hole transporting layer includes the foregoing organic mixture. The electroluminescent device can further include a substrate on which the anode is located.

The substrate may be opaque or transparent. The transparent substrate may be used to make a transparent light-emitting device, which may be referred to Bulovic et al., Nature, 1996, 380, page 29 and Gu et al., Appl. Phys. Lett., 1996, 68, page 2606. The substrate may be rigid or elastic. The substrate may be plastic, metal, a semiconductor wafer, or glass. In some embodiments, the substrate has a smooth surface. The substrate without any surface defects is a particular ideal selection. In one embodiment, the substrate is flexible and may be selected from a polymer thin film or a plastic which has a glass transition temperature T_{g} greater than 150° C., in some embodiments, the substrate is flexible and may be selected from a polymer thin film or a plastic which has a glass transition temperature T_{g} greater than 200° C., in some embodiments, the substrate is flexible and may be selected from a polymer thin film or a plastic which has a glass transition temperature T_{g} greater than 250° C., in some embodiments, the substrate is flexible and may be selected from a polymer thin film or a plastic which has a glass transition temperature T_{g} greater than 300° C. The flexible substrate may be polyethylene terephthalate (PET) or polyethylene 2,6-naphthalate (PEN).

The anode may include a conductive metal, a metallic oxide, or a conductive polymer. The anode can inject holes easily into the hole injection layer (HIL), the hole transporting layer (HTL), or the light-emitting layer. In one embodiment, the absolute value of the difference between the work function of the anode and the HOMO energy level or the valence band energy level of the emitter in the light-emitting layer or of the p-type semiconductor material of the HIL or HTL or the electron blocking layer (EBL) is smaller than 0.5 eV, further smaller than 0.3 eV, still further smaller than 0.2 eV. Examples of the anode material include, but are not limited to, Al, Cu, Au, Ag, Mg, Fe, Co, Ni, Mn, Pd, Pt, ITO, aluminum-doped zinc oxide (AZO), and the like. The anode material can also be other materials. The anode material may be deposited by any suitable technologies, such as the suitable physical vapor deposition method which includes a radio frequency magnetron sputtering, a vacuum thermal evaporation, an electron beam, and the like. In other embodiments, the anode is patterned and structured. A patterned ITO conductive substrate may be purchased from market and used to prepare the organic electronic device according to the present embodiment.

The cathode may include a conductive metal or a metal oxide. The cathode can inject electrons easily into the EIL or ETL, or directly injected into the light-emitting layer. In one embodiment, the absolute value of the difference between the work function of the cathode and the LUMO energy level or the valence band energy level of the emitter in the light-emitting layer or of the n-type semiconductor material as the electron injection layer (EIL) or the electron transporting layer (ETL) or the hole blocking layer (HBL) is smaller than 0.5 eV, further smaller than 0.3 eV, still further smaller than 0.2 eV. All materials capable of using as the cathode of an OLED may be used as the cathode material of the organic electronic device of the present embodiment. Examples of the cathode material include, but are not limited to, Al, Au, Ag, Ca, Ba, Mg, LiF/Al, MgAg alloy, BaF₂/Al, Cu, Fe, Co, Ni, Mn, Pd, Pt, ITO, and the like. The cathode material may be deposited by any suitable technologies, such as the suitable physical vapor deposition method which includes a radio frequency magnetron sputtering, a vacuum thermal evaporation, an electron beam (e-beam), and the like.

When the electroluminescent device is an OLED, the OLED may further include other functional layers such as a hole injection layer (HIL), a hole transporting layer (HTL), an electron blocking layer (EBL), an electron injection layer (EIL), an electron transporting layer (ETL), or a hole blocking layer (HBL). Materials suitable for use in these functional layers are described in detail foregoing and in WO2010135519A1, US20090134784A1 and WO2011110277A1, the entire contents of which three patent documents are incorporated herein by reference.

In one embodiment, the electron transporting layer (ETL) or the hole blocking layer (HBL) of the electroluminescence device includes the foregoing organic compound and is prepared by a method of solution processing.

In one embodiment, the electroluminescence device has a light emission wavelength between 300 and 1000 nm, in some embodiments, the electroluminescence device has a light emission wavelength between 300 and 1000 nm between 350 and 900 nm, and in some embodiments, the electroluminescence device has a light emission wavelength between 300 and 1000 nm between 400 and 800 nm.

In one embodiment, the foregoing organic electronic device is used in an electronic equipment. The electronic equipment is selected from a display equipment, a lighting equipment, a light source, or a sensor. The organic electronic device may be an organic electroluminescent device.

The electronic device of one embodiment including the foregoing organic electronic device has a longer lifetime.

The organic electronic device of another embodiment including the foregoing polymer has a long service life and high stability. The organic electronic device is as described in the foregoing embodiment, and details are not described herein again.

The foregoing organic electronic device is used in an electronic equipment. The electronic equipment is selected from a display equipment, a lighting equipment, a light source and a sensor. Among them, the organic electronic device may be an organic electroluminescent device.

The electronic equipment of another embodiment including the foregoing organic electronic device has a longer lifetime.

### EXAMPLES

### Synthesis of Compound (4-11)

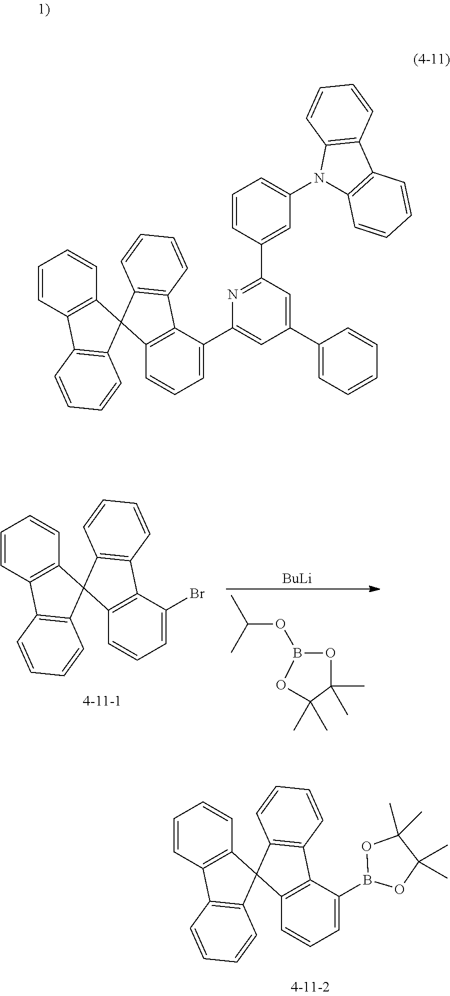

Compound 4-11-1 (31.6 g, 80 mmol) and 200 mL of anhydrous tetrahydrofuran were added to a 500 mL three-necked flask under nitrogen atmosphere. When the temperature was lowered to −78° C., 85 mmol of n-butyllithium was slowly added dropwise. After reacting for 2 hours, 90 mmol of isopropanol pinacol borate was injected once. After the reaction temperature rose to room temperature naturally and the reaction was continued for 12 hours, pure water was added to quench the reaction. Most of the solvent was rotary evaporated off, and the reaction solution was extracted with dichloromethane and washed for three times with water. The organic phase was collected, spin dried and recrystallized, with a yield rate of 80%.

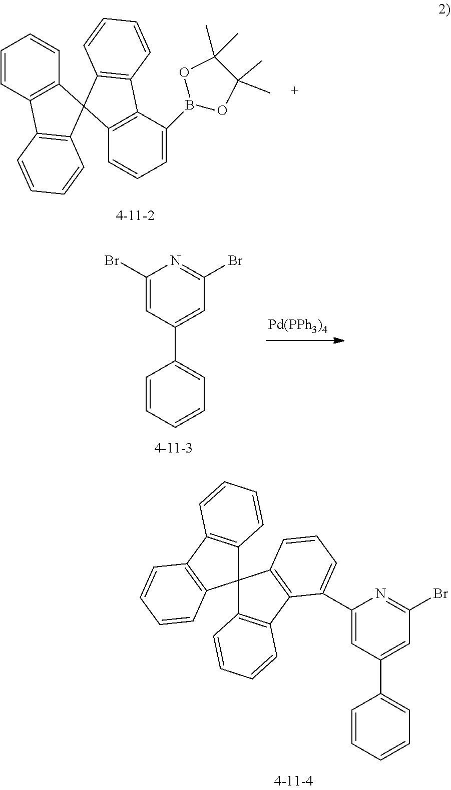 [SMILES: CC1(C)OB(c2cccc3c2-c2ccccc2C32c3ccccc3-c3ccccc32)OC1(C)C ; Brc1cc(-c2ccccc2)cc(Br)n1 ; Brc1cc(-c2ccccc2)cc(-c2cccc3c2-c2ccccc2C32c3ccccc3-c3ccccc32)n1 ; "Pd(PPh3)4"]

Compound 4-11-2 (26.5 g, 60 mmol), compound 4-11-3 (18.8 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 70%.

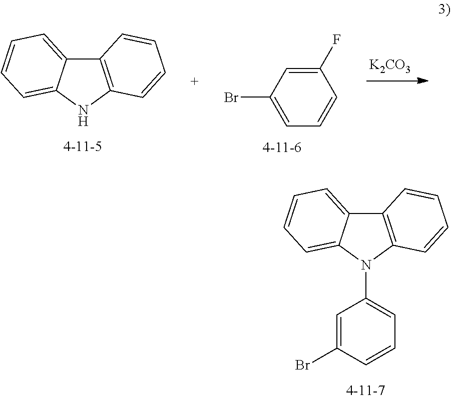

Then, compound 4-11-5 (10 g, 60 mmol), compound 4-11-6 (10.5 g, 60 mmol) and potassium carbonate (27.6 g, 200 mmol) were added to a solvent of 200 mL of N,N-dimethylformamide solvent under nitrogen atmosphere. The reaction solution was stirred and reacted at 155° C. for 12 hours. After cooled to room temperature, the reaction solution was extracted with dichloromethane and the organic solution was collected and purified by silica gel column, with a yield rate of 80%.

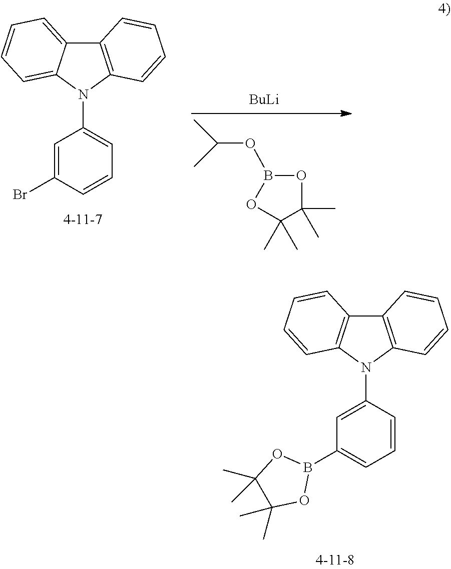

Compound 4-11-7 (12.9 g, 40 mmol) and 150 mL of anhydrous tetrahydrofuran were added to a 250 mL three-necked flask under nitrogen atmosphere. When the temperature was lowered to −78° C., 45 mmol of n-butyllithium was slowly added dropwise. After reacting for 2 hours, 50 mmol of isopropanol pinacol borate was injected once. After the reaction rose to room temperature naturally and the reaction was continued for 12 hours, pure water was added to quench the reaction. Most of the solvent was rotary evaporated off, and the reaction solution was extract with dichloromethane and washed for three times with water. The organic phase was collected, spin dried and recrystallized, with a yield rate of 90%.

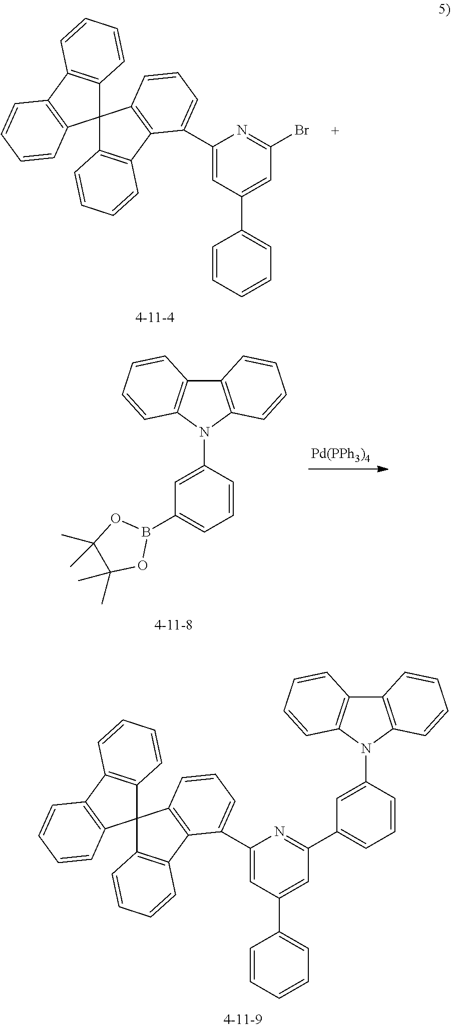 [SMILES: Brc1cc(-c2ccccc2)cc(-c2cccc3c2-c2ccccc2C32c3ccccc3-c3ccccc32)n1 ; CC1(C)OB(c2cccc(-n3c4ccccc4c4ccccc43)c2)OC1(C)C ; c1ccc(-c2cc(-c3cccc(-n4c5ccccc5c5ccccc54)c3)nc(-c3cccc4c3-c3ccccc3C43c4ccccc4-c4ccccc43)c2)cc1 ; "Pd(PPh3)4"]

Compound 4-11-4 (16.4 g, 30 mmol), compound 4-11-8 (11.1 g, 30 mmol), tetrakis(triphenylphosphine)palladium (1.23 g, 1.5 mmol), tetrabutylammonium bromide (1.3 g, 4 mmol), sodium hydroxide (1.6 g, 40 mmol), water (10 mL) and toluene (80 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 70%.

### Synthesis of Compound (6-9)

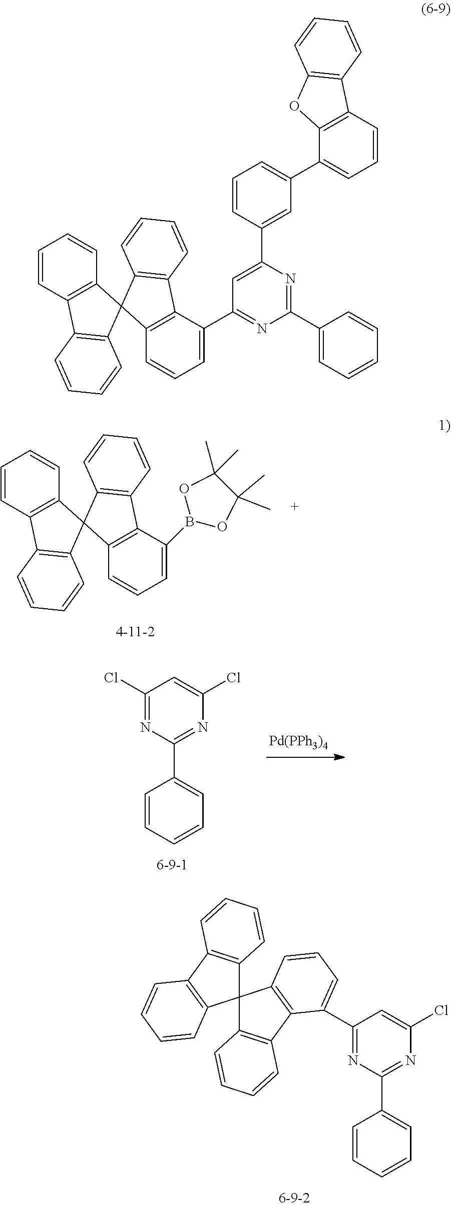

Compound 4-11-2 (26.5 g, 60 mmol), compound 6-9-1 (13.4 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 70%.

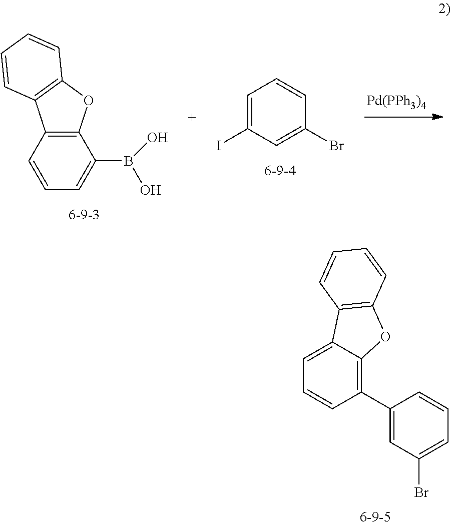 [SMILES: OB(O)c1cccc2c1oc1ccccc12 ; Brc1cccc(I)c1 ; Brc1cccc(-c2cccc3c2oc2ccccc23)c1 ; "Pd(PPh3)4"]

Compound 6-9-3 (12.7 g, 60 mmol), compound 6-9-4 (16.9 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 75%.

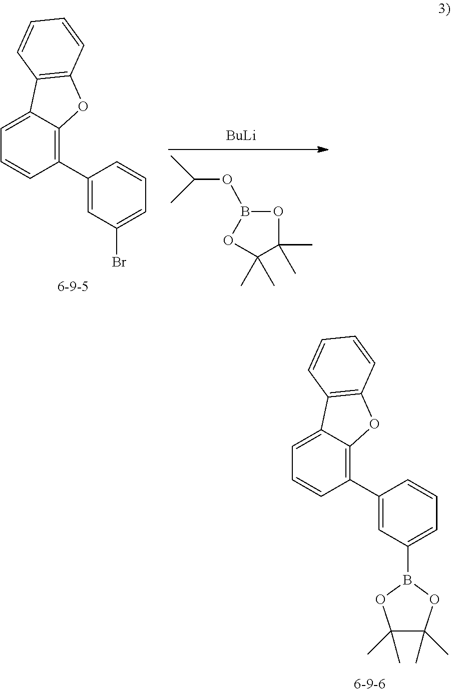

Compound 6-9-5 (12.9 g, 40 mmol) and 150 mL of anhydrous tetrahydrofuran were added to a 250 mL three-necked flask under nitrogen atmosphere. When the temperature was lowered to −78° C., 45 mmol of n-butyllithium was slowly added dropwise. After reacting for 2 hours, 50 mmol of isopropanol pinacol borate was injected once. After the reaction temperature rose to room temperature naturally and the reaction was continued for 12 hours, pure water was added to quench the reaction. Most of the solvent was rotary evaporated off, and the reaction solution was extracted with dichloromethane and washed for three times with water. The organic phase was collected, spin dried and recrystallized, with a yield rate of 90%.

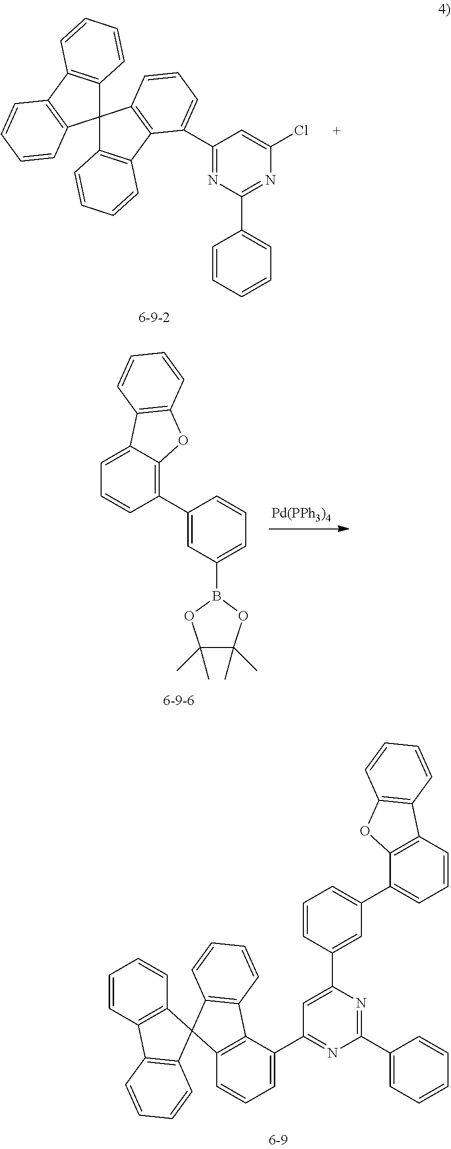 [SMILES: Clc1cc(-c2cccc3c2-c2ccccc2C32c3ccccc3-c3ccccc32)nc(-c2ccccc2)n1 ; CC1(C)OB(c2cccc(-c3cccc4c3oc3ccccc34)c2)OC1(C)C ; c1ccc(-c2nc(-c3cccc(-c4cccc5c4oc4ccccc45)c3)cc(-c3cccc4c3-c3ccccc3C43c4ccccc4-c4ccccc43)n2)cc1 ; "Pd(PPh3)4"]

Compound 6-9-2 (10.1 g, 20 mmol), compound 6-9-6 (7.4 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 800/%.

### Synthesis of Compound (8-4)

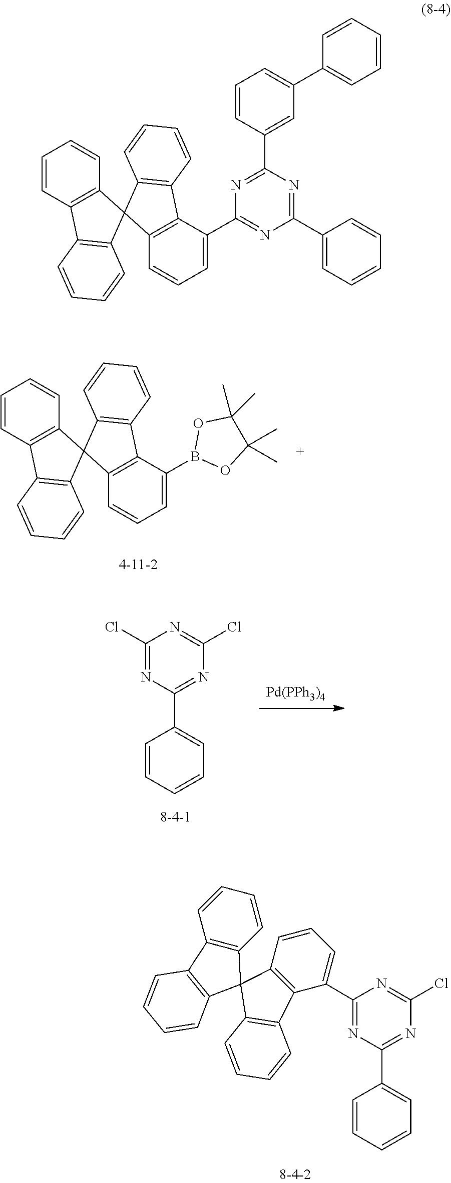 [SMILES: c1ccc(-c2cccc(-c3nc(-c4ccccc4)nc(-c4cccc5c4-c4ccccc4C54c5ccccc5-c5ccccc54)n3)c2)cc1 ; CC1(C)OB(c2cccc3c2-c2ccccc2C32c3ccccc3-c3ccccc32)OC1(C)C ; Clc1nc(Cl)nc(-c2ccccc2)n1 ; Clc1nc(-c2ccccc2)nc(-c2cccc3c2-c2ccccc2C32c3ccccc3-c3ccccc32)n1 ; "Pd(PPh3)4"]

Compound 4-11-2 (26.5 g, 60 mmol), compound 8-4-1 (13.6 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 70%.

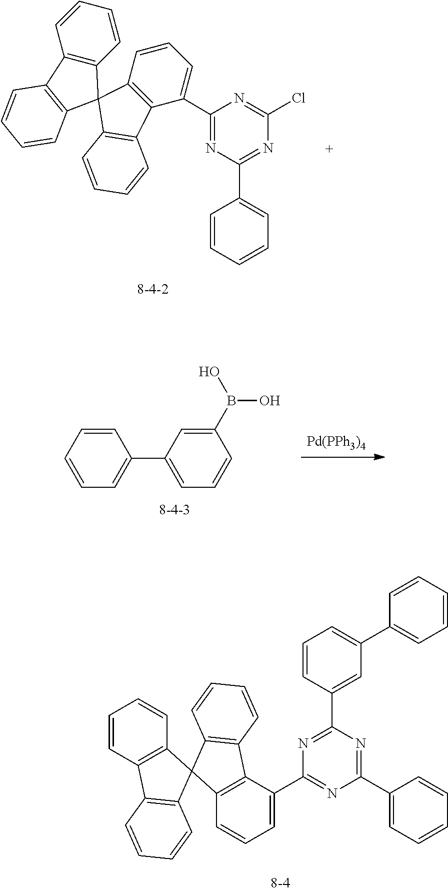 [SMILES: Clc1nc(-c2ccccc2)nc(-c2cccc3c2-c2ccccc2C32c3ccccc3-c3ccccc32)n1 ; OB(O)c1cccc(-c2ccccc2)c1 ; c1ccc(-c2cccc(-c3nc(-c4ccccc4)nc(-c4cccc5c4-c4ccccc4C54c5ccccc5-c5ccccc54)n3)c2)cc1 ; "Pd(PPh3)4"]

Compound 8-4-2 (10.1 g, 20 mmol), compound 8-4-3 (4 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 80%.

### Synthesis of Compound (8-5)

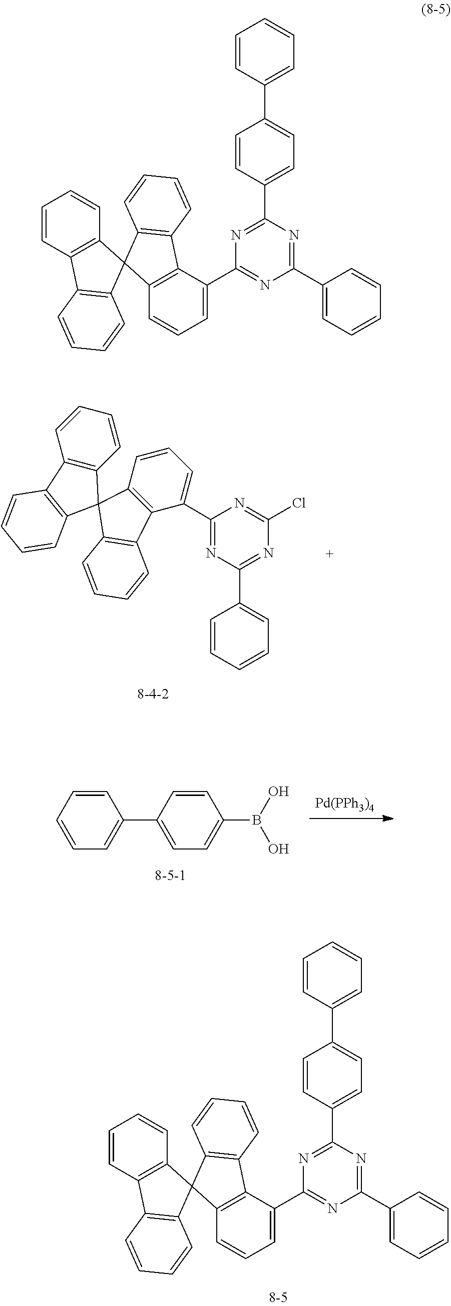 [SMILES: c1ccc(-c2ccc(-c3nc(-c4ccccc4)nc(-c4cccc5c4-c4ccccc4C54c5ccccc5-c5ccccc54)n3)cc2)cc1 ; Clc1nc(-c2ccccc2)nc(-c2cccc3c2-c2ccccc2C32c3ccccc3-c3ccccc32)n1 ; OB(O)c1ccc(-c2ccccc2)cc1 ; c1ccc(-c2ccc(-c3nc(-c4ccccc4)nc(-c4cccc5c4-c4ccccc4C54c5ccccc5-c5ccccc54)n3)cc2)cc1 ; "Pd(PPh3)4"]

Compound 8-4-2 (10.1 g, 20 mmol), compound 8-5-1 (4 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 85%.

### Synthesis of Compound (8-16)

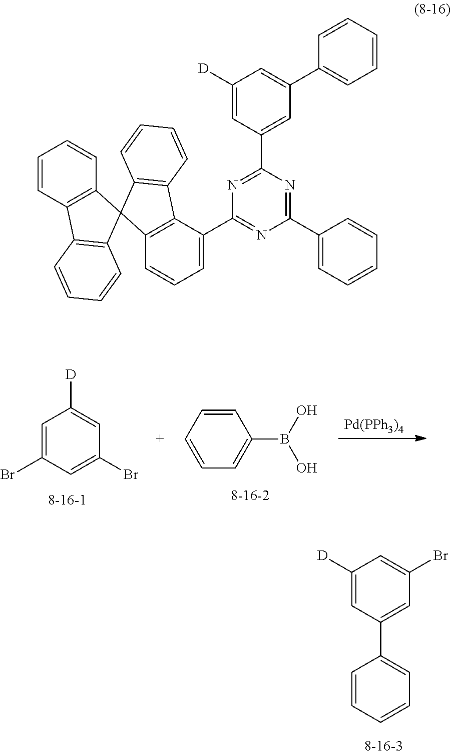 [SMILES: [2H]c1cc(-c2ccccc2)cc(-c2nc(-c3ccccc3)nc(-c3cccc4c3-c3ccccc3C43c4ccccc4-c4ccccc43)n2)c1 ; [2H]c1cc(Br)cc(Br)c1 ; OB(O)c1ccccc1 ; [2H]c1cc(Br)cc(-c2ccccc2)c1 ; "Pd(PPh3)4"]

Compound 8-16-1 (14.2 g, 60 mmol), compound 8-16-2 (7.3 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 85%.

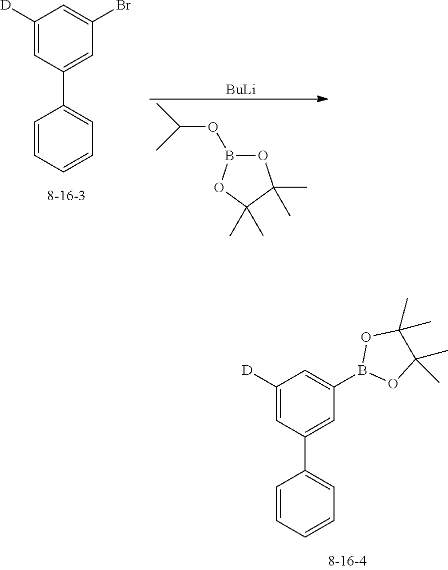

Compound 8-16-3 (9.4 g, 40 mmol) and 150 mL of anhydrous tetrahydrofuran were added to a 250 mL three-necked flask under nitrogen atmosphere. When the temperature was lowered to −78° C., 45 mmol of n-butyllithium was slowly added dropwise. After reacting for 2 hours, 50 mmol of isopropanol pinacol borate was injected once. After the reaction temperature rose to room temperature naturally and the reaction was continued for 12 hours, pure water was added to quench the reaction. Most of the solvent was rotary evaporated off, and the reaction solution was extracted with dichloromethane and washed for three times with water. The organic phase was collected, spin dried and recrystallized, with a yield rate of 90%.

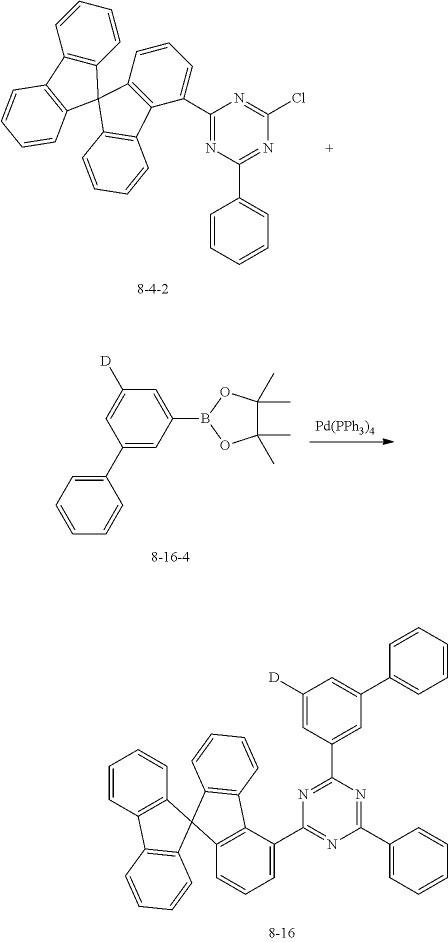 [SMILES: Clc1nc(-c2ccccc2)nc(-c2cccc3c2-c2ccccc2C32c3ccccc3-c3ccccc32)n1 ; [2H]c1cc(B2OC(C)(C)C(C)(C)O2)cc(-c2ccccc2)c1 ; [2H]c1cc(-c2ccccc2)cc(-c2nc(-c3ccccc3)nc(-c3cccc4c3-c3ccccc3C43c4ccccc4-c4ccccc43)n2)c1 ; "Pd(PPh3)4"]

Compound 8-4-2 (10.1 g, 20 mmol), compound 8-16-4 (5.6 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 80%.

### Synthesis of Compound Ref-2 of Comparative Example

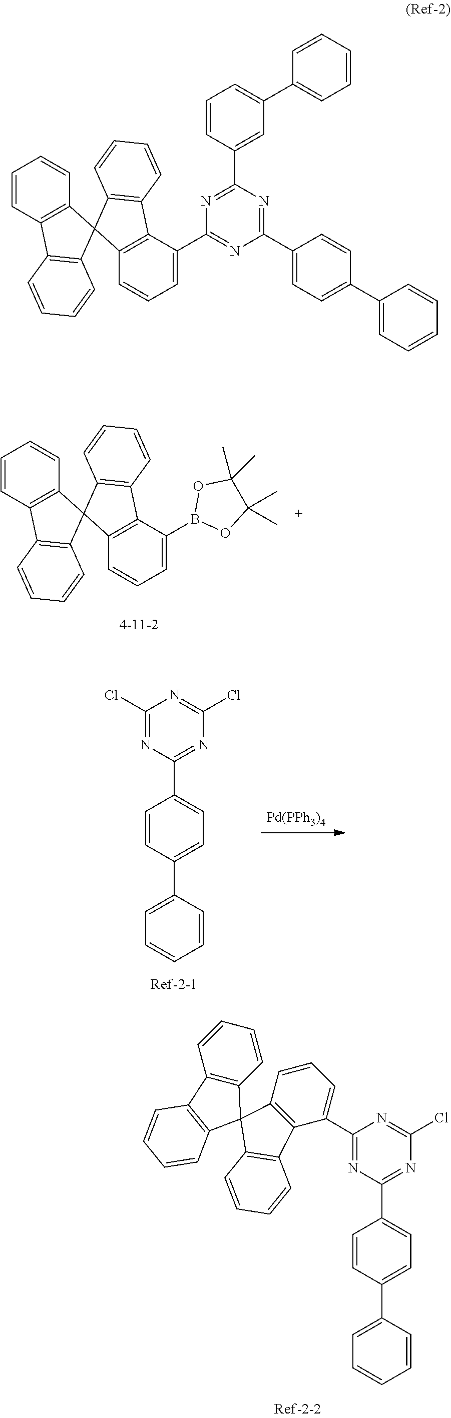 [SMILES: c1ccc(-c2ccc(-c3nc(-c4cccc(-c5ccccc5)c4)nc(-c4cccc5c4-c4ccccc4C54c5ccccc5-c5ccccc54)n3)cc2)cc1 ; CC1(C)OB(c2cccc3c2-c2ccccc2C32c3ccccc3-c3ccccc32)OC1(C)C ; Clc1nc(Cl)nc(-c2ccc(-c3ccccc3)cc2)n1 ; Clc1nc(-c2ccc(-c3ccccc3)cc2)nc(-c2cccc3c2-c2ccccc2C32c3ccccc3-c3ccccc32)n1 ; "Pd(PPh3)4"]

Compound 4-11-2 (26.5 g, 60 mmol), compound Ref-2-1 (18.1 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 75%.

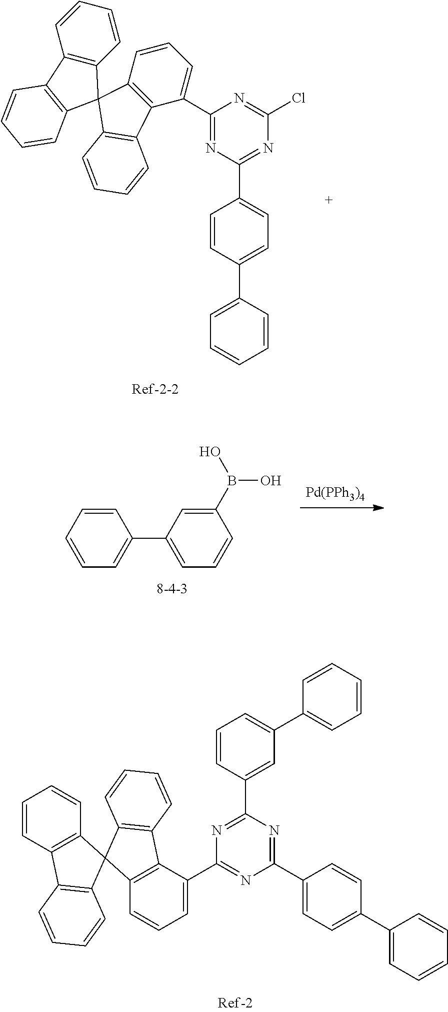 [SMILES: Clc1nc(-c2ccc(-c3ccccc3)cc2)nc(-c2cccc3c2-c2ccccc2C32c3ccccc3-c3ccccc32)n1 ; OB(O)c1cccc(-c2ccccc2)c1 ; c1ccc(-c2ccc(-c3nc(-c4cccc(-c5ccccc5)c4)nc(-c4cccc5c4-c4ccccc4C54c5ccccc5-c5ccccc54)n3)cc2)cc1 ; "Pd(PPh3)4"]

Compound Ref-2-2 (11.6 g, 20 mmol), compound 8-4-3 (4 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 85%.

### Synthesis of Compound Ref-3 of Comparative Example

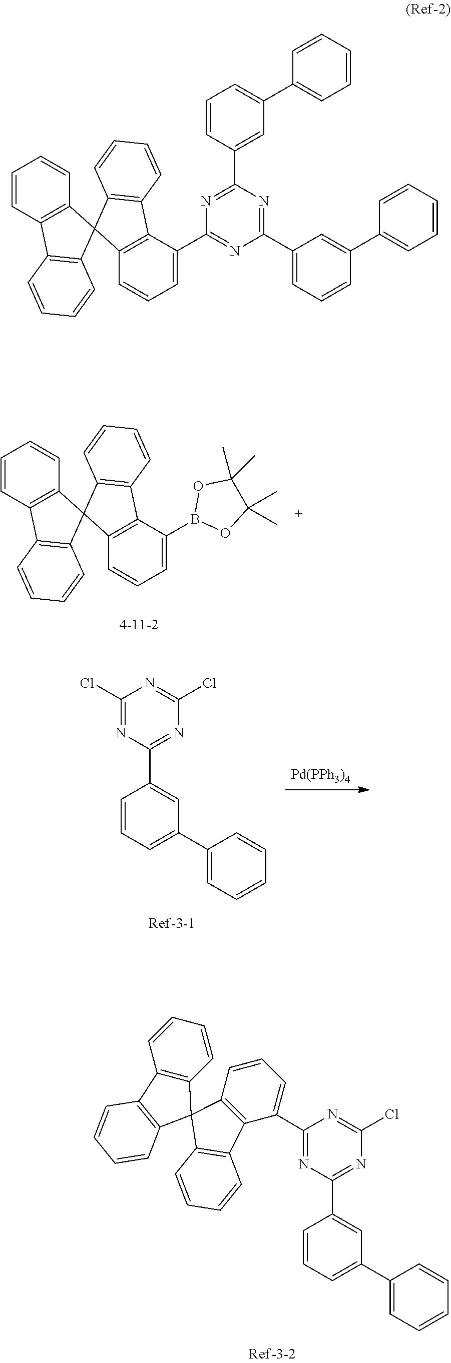 [SMILES: c1ccc(-c2cccc(-c3nc(-c4cccc(-c5ccccc5)c4)nc(-c4cccc5c4-c4ccccc4C54c5ccccc5-c5ccccc54)n3)c2)cc1 ; CC1(C)OB(c2cccc3c2-c2ccccc2C32c3ccccc3-c3ccccc32)OC1(C)C ; Clc1nc(Cl)nc(-c2cccc(-c3ccccc3)c2)n1 ; Clc1nc(-c2cccc(-c3ccccc3)c2)nc(-c2cccc3c2-c2ccccc2C32c3ccccc3-c3ccccc32)n1 ; "Pd(PPh3)4"]

Compound 4-11-2 (26.5 g, 60 mmol), compound Ref-3-1 (18.1 g, 60 mmol), tetrakis(triphenylphosphine)palladium (3.45 g, 3 mmol), tetrabutylammonium bromide (2.6 g, 8 mmol), sodium hydroxide (3.2 g, 80 mmol), water (20 mL) and toluene (150 mL) were added to a 250 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 70%.

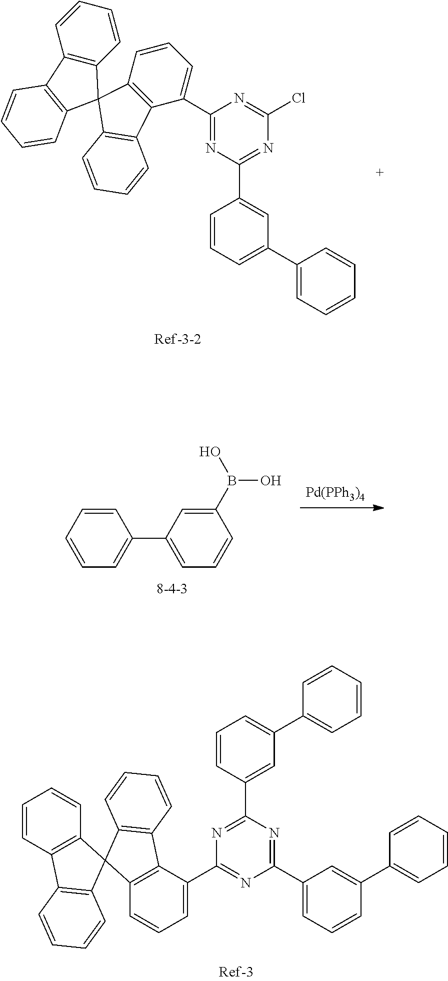 [SMILES: Clc1nc(-c2cccc(-c3ccccc3)c2)nc(-c2cccc3c2-c2ccccc2C32c3ccccc3-c3ccccc32)n1 ; OB(O)c1cccc(-c2ccccc2)c1 ; c1ccc(-c2cccc(-c3nc(-c4cccc(-c5ccccc5)c4)nc(-c4cccc5c4-c4ccccc4C54c5ccccc5-c5ccccc54)n3)c2)cc1 ; "Pd(PPh3)4"]

Compound Ref-3-2 (11.6 g, 20 mmol), compound 8-4-3 (4 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 80%.

### Synthesis of Compound Ref-4 of Comparative Example

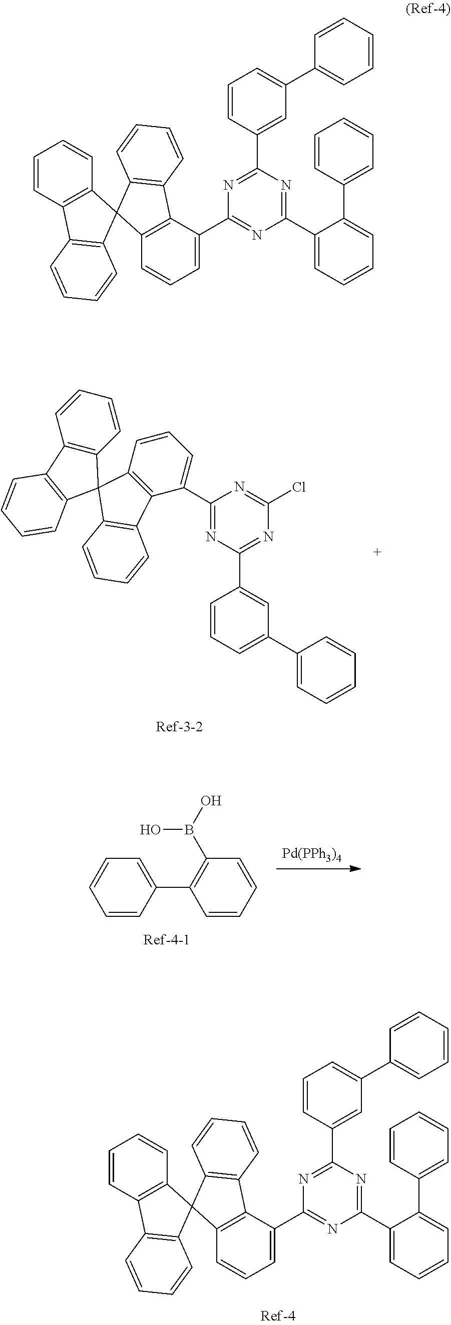 [SMILES: c1ccc(-c2cccc(-c3nc(-c4ccccc4-c4ccccc4)nc(-c4cccc5c4-c4ccccc4C54c5ccccc5-c5ccccc54)n3)c2)cc1 ; Clc1nc(-c2cccc(-c3ccccc3)c2)nc(-c2cccc3c2-c2ccccc2C32c3ccccc3-c3ccccc32)n1 ; OB(O)c1ccccc1-c1ccccc1 ; c1ccc(-c2cccc(-c3nc(-c4ccccc4-c4ccccc4)nc(-c4cccc5c4-c4ccccc4C54c5ccccc5-c5ccccc54)n3)c2)cc1 ; "Pd(PPh3)4"]

Compound Ref-3-2 (11.6 g, 20 mmol), compound Ref-4-1 (4 g, 20 mmol), tetrakis(triphenylphosphine)palladium (1.15 g, 1 mmol), tetrabutylammonium bromide (1.3 g, 4 mmol), sodium hydroxide (1.6 g, 40 mmol), water (10 mL) and toluene (60 mL) were added to a 150 mL three-necked flask and heated at 80° C. under nitrogen atmosphere. The reaction solution was stirred and reacted for 12 hours before the end of the reaction. The reaction solution was subjected to rotatory evaporation to remove most solvent and dissolved in dichloromethane, and washed for three times with water. The organic solution was collected and purified by silica gel column, with a yield rate of 70%.

### Energy Structure of the Organic Compound

The energy level of the organic material can be calculated by quantum computation, for example, using TD-DFT (time-dependent density functional theory) by Gaussian03W (Gaussian Inc.), and for the specific simulation methods, please refer to WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-density functional theory) “TD-SCF/DFT/Default Spin/B3PW91” and the basis set “6-31G (d)” (Charge 0/Spin Singlet). The HOMO and the LUMO levels are calculated using the following calibration formula, wherein S1 and T1 are used directly. Among them, HOMO represents the highest occupied orbit of the organic compound: LUMO represents the lowest unoccupied orbit of the organic compound.

HOMO(eV)=((HOMO(G)×27.212)−0.9899)/1.1206

LUMO(eV)=((LUMO(G)×27.212)−2.0041)/1.385

wherein HOMO(G) and LUMO(G) are the direct calculation results of Gaussian 03W, in units of Hartree. The results are shown in Table 1.

TABLE 1

| | | | ((LUMO + 1) − | T1 | S1 | Δ(S1 − T1) |
|---|---|---|---|---|---|---|
| Material | HOMO [eV] | LUMO [eV] | LUMO) [eV] | [eV] | [eV] | [eV] |
| HATCN | −9.04 | −5.08 | — | 2.32 | 3.17 | — |
| SFNFB | −5.26 | −2.19 | — | 2.59 | 3.22 | — |
| (4-11) | −5.75 | −2.52 | 0.20 | 2.93 | 3.30 | 0.37 |
| (6-9) | −6.03 | −2.61 | 0.18 | 2.91 | 3.14 | 0.24 |
| (8-4) | −6.01 | −2.84 | 0.19 | 2.95 | 3.26 | 0.19 |
| (8-5) | −6.01 | −2.86 | 0.20 | 2.92 | 3.29 | 0.37 |
| (8-16) | −6.01 | −2.84 | 0.19 | 2.95 | 3.26 | 0.19 |
| Ir(p-ppy)₃ | −5.17 | −2.32 | — | 2.67 | 2.90 | — |
| NaTzF₂ | −6.19 | −2.82 | — | 2.55 | 3.52 | — |

### Preparation and Characterization of OLED Devices

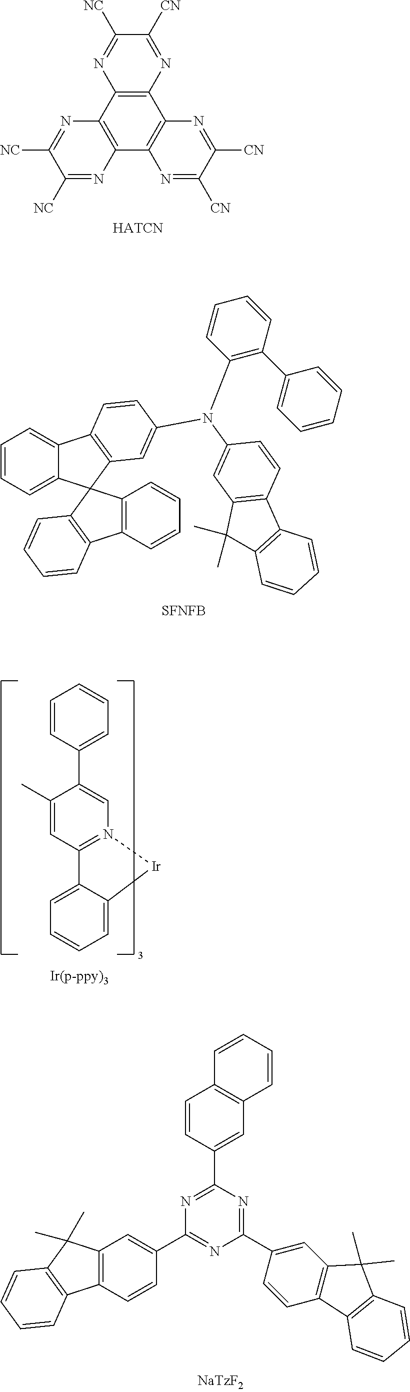

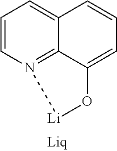

In the present embodiment, the compounds (8-4) and (8-16) were respectively used as the host materials, with Ir(p-ppy)₃ being as the light-emitting material, HATCN being as the hole injection material, SFNFB being as the hole transporting material, NaTzF₂ being as the electron transporting material, and Liq being as electron injection material in the figure above, thus constructing an electroluminescent device with a device structure of ITO/HATCN/SFNFB/host material: Ir(p-ppy)₃ (10%)/NaTzF₂: Liq/Liq/Al.

The foregoing materials HATCN, SNFFB, Ir(p-ppy)₃, NaTzF₂ and Liq are all commercially available, such as from Jilin OLED Material Tech Co., Ltd, (www.jl-oled.com), or the synthetic methods of which are all prior art, as described in the references or patents in the prior art: J. Org. Chem., 1986, 51, 5241, WO2012034627, WO2010028151, US2013248830.

The preparation process of the OLED devices described above will be described in detail below by specific examples. The structure of the OLED devices (as shown in Table 2) is ITO/HATCN/SFNFB/host material: Ir(p-ppy)₃ (10%)/NaTzF₂: Liq/Liq/Al, and the preparation steps are as follows:

a. ITO (indium tin oxide) conductive glass substrate cleaning: various solvents (such as one or more of chloroform, acetone or isopropanol) were used for cleaning, and then UV ozone treatment was applied;

b. HATCN (30 nm), SFNFB (50 nm), host material: 10% Ir(p-ppy)₃ (40 nm), NaTzF₂: Liq (30 nm), Liq (1 nm) and Al (100 nm) were formed by thermal evaporation in a high vacuum (1×10⁻⁶ mbar);

c. encapsulation: the devices were encapsulated in a nitrogen glove box with UV curable resin.

TABLE 2

| OLED device | Host material | T90@1000 nits |
|---|---|---|
| OLED1 | (8-4) | 2.6 |
| OLED2 | (8-16) | 3.1 |
| RefOLED1 | Ref-1 | 1 |
| RefOLED2 | Ref-2 | 1.15 |
| RefOLED3 | Ref-3 | 1.2 |
| RefOLED4 | Ref-4 | 0.93 |

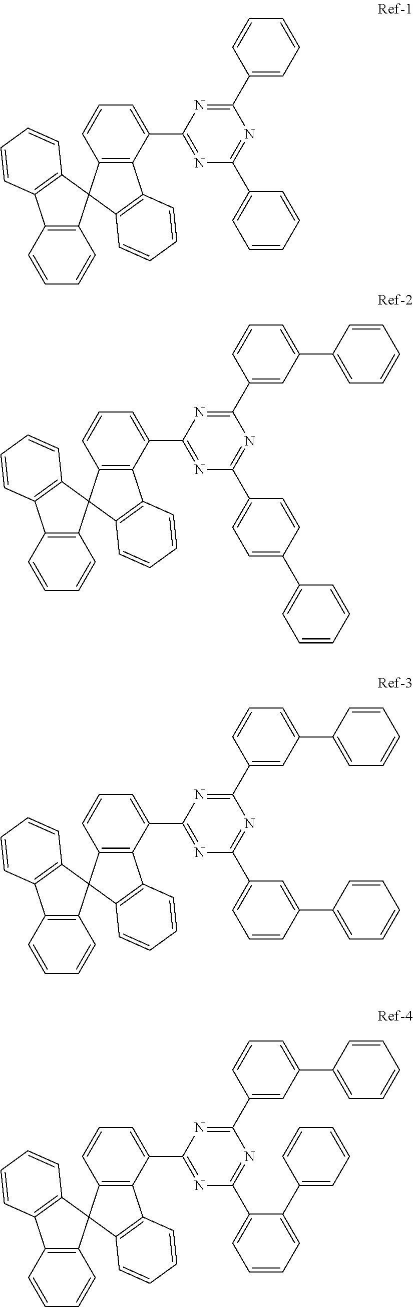 [SMILES: c1ccc(-c2nc(-c3ccccc3)nc(-c3cccc4c3-c3ccccc3C43c4ccccc4-c4ccccc43)n2)cc1 ; c1ccc(-c2ccc(-c3nc(-c4cccc(-c5ccccc5)c4)nc(-c4cccc5c4-c4ccccc4C54c5ccccc5-c5ccccc54)n3)cc2)cc1 ; c1ccc(-c2cccc(-c3nc(-c4cccc(-c5ccccc5)c4)nc(-c4cccc5c4-c4ccccc4C54c5ccccc5-c5ccccc54)n3)c2)cc1 ; c1ccc(-c2cccc(-c3nc(-c4ccccc4-c4ccccc4)nc(-c4cccc5c4-c4ccccc4C54c5ccccc5-c5ccccc54)n3)c2)cc1]

wherein for the synthesis of Ref-1, please refer to patent CN104541576A.

The current-voltage (J-V) characteristics of each OLED device are characterized by characterization equipment, while important parameters such as efficiency, lifetime and external quantum efficiency were recorded. The lifetime of each OLED device is shown in Table 2.

Among them, all the values of T90@1000 nits are relative to the value of RefOLED1. After detection, the OLED2 with deuterated host material 8-16 has the longest lifetime in the same type of devices, followed by OLED1, which are more than twice than RefOLED, RefOLED2, RefOLED3 and RefOLED4. This indicates that the simultaneous substitution with one biphenyl at position 3 and position 5 of the triazine is detrimental to the lifetime of the OLED device.

1-17. (canceled)

18. An organic compound for an organic electronic device having a general formula (1) as following: 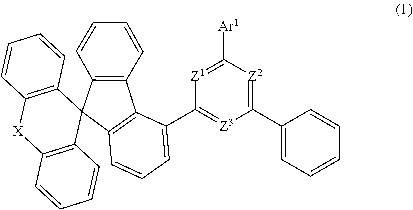
wherein
Z¹, Z², and Z³ are independently selected from N and CR′, and at least one of Z¹, Z², and Z³ is N;
X is selected from the group consisting of a single bond, N(R¹), C(R¹)₂, Si(R¹)₂, O, C═N(R¹), C═C(R¹)₂, P(R¹), P(═O)R¹, S, S═O, and SO₂;
Ar¹ is selected from an aryl group containing more than 6 ring atoms and a heteroaryl group containing more than 6 ring atoms;
R¹ is selected from the group consisting of H, D, F, CN, carbonyl, sulfonyl, alkoxy, an alkyl group containing 1 to 30 carbon atoms, a cycloalkyl group containing 3 to 30 carbon atoms, and an aryl group containing 5 to 60 ring atoms or a heteroaryl group containing 5 to 60 ring atoms.

19. The organic compound according to claim 18, wherein X is selected from the group consisting of a single bond, N(R¹), C(R¹)₂, O, and S; R¹ is selected from the group consisting of H, D, F, CN, carbonyl, sulfonyl, alkoxy, an alkyl group containing 1 to 30 carbon atoms, a cycloalkyl group containing 3 to 30 carbon atoms, and an aryl group containing 5 to 60 ring atoms or a heteroaryl group containing 5 to 60 ring atoms.

20. The organic compound according to claim 18, wherein Z¹, Z², and Z³ are independently selected from N and CR′, and one or two of Z¹, Z², and Z³ is N.

21. The organic compound according to claim 18, wherein the Ar¹ is one selected from the group consisting of: 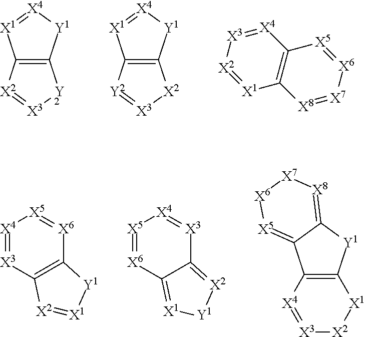
wherein
X¹, X², X³, X⁴, X⁵, X⁶, X⁷ and X⁸ are independently selected from CR² and N;
Y¹ and Y² are independently selected from CR²R³, SiR²R³, NR², C(═O), S, and O;
R² and R³ are one or more independently selected from H, D, a linear alkyl group containing 1 to 20 C atoms, a linear alkoxy group containing 1 to 20 C atoms, a linear thioalkoxy group containing 1 to 20 C atoms, a branched alkyl group containing 3 to 20 C atoms or a cyclic alkyl group containing 3 to 20 C atoms, a branched alkoxy group containing 3 to 20 C atoms or a cyclic alkoxy group containing 3 to 20 C atoms, a branched thioalkoxy group containing 3 to 20 C atoms or a cyclic thioalkoxy group containing 3 to 20 C atoms, a branched silyl group containing 3 to 20 C atoms or a cyclic silyl group containing 3 to 20 C atoms, a substituted keto group containing 1 to 20 C atoms, an alkoxycarbonyl group containing 2 to 20 C atoms, an aryloxycarbonyl group containing 7 to 20 C atom, a cyano group, a carbamoyl group, a haloformyl group, a formyl group, an isocyano group, an isocyanate group, a thiocyanate group, can isothiocyanate group, a hydroxyl group, a nitryl group, a CF₃ group, Cl, Br, F, a crosslinkable group, a substituted or non-substituted aromatic containing 5 to 40 ring atoms or a substituted or non-substituted heteroaromatic ring system containing 5 to 40 ring atoms, and an aryloxy containing 5 to 40 ring atoms or a heteroaryloxy group containing 5 to 40 ring atoms.

22. The organic compound according to claim 21, wherein Ar¹ contains at least one of D atom.

23. The organic compound according to claim 18, wherein the Ar¹ is one selected from the group consisting of formulas: 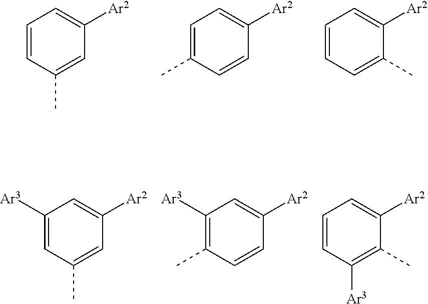
wherein Ar² and Ar³ are independently selected from an aryl group containing 5 to 60 ring atoms or a heteroaryl group containing 5 to 60 ring atoms.

24. The organic compound according to claim 23, wherein the Ar² and the Ar³ are independently selected from the group consisting of formulas: 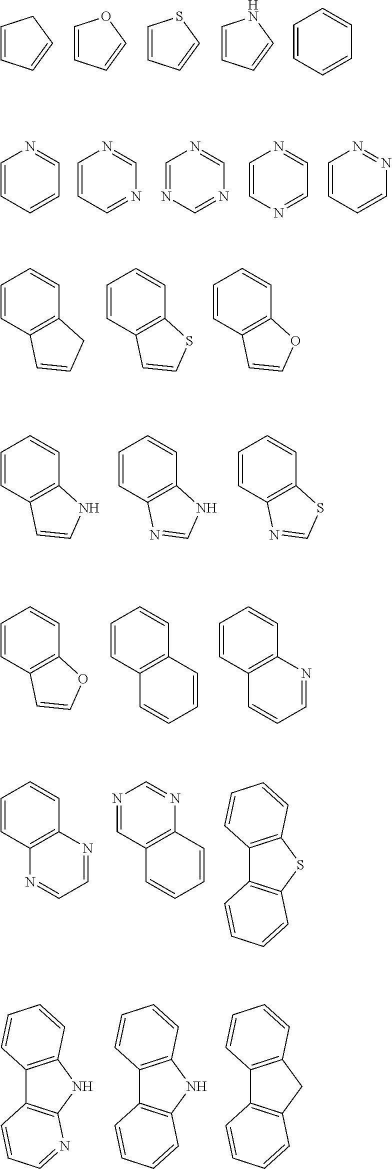 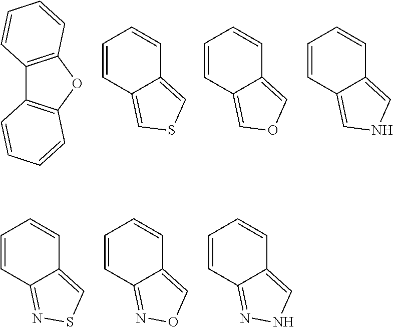
wherein H in the foregoing formulas may be optionally substituted.

25. The organic compound according to claim 18, wherein the Ar¹ is one selected from the group consisting of:

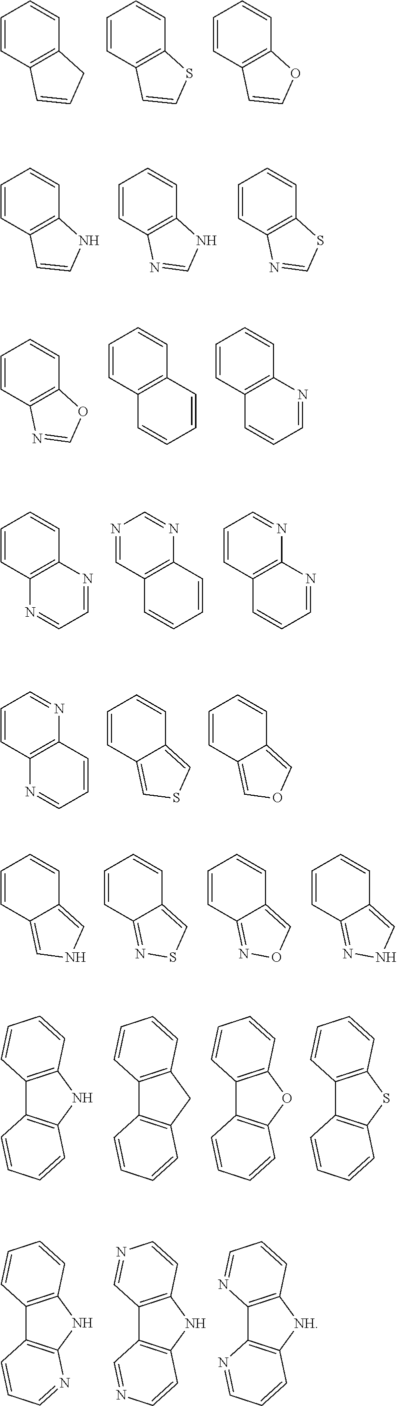

26. The organic compound according to claim 18, wherein the organic compound is selected from the structures represented by the following general formulas (2) to (8): 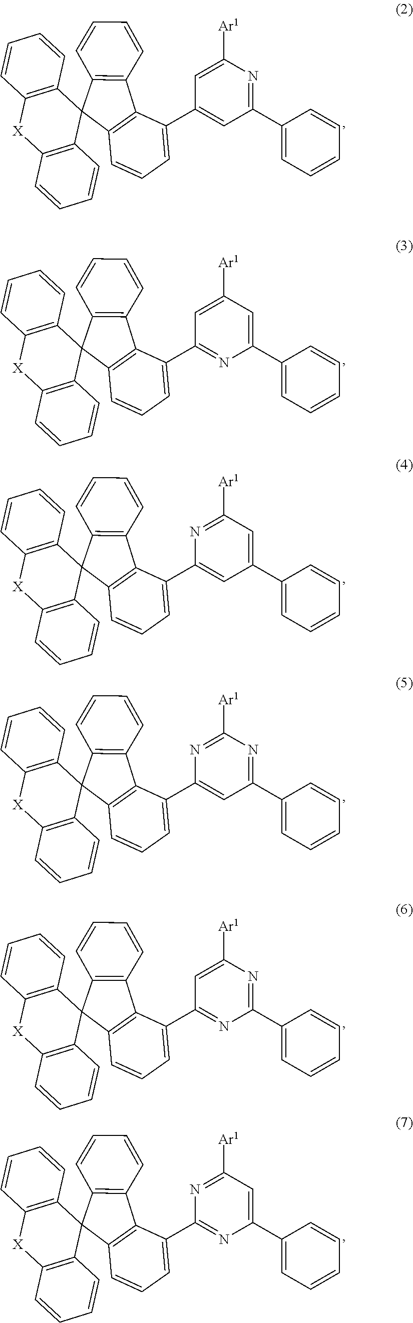 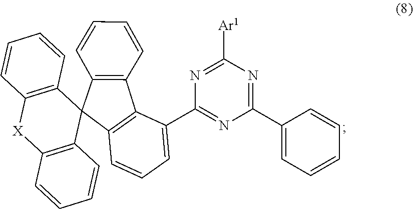
wherein X is independently selected from the group consisting of a single bond, N(R¹), C(R¹)₂, Si(R¹)₂, O, C═N(R¹), C═C(R¹)₂, P(R¹), P(═O)R¹, S, S═O, and SO₂; Ar¹ is selected from an aryl group containing more than 6 ring atoms and a heteroaryl group containing more than 6 ring atoms; R¹ is selected from the group consisting of H, D, F, CN, carbonyl, sulfonyl, alkoxy, an alkyl group containing 1 to 30 carbon atoms or a cycloalkyl group containing 3 to 30 carbon atoms, and an aryl group containing 5 to 60 ring atoms or a heteroaryl group containing 5 to 60 ring atoms.

27. The organic compound according to claim 18, wherein Ar¹ comprises an electron-donating group or an electron-accepting group.

28. The organic compound according to claim 27, wherein the electron-donating group is selected from the group consisting of:

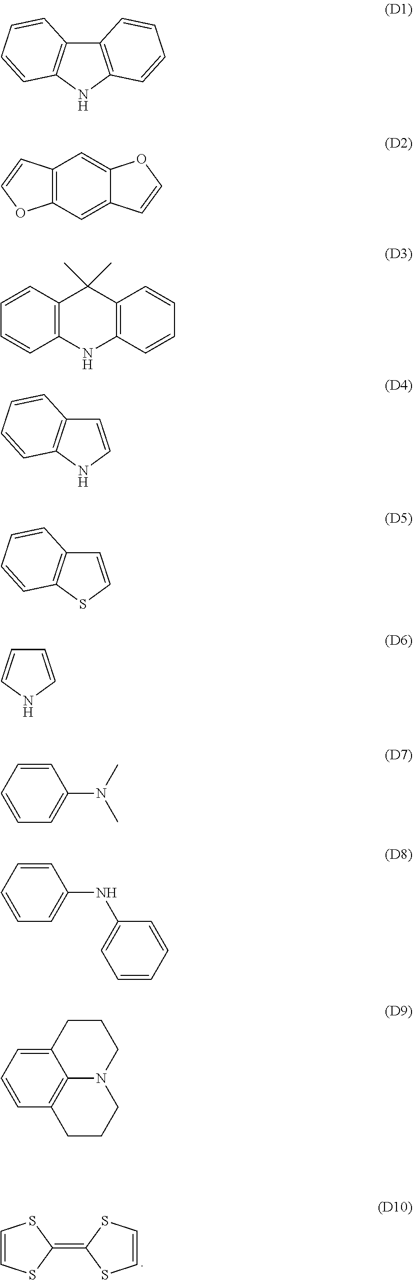

29. The organic compound according to claim 27, wherein the electron-accepting group is selected from the group consisting of F, a cyano group, and any one of the following groups; 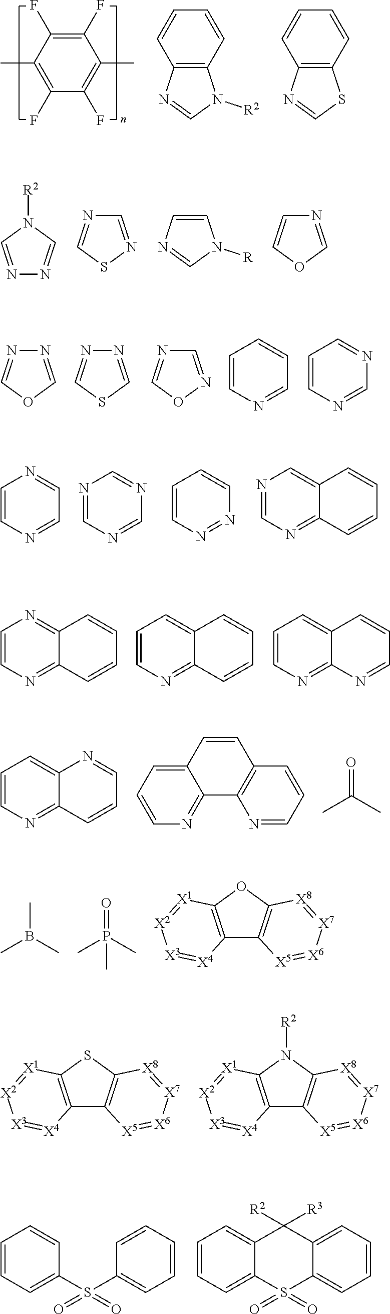 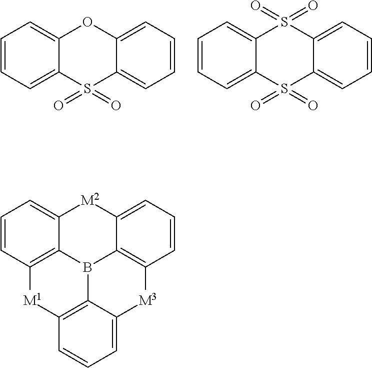
wherein n is selected from 1, 2 or 3; X¹ to X⁸ are independently selected from CR or N, and at least one of X¹ to X⁸ is selected from N; M¹, M² and/or M³ is not present, or M¹, M², and M³ are independently select from N(R), C(R)₂, Si(R)₂, O, C═N(R), C═C(R)₂, P(R), P(═O)R, S, S═O or SO₂; wherein R² and R³ are one or more independently selected from the group consisting of H, D, a linear alkyl group containing 1 to 20 C atoms, a linear alkoxy group containing 1 to 20 C atoms, a linear thioalkoxy group containing 1 to 20 C atoms, a branched alkyl group containing 3 to 20 C atoms or a cyclic alkyl group containing 3 to 20 C atoms, a branched alkoxy containing 3 to 20 C atoms or a cyclic alkoxy containing 3 to 20 C atoms, a branched thioalkoxy group containing 3 to 20 C atoms or a cyclic thioalkoxy group containing 3 to 20 C atoms, a branched silyl group containing 3 to 20 C atoms or a cyclic silyl group containing 3 to 20 C atoms, a substituted keto group containing 1 to 20 C atoms, an alkoxycarbonyl group containing 2 to 20 C atoms, an aryloxycarbonyl group containing 7 to 20 C atoms, a cyano group, a carbamoyl group, a haloformyl group, a formyl group, an isocyano group, an isocyanate group, a thiocyanate group, an isothiocyanate group, a hydroxyl group, a nitryl group, a CF₃ group, Cl, Br, F, a crosslinkable group, a substituted or non-substituted aromatic or heteroaromatic ring system containing 5 to 40 ring atoms and an aryloxy group containing 5 to 40 ring atoms or a heteroaryloxy group containing 5 to 40 ring atoms, wherein at least one of R² and R³ forms a monocyclic or polycyclic aliphatic or aromatic ring with a ring bonded to the group, or R² and R³ form a monocyclic or polycyclic aliphatic or aromatic ring with each other.

30. The organic compound according to claim 18, wherein the organic compound has a T₁≥2.2 eV; wherein the T₁ represents a first triplet excited state of the organic compound.

31. The organic compound according to claim 18, wherein the organic compound has (S1-T1) less than or equal to 0.20 eV; wherein the (S1-T1) represents an energy level difference between the first triplet excited state T₁ of the organic compound and the first singlet excited state S₁ of the organic compound.

32. The organic compound according to claim 18, wherein the organic compound is selected from the group consisting of:

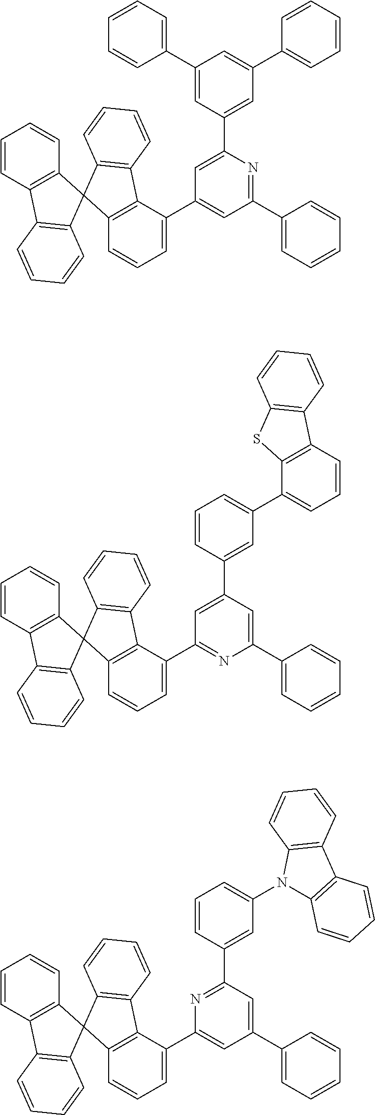 [SMILES: c1ccc(-c2cc(-c3ccccc3)cc(-c3cc(-c4cccc5c4-c4ccccc4C54c5ccccc5-c5ccccc54)cc(-c4ccccc4)n3)c2)cc1 ; c1ccc(-c2cc(-c3cccc(-c4cccc5c4sc4ccccc45)c3)cc(-c3cccc4c3-c3ccccc3C43c4ccccc4-c4ccccc43)n2)cc1 ; c1ccc(-c2cc(-c3cccc(-n4c5ccccc5c5ccccc54)c3)nc(-c3cccc4c3-c3ccccc3C43c4ccccc4-c4ccccc43)c2)cc1]

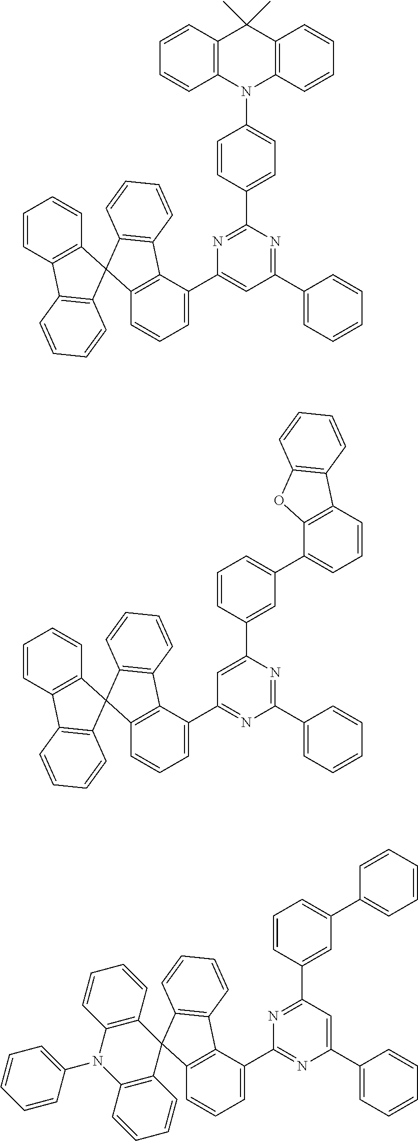 [SMILES: CC1(C)c2ccccc2N(c2ccc(-c3nc(-c4ccccc4)cc(-c4cccc5c4-c4ccccc4C54c5ccccc5-c5ccccc54)n3)cc2)c2ccccc21 ; c1ccc(-c2nc(-c3cccc(-c4cccc5c4oc4ccccc45)c3)cc(-c3cccc4c3-c3ccccc3C43c4ccccc4-c4ccccc43)n2)cc1 ; c1ccc(-c2cccc(-c3cc(-c4ccccc4)nc(-c4cccc5c4-c4ccccc4C54c5ccccc5N(c5ccccc5)c5ccccc54)n3)c2)cc1]

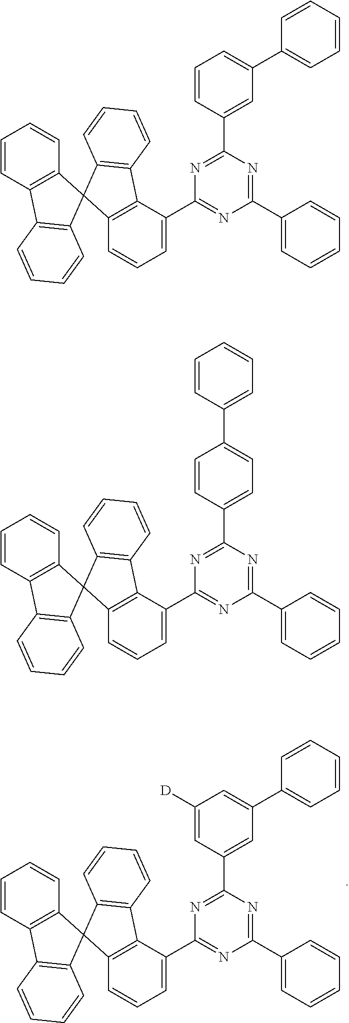 [SMILES: c1ccc(-c2cccc(-c3nc(-c4ccccc4)nc(-c4cccc5c4-c4ccccc4C54c5ccccc5-c5ccccc54)n3)c2)cc1 ; c1ccc(-c2ccc(-c3nc(-c4ccccc4)nc(-c4cccc5c4-c4ccccc4C54c5ccccc5-c5ccccc54)n3)cc2)cc1 ; [2H]c1cc(-c2ccccc2)cc(-c2nc(-c3ccccc3)nc(-c3cccc4c3-c3ccccc3C43c4ccccc4-c4ccccc43)n2)c1]

33. An organic mixture for an organic electronic device, comprising an organic compound according to claim 18 and an organic solvent or at least one organic functional material, wherein the organic functional material is selected from the group consisting of a hole injection material, a hole transporting material, a hole blocking material, an electron injection material, an electron transporting material, an electron blocking material, an organic host material, an organic dye, and a fluorescent material.

34. An organic electronic device, comprising a functional layer, wherein the functional layer comprises an organic compound according to claim 18.

35. The organic electronic device according to claim 34, wherein the organic electronic device is selected from the group consisting of an organic light-emitting diode, an organic photovoltaic cell, an organic light emitting cell, an organic field effect transistor, an organic light-emitting field effect transistor, an organic laser, an organic spintronic device, an organic sensor, and an organic plasmon emitting diode.

36. The organic electronic device according to claim 34, wherein the organic electronic device is an organic light-emitting diode comprising a light-emitting layer comprising the organic compound.

37. The organic electronic device according to claim 34, wherein the organic electronic device is an organic light-emitting diode comprising an electron transporting layer comprising the organic compound.
